(19) United States
(12) Patent Application Publication
(10) Pub. No.: US 2021/0032699 A1
(43) Pub. Date: Feb. 4, 2021

(54) MEASUREMENT OF GENOMIC AGE FOR PREDICTING THE RISK OF CANCER

(71) Applicant: University of Florida Research Foundation, Inc., Gainesville, FL (US)

(72) Inventor: Paul Gerson Okunieff, Gainesville, FL (US)

(21) Appl. No.: 16/091,977

(22) PCT Filed: Apr. 5, 2017
(86) PCT No.: PCT/US17/26061

§ 371 Date: Oct. 6, 2018

Related U.S. Application Data
(60) Provisional application No. 62/318,879, filed on Apr. 6, 2016.

(51) Int. Cl. C12Q 1/6886; C12Q 1/6858

(52) U.S. Cl. CPC C12Q 1/6886; C12Q 2600/156; C12Q 1/6858

(57) ABSTRACT

The subject invention pertains to materials and methods of determining accumulated mutations and the rate of mutations in a target genomic sequence which is a part of a short interspersed element (SINE). The assay utilizes a combination of a target sequence clamp and digital PCR (dPCR). The target sequence clamp prevents PCR amplification of the wild-type target sequence and permits PCR amplification of only the mutated target sequence. The dPCR provides the number of mutated target sequences per genome, which can be used to determine the rate of mutations in the target sequence, the accumulated mutations in the genome and/or the rate of mutations in the genome. The accumulated mutations and the rate of mutations in the target sequence and/or the genome can be used to determine the genomic age and/or the risk of cancer of a subject. A kit for performing the assay is also provided.

### CROSS-REFERENCE TO RELATED APPLICATIONS

This application claims the benefit of U.S. Provisional Application Ser. No. 62/318,879, filed Apr. 6, 2016, the disclosure of which is hereby incorporated by reference in its entirety, including all figures, tables and amino acid or nucleic acid sequences.

This application contains a sequence listing filed in electronic form as an ASCII.txt file entitled “T15956 (222110-1900) AS FILED Sequence Listing_ST25 2019_10_14” which was created on Oct. 11, 2019 and is 125 KB. The entire contents of the sequence listing are incorporated herein by reference in its entirety.

### BACKGROUND OF THE INVENTION

Due to a large number silent mutations and the absence of tools to detect them, the measurement of genome-wide chromosomal DNA abnormalities is not routine. The current next-generation sequencing (NGS) methods make inherent sequencing errors. The sequencing errors can be partially alleviated by increasing the number of runs and improving the purity of the sample; however, even deep sequencing methods suffer from false detection rates. Detection of genome-wide variance is rarely better than 1%. This is far from the 10⁶ sensitivity expected to be required for detecting the rate of silent mutations accumulating with each cell division or after an exposure to a mutagen, for example, a low-dose particle exposure. Therefore, the current NGS methods are insufficient to estimate genome-wide accumulated mutations and/or the rate of mutations, for example, point mutations and insertion/deletion (indel) mutations.

The premalignant genome-wide accumulated mutations and/or the rate of mutations usually cause few or no phenotypic effects but stochastically (randomly but extremely rarely) lead to driver mutations which cause cancer. The driver mutations accelerate the oncogenic mutational process and lead to evolutionary selection of more drivers in cancer-causing genes. The genome-wide accumulated mutations and/or the rate of mutations are dominated by point mutations (−95%) and short indel mutations (˜1-3%) and include a small component of translocations when high-linear energy transfer (LET) radiation is considered (˜1%). Driver mutations are far too complex to measure and interpret and are too infrequent to detect in small tissue volumes.

### BRIEF SUMMARY OF THE INVENTION

The invention relates to materials and methods of determining accumulated mutations and the rate of mutations in a target genomic sequence (also referred to herein as “a target sequence”), particularly a target sequence which is a part of a short interspersed element (SINE), a long interspersed element (LINE), any highly repeated sequence in a cell's genome, and/or the mitochondrial genome. Thus, the target sequence is present in a large number of copies per genome as SINEs, LINEs, or other highly repeated sequences within the genome of the cell, or the mitochondrial genome which is highly repeated in each cell in each mitochondrion. Because the entire mitochondrial genome is highly repeated due to the number of genome copies in each mitochondria and the number of mitochondria in each cell, the mitochondrial genome, in addition to a SINE and/or LINE, can serve as a target sequence in one embodiment of the invention. The assay to determine the number of accumulated mutations in a target sequence utilizes a combination of a target sequence clamp with digital PCR (dPCR). The target sequence clamp binds only to the wild-type target sequence, prevents PCR amplification of only the amplicons that have the wild-type target sequence and permits PCR amplification of only the amplicons that have the mutated target sequence. The dPCR, where the sample is separated into a large number of partitions, detects the presence of the DNA fragments containing the mutant target sequences in the large number of analyzed genomic DNA fragments. The PCR amplification in a partition of the sample indicates the presence of the mutant target sequence in that partition. The accumulated mutations in the target sequence can be calculated based on the number of fragments of the genomic DNA that arise from one genome and the number of fragments of the genomic DNA per genome that contain the mutated target sequence.

The accumulated mutations in a target sequence can be used to determine the rate of mutations in the target sequence, the accumulated mutations in the genome (genome-wide mutations) and the rate of mutations in the genome (genome-wide rate of mutations).

Accumulated mutations and the rate of mutations in the genome are directly proportional to genomic age and the risk of cancer. Accordingly, the invention also provides a method of calculating the genomic age and/or the risk of cancer in a subject. In one embodiment, the risk of cancer in a tissue or organ is determined.

Furthermore, the invention provides a kit to carry out the methods of the invention.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1. Natural Aging: baseline accumulation of mutations in a healthy astronaut with no risk factors. Nat+Direct Rad: incremental mutations due to the 3-year voyage. Nat+DirRMD+RRI: impact on the rates of accumulation of mutations due to radiation-induced mitochondrial damage (RMD) and radiation-related inflammation (RRI). Cancer distributions are based on reported levels of non-synonymous mutations in these types of tumors.

FIG. 2. Genomic age versus chronological age.

FIG. 3. Computing genomic age and rate of aging.

FIG. 4. Age is the dominant risk factor for cancer, outpacing smoking and PSA. Individuals under 20 years old have a 130-fold reduced risk of cancer compared to individuals over 75 years old.

FIG. 5. The passenger mutation (also known as silent or non-synonymous) rate increases with age, allowing for random cells to become cancerous. Different tissues can tolerate different numbers of mutations before cancers become common.

FIG. 6. QClamp™ utilizes a sequence-specific and wild-type template xenonucleotide “clamp” (XNA) that suppresses PCR amplification of only the wild-type template DNA and allows selective PCR amplification of only the mutant template DNA. This allows the detection of mutant DNA in the presence of a large excess of wild-type templates from any sample, including FFPE tissues and whole blood.

FIG. 7. dPCR results for QClamp™. Initial results demonstrating detection of 1 mutant target in 100,000 copies of wild-type targets is achievable with standard dPCR. Clamp2 was used for this study.

Forward Primer B1 F001
(SEQ ID NO: 1)
5′ CTTTAATCCCAGCACTCGGG-3′.
Reverse Primer B1 R001
(SEQ ID NO: 2)
5′-CTCTGTAGCCCTGGTGTCCTGG-3′.
Clamp1:
(SEQ ID NO: 4)
GGCTGGCCTCGAACTC; Tm = 79.4° C., 68.8% GC.
Clamp2:
(SEQ ID NO: 5)
GTGTCCTGGAACTCACTCTG; Tm = 78.9° C., 55% GC.

FIG. 8. B1 SINE Sequence Choice. The two XNA Clamps and the forward and reverse PCR primers were designed based on the highly conserved sequence shown as double underlined text (SEQ ID NO: 482).

FIG. 9. Example calculation of the fraction of B1 SINE sites with conserved CE3, LE2, or [LE2 or CE3] using an XNA multiplex. CE3 and LE2 are sequential (serially placed) in the PCR reading frame. DNA was collected from the livers of male NIH Swiss mice. PCR for B1 SINE was performed using primers alone, and the cycles required for detection in a 50-ng sample were set at 0.00. XNA CE3 was highly conserved among B1 loci at 79%, LE2 was also highly conserved at 86%, and [CE3 or LE2] were conserved on 97%. Alternatively to serial XNA placement, overlaying “allelic” XNA on the same locus can further improve sensitivity to detect mutations at that locus. Likewise, parallel clamps with independent PCR primers can be used to evaluate different chromosomes or DNA repair mechanisms.

FIG. 10. Fractions of total B1 SINE SITES with homology to the CE3 and LE2 regions. Potential to measure differential repair in areas with high and low baseline allelic conformity using serial XNA from the previous example. For example, B1 sequences that are highly conforming can provide a template on fidelity of repair. Serial XNA sequences, therefore, can be used to separately evaluate the 68% of the mouse genome that is very highly conforming.

FIG. 11. Impact of 1 Gy Total Body Irradiation on Liver B Mutation Levels Measured at 2 hr. DNA was collected from the livers of male NIH Swiss mice. PCR for B1 SINE was performed using primers alone, and the cycles required for detection in a 50-ng sample were set at 0.00. Mutations per base assumes 20 bases at risk for mutation for LE2, 16 bases for CE3, and a mouse genome size of 2.9 E9 bases. At 2 hours, DNA repair is known to only partially complete. Liver epithelium is known to repair damage with only a minority of cells undergoing apoptosis in the first 24 hr. Loci convergent with one or another majority loci repaired better than the loci that were divergent from the majority loci as evidenced by the reduced mutations in the CE3&LE2 group following radiation. This is presumed to be due to the availability of the majority template. This phenomenon allows for study of specific DNA repair mechanisms, in a personalized way.

FIG. 12. Impact of 9 Gy Total Body Irradiation on Spleen B1 Mutation Levels Measured at 2 hr. DNA was collected from the spleens of male NIH Swiss mice. PCR for B1 SINE was performed using primers alone, and the cycles required for detection in a 50-ng sample were set at 0.00. Mutations per base assumes 20 bases at risk for mutation for LE2, 16 bases for CE3, and a mouse genome size of 2.9 E9 bases. It is known that at 2 hr, little DNA repair occurs in lymphocytes; instead, they responded to 9 Gy at =4-6 hr with simultaneous apoptosis. Thus, the highly damaged lymphocytes were still present at 2 hr.

FIG. 13. Different organs from the same animal exhibited different frequencies and rates of mutation repair. DNA was collected from various organs of male NIH Swiss mice at 2 hr or 6 days after 1 or 9 Gy irradiation. Persistent mutation levels were intermediate in frequency and increased with dose for the liver. The mutations maintained for the 6-day endpoint for this slowly proliferative and low-apoptotic epithelial tissue. The spleen cleared mutations at day 6, presumably through lymphocyte apoptosis. The brain was resistant to mutations at any time point, and the small bowel, a rapidly proliferating tissue, had increasing mutation frequency with time as expected from silent mutations.

FIGS. 14A and 14B. Brain tissue of male NIH Swiss mice were resistant to mutations measured both early after exposure (2 h—FIG. 14A) and at later times (6 d—FIG. 14B). This was true for low doses (1 Gy) and high doses (9 Gy).

FIGS. 15A-15C. Brain tissue of male and female mice of radiation sensitive (BALB/c) mice (FIG. 15B) and radiation-resistant (C57BL/6) mice (FIG. 15A) were resistant to mutations in the brain measured at 24 hours. As seen with the serial XNA, the more resistant C57BL/6 strain appeared to use the majority allele to “repair” some mutated sequences (FIG. 15C). The resistance to mutations was seen in both sexes. Thus we can detect innate ability to repair DNA mutations.

FIG. 16. PCR primers were designed to flank highly conserved regions on the human LINE1. In each case, human genomic DNA was used (50 ng). Many produced fairly homogenous products and extremely high copy numbers, some detected in as little as 5-7 cycles. Primer set 6059 produced both homogeneous and plentiful product. Primer 279 also produced a homogeneous product. Both are excellent options for LINE regions for measuring genotoxicity. Examples for 6059 will be shown and primers for the detection of LINE1 are provided in FIG. 20.

FIG. 17. LINE1 abundance in 3 human gDNA sources (Ken, HFL1, HEK293) using primer set 6059. All had high abundance in a 50-ng gDNA sample, and all produced a reliable melting curve, indicating a homogeneous product.

FIG. 18. Shown are the 6059 LINE1 target sequence (SEQ ID NO: 483), the sections used to design PCR proprimerbes (in double underlining) and XNAs (single underlining) and the sequencing data from all 3 human DNA sources. The single underlined segment was 100% conserved, as determined by using deep gene sequencing and can be used to design XNA clamps. Deep gene sequencing is sensitive to about 1% per base. Several XNAs have can and have been developed from this lengthy and highly conserved sequence.

FIG. 19. Mitochondrial clamps. There are many suitable clamps, as the mitochondrial genome is well conserved and inherited almost exclusively from the mother. An example of a potential XNA from mouse Cyt A is shown. The XNA (SEQ ID NO: 484) and primer sequences (SEQ ID NOs: 485 and 486) are free from similar genomic sequences.

FIG. 20. PCR primers used for the amplification of LINE1.

FIGS. 21-23. Alignments of LINE1 sequences for the identification of clamp and primers identified in FIGS. 16 and 20. Continuous stars indicate conserved region suitable for clamp and primer design used in the experiments shown in FIG. 16 (sequence 6059; FIG. 21, sequence 279, FIG. 22, and sequence 139, FIG. 23).

FIG. 24. Line1 sequence for designing probes (double underlined) and clamps (single underlined sequence)(SEQ ID NO: 501). PCR primers are shown in bold.

### BRIEF DESCRIPTION OF THE SEQUENCES

SEQ ID NO: 1: Forward Primer B1.

SEQ ID NO: 2: Reverse Primer B1 R001.

SEQ ID NO: 3: Clamp1.

SEQ ID NO: 4: Clamp2.

SEQ ID NO: 5: Clamp3.

SEQ ID NO: 6: Sequence of mouse B1.

SEQ ID NOs: 7-10: Example of clamp sequences for Alu SINEs.

SEQ ID NOs: 11-98: Examples of target amplicons in Alu SINEs.

SEQ ID NOs: 99-274: Forward and reverse primers for clamp sequence of SEQ ID NOs: 7-10 and Alu SINE sequences of 11-98.

SEQ ID NOs: 275-316: Sequence of SINEs indicated in Table 1.

SEQ ID NOs: 317-416: Examples of frequent sequences in human genomes that can be used as clamp sequences.

SEQ ID NOs: 417-478: Examples of Alu sequences in humans.

SEQ ID NO: 479: An example of a forward primer for B1 having the sequence of SEQ ID NO: 281.

SEQ ID NO: 480: An example of a reverse primer for B1 having the sequence of SEQ ID NO: 281.

SEQ ID NO: 481: An example of a clamp for B1 having the sequence of SEQ ID NO: 281.

### DETAILED DISCLOSURE OF THE INVENTION

As used herein, the singular forms “a”, “an” and “the” are intended to include the plural forms as well, unless the context clearly indicates otherwise. Furthermore, to the extent that the terms “including”, “includes”, “having” “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” means within an acceptable error range for the particular value. In the context of compositions containing amounts of ingredients where the terms “about” or “approximately” are used, these compositions contain the stated amount of the ingredient with a variation (error range) of 0 to 10% around the value (X±10%).

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00, including the terminal values.

When ranges are used, such as for length of a SINE or a LINE or target sequence within a genome, primer or target sequence clamp, combinations and subcombinations of ranges (e.g., subranges within the disclosed ranges) and specific embodiments therein are intended to be explicitly included.

As used herein, the term “cancer” refers to the presence of cells possessing abnormal growth characteristics, such as uncontrolled proliferation, immortality, metastatic potential, rapid growth and proliferation rate, perturbed oncogenic signaling, and certain characteristic morphological features. This includes but is not limited to the growth of: (1) benign or malignant cells (e.g., tumor cells) that correlate with overexpression of a serine/threonine kinase; or (2) benign or malignant cells (e.g., tumor cells) that correlate with abnormally high levels of serine/threonine kinase activity or lipid kinase activity. Non-limiting serine/threonine kinases implicated in cancer include but are not limited to PI-3K mTOR and AKT. Exemplary lipid kinases include but are not limited to PI3 kinases such as PBKα, PBKβ, PBKδ, and PBKγ.

“Subject” refers to an animal, such as a mammal, for example a human. The methods described herein can be useful in both humans and non-human animals. In some embodiments, the subject is a mammal, and in some embodiments, the subject is human. The invention can be used in a subject selected from non-limiting examples of a human, non-human primate, rat, mouse, pig, dog or cat. Additional embodiments of the animals in which the invention can be practiced are well-known to a person of ordinary skill in the art and such embodiments are within the purview of the invention.

Where the term “and/or” is used within the application, it is intended that the elements recited within the phrase where the term “and/or” is used can be assess individually or in any combination of the recited elements. For example, the phrase “target SINE, target LINE and/or the genome” is meant to convey that each element of the phrase can be assessed individually or in any possible combination (e.g., target SINE alone, target LINE alone, genome alone, target SINE and target LINE in combination, target SINE and genome in combination, target LINE and genome in combination, or target SINE, target line, and genome in combination).

The invention relates to materials and methods of determining accumulated mutations and the rate of mutations in a target genomic sequence (also referred to herein as “a target sequence”), particularly a target sequence which is a part of a short interspersed element (SINE), a long interspersed element (LINE), any highly repeated sequence in a cell's genome, and/or the mitochondrial genome. Thus, the target sequence is present in a large number of copies per genome as SINEs, LINEs, or other highly repeated sequences within the genome of the cell. Thus, the invention provides an assay to determine the accumulated mutations and/or the rate of mutations in a target sequence within a short interspersed element (SINE), long interspersed elements (LINEs), mitochondrial genome and/or the genome (as used herein, the target sequence is any highly repeated sequence within the genome of a cell). The accumulated mutations and/or the rate of mutations in a target SINE, target LINE and/or the genome integrate various causes of DNA damage. As used herein, the term “genome” refers to highly repeated sequences within the genome of a cell, such as mitochondrial genomes. “Highly repeated sequences” are a nucleotide sequence that is repeated hundreds to thousands of times within the genome of a cell. As such, the invention provides materials and methods for quantitative estimation of mutations and rate of mutations. The invention also provides an assay to measure point mutations and indels (which together comprise >95% of all mutations) in a target sequence within a cell a target SINE and/or target LINE for example, Alu in humans or B1 in mice or a target LINE, such as those provided in Tables 6-7 (which provides GenBank Accession numbers for partial LINE1 sequences and full length LINE1 sequences for humans and mice, each of which is hereby incorporated by reference in their entirety). The accumulated mutations and/or the rate of mutations in a target sequence within a target SINE or genome of the cell can be extrapolated to measure point mutations and indels in the genome.

The current approaches to estimating cancer risk require screening many animals for long periods of time. These methods generally feature genetically defined animals with driver mutations that cause cancers and incidence rates that are not representative of spontaneously occurring human cancers. The invention provides cancer risk assessments that can be performed on a subject-by-subject basis and on an organ-by-organ basis, thus allowing for subject-specific and organ-specific estimates of cancer risk. The methods of the invention can also be used to determine the effects on cancer risk of genetic and non-genetic factors, for example, race, family lineage, and environmental factors such as food, lifestyle choices, smoking, etc.

The accumulated mutations and/or the rate of mutations in a target sequence within the target SINE, target LINE, and/or the genome is directly proportional to the individual's chronological age. Specifically, an individual with a higher chronological age has more accumulated mutations and/or a higher rate of mutations in a target sequence within a target SINE, target LINE, and/or the genome compared to an individual with a relatively lower chronological age. An embodiment of the invention provides an assay to determine accumulated mutations and/or the rate of mutations in a target sequence in a target SINE, target LINE and/or the genome. The genome-wide accumulated mutations and the rate of mutations can be used to estimate genomic age of an individual for correlation with the individual's chronological age.

For the purpose of the invention, the phrase “chronological age” is the age of a subject based on the subject's date of birth. Accordingly, compared to a chronologically younger subject, a chronologically older subject has a date of birth earlier in time.

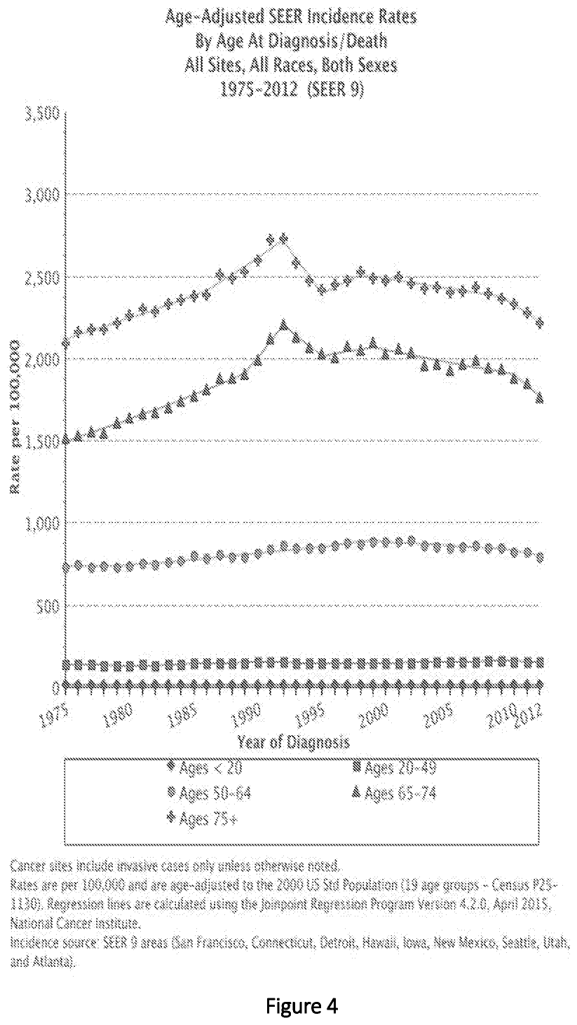

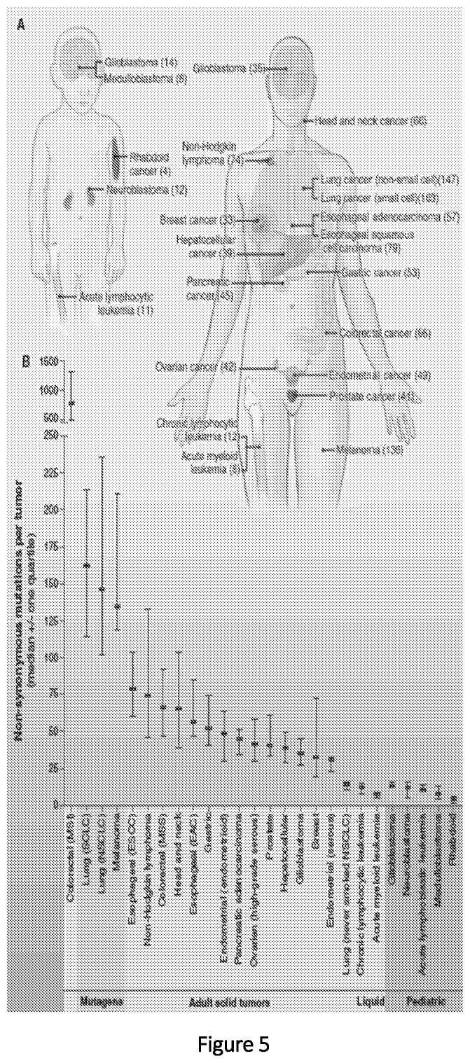

Age-related DNA damage is random, is not confined to coding regions and increases with age. Therefore, the number of mutations in certain tumors is directly proportional to the age of the patient (FIG. 4). Ninety-five percent (95%) of these premalignant mutations (passengers) are point mutations (e.g., C>G), and many of the remaining mutations are short indels (e.g., CTT>CT or CT>CTT). The inter-individual rate of mutations, which indicates different genomic age between individuals, is determined by a host of hereditary, acquired, and environmental factors, including radiation exposure.

While almost all mutations are “passenger” mutations, i.e., silent mutations, occasionally a “driver” mutation occurs. Driver mutations confer a competitive advantage upon the reproduction of the affected cell. Thus, while it would be difficult to detect cells with a driver mutation without complete organs to study and without unrealistic databases and sequencing resources, passenger mutation frequencies can be used to mathematically determine driver mutation frequency and downstream cancer incidence. The process from driver mutation to tumor is typically estimated to be 1 to 15 years, and tumors typically have only a few driver mutations (≥10). Notably, 10 times fewer driver mutations are affected by chromosome changes than by point mutations, and high-LET radiation induces both types of mutations.

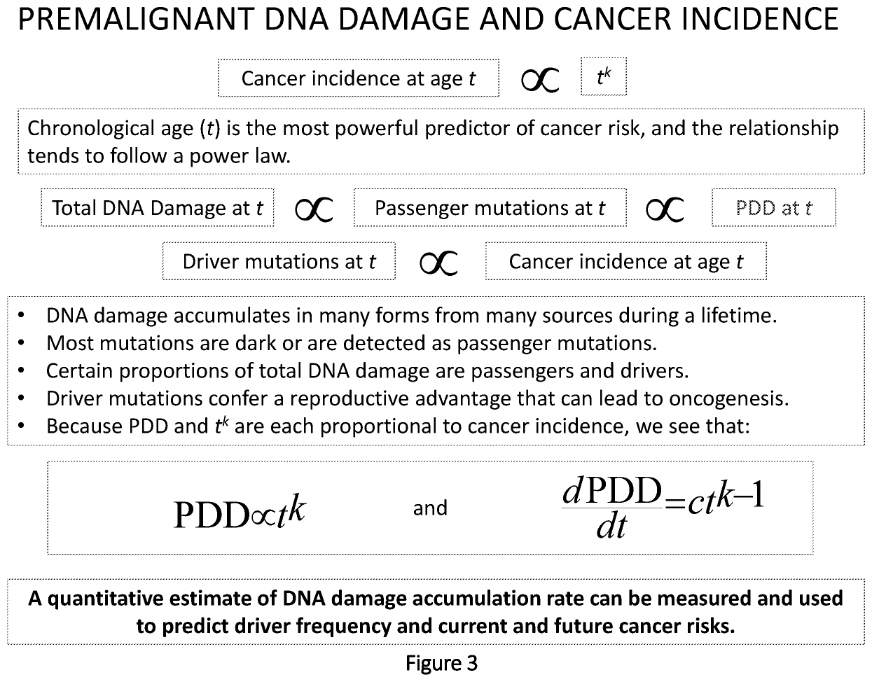

Since the rate of mutations, and consequently, the chances of the occurrence of driver mutations increase with increasing chronological age, chronological age typically correlates with cancer risk in the general population, i.e., higher chronological age of a subject typically, but not necessarily, indicates higher risk of cancer in the subject. For example, cancer incidence per 100,000 is 17 for ages less than 20, which increases by a factor of 10 to 157 for ages 20-49, increases another 5-fold for ages 50 to 64, and further increases another 3-fold for ages over 75 (>2,200/100,000), for a total increase of more than 130-fold (FIG. 3). These fold-changes indicate that increasing age impacts death and incidence of cancer more than smoking.

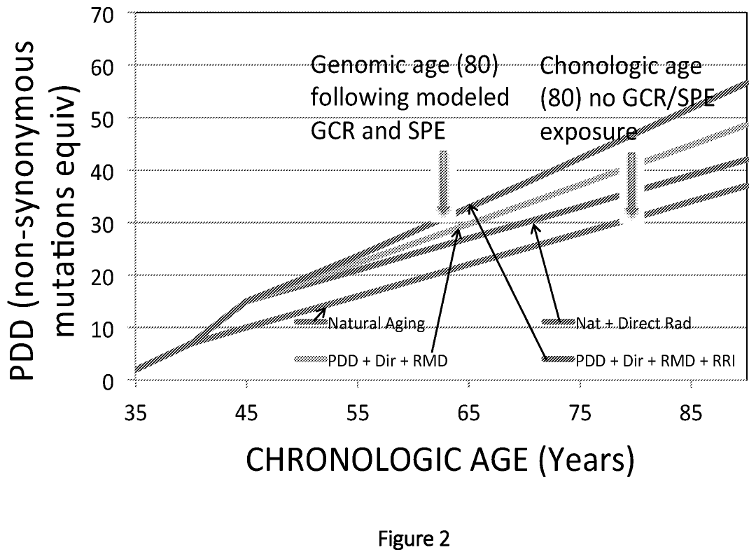

For the purpose of the invention, the “genomic age” indicates the accumulated mutations and/or the rate of mutations in the genome of a subject (FIG. 2) as they relate to the average accumulated mutations and/or the average rate of mutations in the genome of a subject of a particular chronological age. For example, if the accumulated mutations and/or the rate of mutations in the genome of a subject 30 years of age is equal to the average accumulated mutations and/or the average rate of mutations in the genome of a subject 40 years of age, then the genomic age of the subject 30 years of age is 40 years. Therefore, a genome having more accumulated mutations and/or a higher rate of mutations is an “older genome” compared to a “younger genome” having relatively fewer accumulated mutations and/or a lower rate of mutations.

The comparison of a subject's chronological age and genomic age can be expressed as genomic age−the chronological age (Δ_{age}). Therefore, a positive Δ_{age} indicates that a subject is aging at a higher rate than average, whereas a negative Δ_{age} indicates that a subject is aging at a lower rate than average

A standard scale for the genomic age for a particular species can be determined based on the average accumulated mutations and/or the average rate of mutations in different groups of individuals of varying ages. As such, the standard scale for the genomic age for the species indicates the average accumulated mutations and/or the average rate of mutations in a target sequence in a target SINE, target LINE, and/or the genomes of individuals belonging to the species at increasing chronological ages.

Accordingly, the invention provides methods for measuring accumulated mutations and/or the rate of mutations in a target sequence in a target SINE, target LINE and/or the genome to determine the genomic age of a subject and, consequently, to determine the cancer risk of the subject. Genomic age and chronological age of a subject can be compared to the known chronological age of the subject and the standard scale of genomic age to identify the subject's risk for cancer and offer enhanced cancer screening if the subject has a higher risk of cancer. Low-risk groups, on the other hand, can be spared from unnecessary screening tests. The accumulated mutations and/or mutation rates can also be used to evaluate the impact of environment (e.g., insecticides), lifestyle changes (e.g., weight loss or smoking cessation), and therapies (e.g., X-rays, medications) on genotoxic load, mutation rate, and, consequently, cancer risk.

Accordingly, an embodiment of the invention provides an assay to determine the number of accumulated mutations in a target sequence within a target SINE and/or target LINE and/or genome of a subject. For the purpose of this invention, this assay is called the clamp/dPCR combination assay.

The clamp/dPCR combination assay comprises the steps of:

a) obtaining a genomic DNA sample from the subject and fragmenting the genomic DNA sample, or obtaining a fragmented genomic DNA sample from the subject,

b) mixing a predetermined number of fragments of the genomic DNA that arise from a predetermined number of genomes with a reagent mixture to produce a reaction mixture, the reagent mixture comprising:

i) a pair of polymerase chain reaction primers that amplify a target amplicon comprising the target sequence within the target SINE,

ii) a target sequence clamp which binds only to the wild-type target sequence within the SINE, wherein the target sequence clamp prevents the PCR amplification of only those target amplicons that have the target wild-type sequence within the SINE and permits the PCR amplification of only those target amplicons that have the target mutated sequence within the SINE, and

iii) a DNA polymerase enzyme and the reactants for a digital PCR (dPCR),

c) subjecting the reaction mixture to the dPCR,

d) identifying the number of fragments of the genomic DNA comprising the target amplicon having the target mutated sequence within the SINE based on the number of positive PCR amplifications in the dPCR,

e) calculating the number of accumulated mutations per genome in the target sequence within the target SINE and/or target LINE and/or genome based on the number of fragments of the genomic DNA that arise from one genome and the number of fragments of the genomic DNA per genome that comprise the target amplicons having the target mutated sequence within the target SINE and/or target LINE and/or genome wherein the presence of the target mutated sequence within the target SINE and/or target LINE and/or genome is indicated by the positive PCR amplification in the dPCR.

LINEs are transposons that are about 5-6 kb long, contain an internal polymerase II promoter and encode two open reading frames (ORFs). Upon translation, a LINE RNA assembles and moves to the nucleus, where an endonuclease activity makes a single-stranded nick and the reverse transcriptase uses the nicked DNA to prime reverse transcription from the 3′ end of the LINE RNA. Reverse transcription frequently fails to proceed to the 5′ end, resulting in many truncated, nonfunctional insertions. Most LINE-derived repeats are short, with an average size of 900 bp for all LINE1 copies, and a median size of 1,070 bp for copies of the currently active LINE1 element. Three distantly related LINE families are found in the human genome: LINE1, LINE2 and LINE3, with LINE1 being the only remaining active LINE. Exemplary target LINE1 sequences are provided in Tables 6-7, which provide both partial and full length LINE1 sequences for humans and mice, identified by GenBank accession number. Other LINE1 sequences, including those of other animal species, are known in the art and can be easily identified in various databases, such as GenBank.

A SINE is a highly repetitive sequence that retrotransposes into a eukaryotic genome through intermediates transcribed by RNA polymerase III (pol III). In many species, SINEs are ubiquitously dispersed throughout the genome and can constitute a significant mass fraction of total genome, for example, typically about 10% or even above 10% in some cases.

SINEs cause mutations both by their retrotransposition within genes and by unequal recombination.

SINEs are relatively short (<700 bp) nonautonomous retroposons transcribed by pol III from an internal promoter and reverse transcribed by the reverse transcriptase of long interspersed elements. Eukaryotic genomes typically contain hundreds of thousands, and sometimes even more, of SINE copies (see Table 1, column: copy number). A SINE typically consists of a head, body and tail. The 5′-terminal head originates from one of the cellular RNAs synthesized by pol III: tRNA, 7SL RNA or SS rRNA; the body can contain a central domain which may be shared by distant SINE families; and the 3′-terminal tail is a sequence of variable length consisting of simple and often degenerate repeats. Various aspects of SINE structure, biology and evolution have been reviewed in Vassetzky et al. (2012). Vassetzky et al. also provide a database of SINE families from animals, flowering plants and green algae (see sines.eimb.ru). Non-limiting examples of SINEs from certain animals from the database provided by Vassetzky et al. are provided in Table 1. Several variants of the SINEs provided in Table 1 are also described by Vassetzky et al. (see sines.eimb.ru). Additional examples of SINEs suitable according to the methods of the claimed invention are well-known to a person of ordinary skill in the art and such embodiments are within the purview of the invention.

TABLE 1
Non-limiting examples of SINEs from certain animals.

| SINE | | | SEQ |
|---|---|---|---|
| name | Organism | Copy number | ID NO: |
| AFC | African cichlids | 2 × 10³-2 × 10⁴ | 275 |
| AFC-2 | African cichlids | 2.4 × 10³ | 276 |
| AFC-3 | African cichlids | 8 × 10² | 277 |
| AfroLA | elephants and | 7 × 10⁵ | 278 |
| | mammoths | (mammoth) | |
| | (Proboscidea) | | |
| AfroSINE | afrotherians | 8 × 10⁵ | 279 |
| Alu | primates | 1.1 × 10⁶ | 280 |
| | | (human) | |
| | | 1.5 × 10⁵ | |
| | | (galago) | |
| B1 | Mus musculus | 8 × 10³-6.5 × 10⁵ | 281 |
| B1-dID | Gliridae, Sciuridae & | 1.1 × 10⁵ | 282 |
| | Aplodontidae | (dormouse) | |
| | | 4.5 × 10⁴ | |
| | | (squirrel) | |
| | | 2.8 × 10⁴ | |
| | | (beaver) | |
| B4 | different rodent | 4 × 10⁵ | 283 |
| | families | (mouse) | |
| | | 3.6 × 10⁵ | |
| | | (rat) | |
| Bovc-tA2 | Bovidae | 2 × 10⁴ | 284 |
| | (cattle, goats & | | |
| | sheep) | | |
| Bov-tA | Bovidae | 2 × 10⁵ | 285 |
| | (cattle, goats & | | |
| | sheep) | | |
| C | Lagomorpha | 5.4 × 10⁵ | 286 |
| CAN | Carnivora | 2 × 10⁵ | 287 |
| CHRS-1 | cetaceans, | 10⁴-10⁵ | 288 |
| | hippopotamuses, | | |
| | ruminants & | | |
| | suiforms | | |
| CHRS-2 | cetaceans, | 10⁴-10⁵ | 289 |
| | hippopotamuses, | | |
| | ruminants & | | |
| | suiforms | | |
| EC-1 | horse | 7.7 × 10³ | 290 |
| | (Equus caballus) | | |
| ERE-A | horses | 9 × 10⁴ | 291 |
| | (Equus spp.) | | |
| ERE-B1 | horse | 3.8 × 10⁵ | 292 |
| | (Equus caballus) | | |
| ERE-B2 | horse | 3.8 × 10⁵ | 293 |
| | (Equus caballus) | | |
| Fc-1 | Carnivora | 4 × 10⁴-6 × 10⁴ | 294 |
| | | (cat, dog, panda) | |
| ID | rodents | 2.5 × 10⁴-5 × 10⁵ | 295 |
| LF | coelacanths & | 10⁵ | 296 |
| | tetrapods | (Latimeria | |
| | (Sarcopterygii) | menadoensis) | |
| | | 3 × 10² | |
| | | (mammals) | |
| Mac1 | kangaroos | 4.5 × 10⁴ | 297 |
| | (Macropodoidea) | (wallaby) | |
| MamSINE1 | mammals | 5 × 10² | 298 |
| | | (platypus) | |
| Mar1 | marsupials | 5 × 10⁵ | 299 |
| | (Metatheria) | (opossum) | |
| Mar3 | marsupials | 5.3 × 10⁴ | 300 |
| | (Metatheria) | (wallaby) | |
| Mare3 | mammals | 1 × 10² | 301 |
| | | (human) | |
| | | 1.4 × 10³ | |
| | | (opossum) | |
| MEN | squirrels (Menetes & | 1 × 10⁵ | 302 |
| | Callosciurus) | | |
| Mon-1 | monotremes | 2 × 10⁶ | 303 |
| | | (platypus) | |
| Pca-1 | hyrax | 9.3 × 10³ | 304 |
| | (Procavia capensis) | | |
| Ped-1 | springhare | 6 × 10⁴ | 305 |
| | (Pedetes capensis) | | |
| Ped-2 | springhare | 2.5 × 10⁵ | 306 |
| | (Pedetes capensis) | | |
| PinS-1 | spruces | 2.7 × 10² | 307 |
| | (Picea spp.) | | |
| PRE-1 | pigs and peccaries | 10⁶ | 308 |
| | | (Pig) | |
| Rhin-1 | bat families | | 309 |
| | Rhinolophidae & | | |
| | Hipposideridae | | |
| RSINE-1 | mouse & rat | 3 × 10⁴ | 310 |
| | | (mouse) | |
| | | 1.6 × 10⁴ | |
| | | (rat) | |
| SS-1 | Pig | 1.9 × 10⁵ | 311 |
| | (Sus scrofa) | | |
| STRIDM | thirteen-lined | 5.4 × 10³ | 312 |
| | ground squirrel | | |
| | (Spermophilus | | |
| | tridecemlineatus) | | |
| TAL | moles | 1 × 10⁵ | 313 |
| | (Talpidae) | | |
| Ther-1 | mammals, birds & | 4 × 10⁵ | 314 |
| | reptiles | (human) | |
| | | 1 × 10⁵ | |
| | | (mouse, rat) | |
| Ther-2 | marsupials & | 8 × 10⁴ | 315 |
| | placentals | (human) | |
| WallSI4 | wallaby | 1 × 10⁵ | 316 |
| | (Macropus eugenii) | | |

A SINE and/or LINE and/or target sequence within the genome used in an embodiment of the invention is spread throughout the genome and covers at least a certain percentage of the entire genome, and thus forms a representative sample of the entire genome. For example, a preferred SINE and/or LINE and/or target sequence within the genome for use in the assay of the invention typically covers about 4-15%, about 5-14%, about 6-13%, about 7-12%, about 8-11%, about 9-10% or about 10% of the genome.

The SINE and/or LINE and/or target sequence within the genome used for a particular assay depends on the species to which the subject belongs and the prevalence of the SINE and/or LINE and/or target sequence within the genome in the species' genome. A preferred SINE and/or LINE and/or target sequence within the genome for use in the assay of the invention typically comprises about 50-500, about 100-400, about 100-250, about 200-300, about 250-350 or about 300 bp.

A person of ordinary skill in the art can identify a suitable SINE for use in a particular species. Also, depending on the sequence of the SINE and/or LINE and/or target sequence within the genome, a person of ordinary skill in the art can design a target sequence clamp and a primer pair to conduct the assay of the invention. Such embodiments are within the purview of the invention.

The target sequence and, accordingly, the clamp can be designed based on the sequence of the SINE and/or LINE and/or target sequence within the genome. A person of ordinary skill in the art can appreciate that variation exists even within the large number of copies of a particular SINE and/or LINE and/or target sequence within the genome throughout the genome. However, within the larger sequence of a SINE and/or LINE and/or target sequence within the genome, certain portions of the SINE and/or LINE and/or target sequence within the genome do not show much variability among the copies of the SINE and/or LINE and/or target sequence within the genome throughout the genome. For example, in humans, Alu is about 300 bp long; however, portions of Alu that are about 5-50 bp, about 10-40 bp, about 20-30 bp, about 25 bp or about 10-15 bp are highly conserved among the large number of copies of Alu throughout the genome. In certain embodiments, the clamp sequences are designed based on sequences that are conserved across different human races or animal breeds/strains.

In one embodiment, the clamp sequence is selected from the sequences provided in Table 2 below. These sequences are the most common sequences that occur in human Chromosome 1.

TABLE 2
Examples of frequent sequences in
Homo sapiens genome, Chromosome 1.

| SEQ ID No. | Frequency | Percentage | SEQ ID NO. |
|---|---|---|---|
| 1 | 120976 | 0.0536 | 317 |
| 2 | 120236 | 0.0532 | 318 |
| 3 | 30120 | 0.0133 | 319 |
| 4 | 30056 | 0.0133 | 320 |
| 5 | 29754 | 0.0132 | 321 |
| 6 | 29720 | 0.0132 | 322 |
| 7 | 28146 | 0.0125 | 323 |
| 8 | 27970 | 0.0124 | 324 |
| 9 | 21179 | 0.0094 | 325 |
| 10 | 20808 | 0.0092 | 326 |
| 11 | 20167 | 0.0089 | 327 |
| 12 | 20161 | 0.0089 | 328 |
| 13 | 19956 | 0.0088 | 329 |
| 14 | 19794 | 0.0088 | 330 |
| 15 | 19704 | 0.0087 | 331 |
| 16 | 19690 | 0.0087 | 332 |
| 17 | 19685 | 0.0087 | 333 |
| 18 | 19590 | 0.0087 | 334 |
| 19 | 19389 | 0.0086 | 335 |
| 20 | 19336 | 0.0086 | 336 |
| 21 | 19243 | 0.0085 | 337 |
| 22 | 19076 | 0.0084 | 338 |
| 23 | 19061 | 0.0084 | 339 |
| 24 | 19035 | 0.0084 | 340 |
| 25 | 19002 | 0.0084 | 341 |
| 26 | 18958 | 0.0084 | 342 |
| 27 | 18926 | 0.0084 | 343 |
| 28 | 18851 | 0.0083 | 344 |
| 29 | 18837 | 0.0083 | 345 |
| 30 | 18734 | 0.0083 | 346 |
| 31 | 18624 | 0.0082 | 347 |
| 32 | 18563 | 0.0082 | 348 |
| 33 | 18526 | 0.0082 | 349 |
| 34 | 18498 | 0.0082 | 350 |
| 35 | 18397 | 0.0081 | 351 |
| 36 | 18390 | 0.0081 | 352 |
| 37 | 18385 | 0.0081 | 353 |
| 38 | 18335 | 0.0081 | 354 |
| 39 | 18312 | 0.0081 | 355 |
| 40 | 18231 | 0.0081 | 356 |
| 41 | 18150 | 0.008 | 357 |
| 42 | 18046 | 0.008 | 358 |
| 43 | 17676 | 0.0078 | 359 |
| 44 | 17616 | 0.0078 | 360 |
| 45 | 17498 | 0.0077 | 361 |
| 46 | 17486 | 0.0077 | 362 |
| 47 | 16988 | 0.0075 | 363 |
| 48 | 16945 | 0.0075 | 364 |
| 49 | 16913 | 0.0075 | 365 |
| 50 | 16856 | 0.0075 | 366 |
| 51 | 16797 | 0.0074 | 367 |
| 52 | 16758 | 0.0074 | 368 |
| 53 | 16518 | 0.0073 | 369 |
| 54 | 16483 | 0.0073 | 370 |
| 55 | 16452 | 0.0073 | 371 |
| 56 | 16409 | 0.0073 | 372 |
| 57 | 16387 | 0.0073 | 373 |
| 58 | 16380 | 0.0073 | 374 |
| 59 | 16378 | 0.0073 | 375 |
| 60 | 16342 | 0.0072 | 376 |
| 61 | 16278 | 0.0072 | 377 |
| 62 | 16245 | 0.0072 | 378 |
| 63 | 16011 | 0.0071 | 379 |
| 64 | 15994 | 0.0071 | 380 |
| 65 | 15978 | 0.0071 | 381 |
| 66 | 15974 | 0.0071 | 382 |
| 67 | 15942 | 0.0071 | 383 |
| 68 | 15941 | 0.0071 | 384 |
| 69 | 15920 | 0.007 | 385 |
| 70 | 15882 | 0.007 | 386 |
| 71 | 15854 | 0.007 | 387 |
| 72 | 15838 | 0.007 | 388 |
| 73 | 15820 | 0.007 | 389 |
| 74 | 15798 | 0.007 | 390 |
| 75 | 15797 | 0.007 | 391 |
| 76 | 15770 | 0.007 | 392 |
| 77 | 15725 | 0.007 | 393 |
| 78 | 15687 | 0.0069 | 394 |
| 79 | 15658 | 0.0069 | 395 |
| 80 | 15650 | 0.0069 | 396 |
| 81 | 15490 | 0.0069 | 397 |
| 82 | 15444 | 0.0068 | 398 |
| 83 | 14881 | 0.0066 | 399 |
| 84 | 14799 | 0.0066 | 400 |
| 85 | 14407 | 0.0064 | 401 |
| 86 | 14283 | 0.0063 | 402 |
| 87 | 14227 | 0.0063 | 403 |
| 88 | 14176 | 0.0063 | 404 |
| 89 | 13398 | 0.0059 | 405 |
| 90 | 13334 | 0.0059 | 406 |
| 91 | 13320 | 0.0059 | 407 |
| 92 | 13309 | 0.0059 | 408 |
| 93 | 13303 | 0.0059 | 409 |
| 94 | 13283 | 0.0059 | 410 |
| 95 | 13273 | 0.0059 | 411 |
| 96 | 13135 | 0.0058 | 412 |
| 97 | 12905 | 0.0057 | 413 |
| 98 | 12792 | 0.0057 | 414 |
| 99 | 12748 | 0.0056 | 415 |
| 100 | 12682 | 0.0056 | 416 |

Therefore, in one embodiment, the target sequence, and, accordingly, the clamp, is designed based on the highly conserved portion of a particular SINE.

Examples of Alu SINES, suitable clamp sequences for particular Alu SINES and corresponding primer pairs are given in Table 3.

TABLE 3
Examples of combinations of clamp sequences,
target amplicon SINEs and primer pairs.

| | Target amplicon | | |
|---|---|---|---|
| Clamp Sequence | in Alu SINE | Primer 1 | Primer 2 |
| (SEQ ID NO:) | (SEQ ID NO:) | (SEQ ID NO:) | (SEQ ID NO:) |
| 7 | 11 | 99 | 100 |
| 7 | 12 | 101 | 102 |
| 7 | 13 | 103 | 104 |
| 7 | 14 | 105 | 106 |
| 7 | 15 | 107 | 108 |
| 7 | 16 | 109 | 110 |
| 7 | 17 | 111 | 112 |
| 7 | 18 | 113 | 114 |
| 7 | 19 | 115 | 116 |
| 7 | 20 | 117 | 118 |
| 7 | 21 | 119 | 120 |
| 7 | 22 | 121 | 122 |
| 7 | 23 | 123 | 124 |
| 7 | 24 | 125 | 126 |
| 7 | 25 | 127 | 128 |
| 7 | 26 | 129 | 130 |
| 7 | 27 | 131 | 132 |
| 7 | 28 | 133 | 134 |
| 7 | 29 | 135 | 136 |
| 7 | 30 | 137 | 138 |
| 7 | 31 | 139 | 140 |
| 7 | 32 | 141 | 142 |
| 8 | 33 | 143 | 144 |
| 8 | 34 | 145 | 146 |
| 8 | 35 | 147 | 148 |
| 8 | 36 | 149 | 150 |
| 8 | 37 | 151 | 152 |
| 8 | 38 | 153 | 154 |
| 8 | 39 | 155 | 156 |
| 8 | 40 | 157 | 158 |
| 8 | 41 | 159 | 160 |
| 8 | 42 | 161 | 162 |
| 8 | 43 | 163 | 164 |
| 8 | 44 | 165 | 166 |
| 8 | 45 | 167 | 168 |
| 8 | 46 | 169 | 170 |
| 8 | 47 | 171 | 172 |
| 8 | 48 | 173 | 174 |
| 8 | 49 | 175 | 176 |
| 8 | 50 | 177 | 178 |
| 8 | 51 | 179 | 180 |
| 8 | 52 | 181 | 182 |
| 8 | 53 | 183 | 184 |
| 9 | 54 | 185 | 186 |
| 9 | 55 | 187 | 188 |
| 9 | 56 | 189 | 190 |
| 9 | 57 | 191 | 192 |
| 9 | 58 | 193 | 194 |
| 9 | 59 | 195 | 196 |
| 9 | 60 | 197 | 198 |
| 9 | 61 | 199 | 200 |
| 9 | 62 | 201 | 202 |
| 9 | 63 | 203 | 204 |
| 9 | 64 | 205 | 206 |
| 9 | 65 | 207 | 208 |
| 9 | 66 | 209 | 210 |
| 9 | 67 | 211 | 212 |
| 9 | 68 | 213 | 214 |
| 9 | 69 | 215 | 216 |
| 9 | 70 | 217 | 218 |
| 9 | 71 | 219 | 220 |
| 9 | 72 | 221 | 222 |
| 9 | 73 | 223 | 224 |
| 9 | 74 | 225 | 226 |
| 9 | 75 | 227 | 228 |
| 9 | 76 | 229 | 230 |
| 9 | 77 | 231 | 232 |
| 9 | 78 | 233 | 234 |
| 9 | 79 | 235 | 236 |
| 9 | 80 | 237 | 238 |
| 9 | 81 | 239 | 240 |
| 9 | 82 | 241 | 242 |
| 9 | 83 | 243 | 244 |
| 9 | 84 | 245 | 246 |
| 9 | 85 | 247 | 248 |
| 9 | 86 | 249 | 250 |
| 9 | 87 | 251 | 252 |
| 9 | 88 | 253 | 254 |
| 9 | 89 | 255 | 256 |
| 9 | 90 | 257 | 258 |
| 9 | 91 | 259 | 260 |
| 9 | 92 | 261 | 262 |
| 9 | 93 | 263 | 264 |
| 10 | 94 | 265 | 266 |
| 10 | 95 | 267 | 268 |
| 10 | 96 | 269 | 270 |
| 10 | 97 | 271 | 272 |
| 10 | 98 | 273 | 274 |

Table 4 below shows the frequencies of each of the clamp sequences having SEQ ID NOs: 7 to 10 on each of the human chromosomes.

| | Clamp | Clamp | Clamp | Clamp |
|---|---|---|---|---|
| | sequence | sequence | sequence | sequence |
| Chromosome | (SEQ ID | (SEQ ID | (SEQ ID | (SEQ ID |
| number | NO: 7) | NO: 8) | NO: 9) | NO: 10) |
| Chr1 | 27970 | 16945 | 19076 | 12682 |
| Chr2 | 22806 | 13905 | 15603 | 10538 |
| Chr3 | 17925 | 11051 | 12513 | 8269 |
| Chr4 | 14495 | 9131 | 10063 | 6718 |
| Chr5 | 15599 | 9538 | 10604 | 7378 |
| Chr6 | Not checked | Not checked | Not checked | Not checked |
| Chr7 | 18755 | 11155 | 12792 | 8068 |
| Chr8 | 12928 | 7890 | 8812 | 5785 |
| Chr9 | 13452 | 8224 | 9096 | 6158 |
| Chr10 | 14992 | 8969 | 10127 | 6803 |
| Chr11 | 13129 | 7956 | 8913 | 5957 |
| Chr12 | 15808 | 9465 | 10684 | 7182 |
| Chr13 | 7752 | 4848 | 5400 | 3640 |
| Chr14 | 10086 | 5998 | 6654 | 4435 |
| Chr15 | 10659 | 6466 | 7212 | 4794 |
| Chr16 | 13523 | 7870 | 9055 | 5669 |
| Chr17 | 16867 | 9989 | 11071 | 7424 |
| Chr18 | 6556 | 4000 | 4565 | 2949 |
| Chr19 | 15661 | 9239 | 10598 | 6459 |
| Chr20 | 7933 | 4623 | 5301 | 3482 |
| Chr21 | 3450 | 2155 | 2417 | 1590 |
| Chr22 | 6869 | 4121 | 4509 | 3062 |
| ChrX | 13474 | 8043 | 9512 | 5828 |
| ChrY | 1667 | 1214 | 1311 | 817 |
| Total | 302356 | 182795 | 205888 | 135687 |

In certain embodiments, a number of clamps encompassing a highly conserved region are designed by “walking across” the highly conserved region. Each of these clamps can be tested to identify the camp which exhibits maximum experimental ease, accuracy and reproducibility. For the purposes of this invention, the term “walking across” indicates the process of designing a plurality of clamps that bind to an area of interest, wherein each of the plurality of clamps has a specific length, for example, 10-15 bp, each of the plurality of clamps begins at a particular nucleotide of the area of interest and the plurality of clamps as a whole cover the entire are of interest. In general, the clamps are about 15-50 bp, about 15-40 bp, about 15-30 bp, about 16-28 bp, about 17-26 bp, about 18-24 bp, about 19-22 bp, or about 20 bp.

For example, if a SINE is about 300 bp containing a highly conserved region of 50 bp, a plurality of clamps, each of about 10-15 bp, is designed by walking across the highly conserved region of 50 bp. For example, a plurality of clamps of 10 bp are designed based on a highly conserved region of 50 bp by designing clamps that have the sequence of 1-10 bases, 2-11 bases, 3-12 bases, . . . , and 41-50 bases of the highly conserved region. As such, 41 different clamps can be designed and tested to identify the preferred clamp for use in an assay according to the invention.

A person of ordinary skill in the art can design a plurality of clamps of a particular length within an area of interest, and such embodiments are within the purview of the invention.

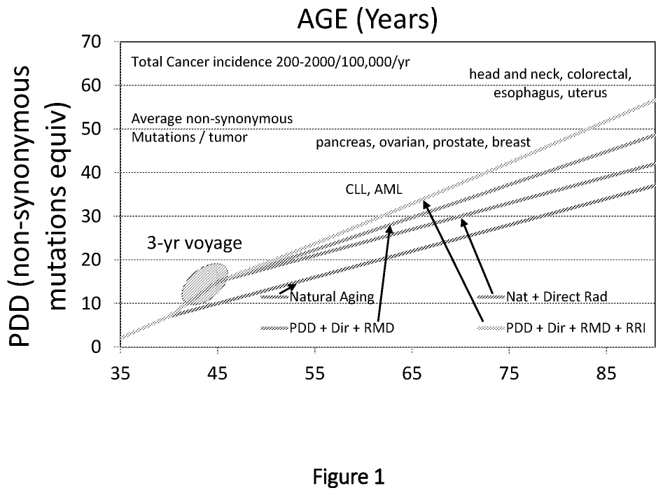

For example, the sequence for Alu is highly conserved, particularly among the first 50 bp, and other sites in the Alu sequence. A sequence alignment between certain Alu sequences is provided in FIG. 1 of Batzer et al. (2002), Nature Reviews Genetics, 3(5):370-379, which is herein incorporated by reference in its entirety. Based on FIG. 1 of Batzer et al., a person of ordinary skill in the art can appreciate that a clamp can be designed to cover the maximum genomic region based on the sequences that correspond to the sequences that are conserved among various versions of Alu sequences. On the other hand, a clamp for an Alu sequence of interest can be designed based on the variation present in the specific Alu sequence of interest.

Additional examples of Alu sequences are provided as SEQ ID NOs: 417 to 478. Conserved domains in these sequences can be determined by a person of ordinary skill in the art to design appropriate clamp sequences and primer sequences.

In a further embodiment, multiple versions of clamps can be used in an assay where the different versions of the clamps are directed to variants of Alu sequences. Therefore, as a whole, the multiple versions of clamps can be used to identify mutations in a region of interest beyond the variability naturally observed in the region of interest.

In preferred embodiments, the clamps are designed based on a region of about 16 to 20 highly conserved bp. The clamp sites also require the presence of suitable 5′ and 3′ primers for the PCR component.

Examples of 14 bp clamps derived from the areas of greatest frequency in Alu, which occur in both mice and humans, are provided in SEQ ID NOs: 7-10. The clamp sequence CCTGTAATCCCAGC (SEQ ID NO: 7) has about 16,000 repeats on chromosome 12; the clamp sequences CTAAAAATACAAAA (SEQ ID NO: 8) and TGCACTCCAGCCTG (SEQ ID NO: 9) each have approximately 10,000 repeats on chromosome 12; and the clamp sequence TCTCAAAAAAAAAA (SEQ ID NO: 10) has approximately 7,000 repeats on chromosome 12. All of these are clamps are suitable for appropriate 3′ and 5′ PCR primers.

In one embodiment, the subject is a mouse and the SINE is B1 having the sequence of SEQ ID NO: 6. An example of the primer pair used in mice with this B1 (SEQ ID NO: 6) as the SINE comprises the primers having the sequences of SEQ ID NOs: 1 and 2 and the target sequence clamp having a sequence selected from SEQ ID NO: 3, 4 and 5.

In another embodiment, the subject is a mouse and the SINE is B1 having the sequence of SEQ ID NO: 281. An example of the primer pair used in mice with this B1 (SEQ ID NO: 281) as the SINE comprises the primers having the sequences of SEQ ID NOs: 479 and 480 and the target sequence clamp having a sequence selected from SEQ ID NO: 3, 4 and 481.

In a further embodiment of the invention, the subject is human and the SINE is Alu.

The step of obtaining a genomic DNA sample from the subject and fragmenting the DNA sample can be performed based on methods well known in the art. Methods and parameters used in fragmenting genomic DNA depend on the size of the genome and the desired average and median size of the fragments. The desired size of the fragments depends on the size of the target SINE, target LINE and/or target sequence within the genome i.e., most of the fragments must allow binding of the primers and the target sequence clamps for the assay to be successful. For example, for a target SINE, target LINE and/or target sequence within the genome of about 100 to 500 bp, the substantial number of DNA fragments is about 800-1500 bp each. For example, in a typical genomic DNA fragment sample, each fragment from at least about 80-99%, about 82-98%, about 84-96%, about 86-94%, about 88-92%, or about 90% of all of the genomic DNA fragments have the desired size. A person of ordinary skill in the art can determine the optimal size of the genomic fragments for a particular assay. Also, the techniques of producing genomic fragments of a desired size are well-known in the art and such embodiments are within the purview of the invention.

A person of ordinary skill in the art would appreciate that since the fragmenting of the genomic DNA is random, a fragment can contain more than one SINE and/or one or more LINE and/or target sequence within the genome. A fragment can also contain only a part of the SINE or part of a LINE and/or target sequence from within the genome. These issues can be addressed by using a large sample and/or running multiple repeats of the assay so that the possible errors are diluted and a more accurate estimation of the mutations is obtained.

Mixing a predetermined number of fragments of the genomic DNA that arise from a predetermined number of genomes with a reagent mixture to produce a reaction mixture is intended to provide the values used in the calculations of the accumulated mutations and/or rate of mutations in the target sequence within the target SINE and/or target LINE and/or the genome. For example, a person of ordinary skill in the art can calculate the accumulated mutations and/or the rage of mutations in the target SINE and/or target LINE and/or target sequence within the genome based on the number of fragments arising from one genome, the number of fragments subjected to dPCR, the number of mutated target sequences as indicated by the positive PCR amplification results and the size and frequency of the target SINE and/or target LINE and/or target sequence within the genome in the genome. Also, based on the size and frequency of the target SINE and/or target LINE and/or target sequence within the genome and the accumulated mutations and/or the rate of mutations in the target SINE and/or target LINE and/or target sequence within the genome, a person of ordinary skill in the art can calculate the accumulated mutations and/or the rate of mutations in the genome.

In addition to the primer pair, the target sequence clamp and the DNA polymerase, the reagent mixture contains reagents for the dPCR. The reagent mixture comprises deoxyribonucleotides (dNTPs), metal ions (for example, Mg²⁻ and Mn²⁻), and a buffer. Additional reagents which may be used in a dPCR reaction are well-known to a person of ordinary skill in the art and such embodiments are within the purview of the invention.

The pair of polymerase chain reaction primers that amplify a target amplicon comprises the target sequence within the target SINE and/or target LINE and/or target sequence within the genome. The primers are designed so that an amplicon is not produced when the target sequence clamp is bound to the target sequence within the target SINE and/or target LINE and/or target sequence within the genome.

Based on a particular target SINE and/or target LINE and/or target sequence within the genome, for example, a target SINE selected from Table 1, a person of ordinary skill in the art can design appropriate primers and the target sequence clamp. For a particular target SINE and/or target LINE and/or target sequence within the genome, person of ordinary skill in the art can test multiple primer pairs and/or target sequence clamps to identify the optimal combination of primers and target sequence clamps and such embodiments are within the purview of the invention.

A target sequence within a target SINE and/or target LINE and/or target sequence within the genome is the sequence to which the target sequence clamp binds. Particularly, the target sequence clamp is complementary to the target sequence within the target SINE and/or target LINE and/or target sequence within the genome.

A wild-type target sequence does not contain any mutations. A mutated target sequence contains one or more point mutations and/or indel mutations. Accordingly, a wild-type target amplicon contains the wild-type target sequence and a mutated target amplicon contains a mutated target sequence.

In one embodiment, the target sequence clamp is designed based on the sequence of the SINE, LINE and/or genomic sequence and is about 15-50 bp, about 15-40 bp, about 15-30 bp, about 16-28 bp, about 17-26 bp, about 18-24 bp, about 19-22 bp, or about 20 bp.

In an embodiment of the invention, the target sequence clamp is designed so that the melting temperature of the target sequence clamp with the target sequence is higher than the temperatures used in the PCR cycle. The higher melting temperature of the clamp ensures that the clamp is bound to the clamp target sequence during the PCR cycles when the clamp is perfectly matched with the target sequence. A mutation in a target sequence reduces the melting temperature of the target sequence clamp with the mutated target sequence and the target sequence clamp is not bound to the mutated target sequence at the temperatures of the PCR cycles, particularly the annealing steps and the amplification steps of the PCR cycles. Therefore, the target sequence clamp prevents PCR amplification of the target amplicon when the amplicon contains the wild-type target sequence and the clamp permits PCR amplification of the target amplicon when the amplicon contains a mutated target sequence.

In another embodiment of the invention, the target sequence clamp comprises xenonucleotide (XNA). A variety of XNA are known in the art. The target sequence XNA clamp also suppresses PCR amplification of the amplicons containing wild-type clamp target sequences and allows selective PCR amplification of only the amplicons containing mutated target clamp sequences. XNA, for example, can contain an amino acid linkages rather than a phosphate between bases, which causes it to bind tightly with the wild-type clamp target sequence and reduces hydration and heat instability. Therefore, a target XNA sequence clamp does not melt off the wild-type clamp target sequence at the usual PCR temperatures when the match is perfect.

In a further embodiment, a target XNA clamp of about 13-20 bp is used. In other embodiments, the XNA clamp is about 15-50 bp, about 15-40 bp, about 15-30 bp, about 16-28 bp, about 17-26 bp, about 18-24 bp, about 19-22 bp, or about 20 bp. When a 13-20-bp XNA is used, a single-point mutation in the target sequence within the target SINE and/or target LINE and/or target sequence within the genome lowers the melting point between the two binding sequences by 15-20° C. Indel mutations lower the melting point between a target XNA sequence clamp and the wild-type clamp target sequence by more than 15-20° C. Because the mutated target sequence does not bind the target XNA sequence clamp, only the amplicons containing the mutated target sequences are amplified during PCR.

Accordingly, when the target sequence clamp binds to the wild-type target sequence within the target SINE and/or target LINE and/or target sequence within the genome the target sequence clamp prevents the PCR amplification of the target amplicons that have the target wild-type sequence within the SINE. In contrast, when the target sequence contains a mutation, the target sequence clamp cannot bind to the target sequence, which allows the PCR amplification of the target amplicons that have the target mutated sequence within the SINE, LINE and/or target genomic sequence.

dPCR, as used in the claimed invention, refers to a PCR where the PCR reaction is carried out as a single reaction within a sample; however, the sample is separated into a large number of partitions and the reaction is carried out in each partition individually and separately from the other partitions. dPCR involves identification of the amplification of the target amplicons in each of the large number of partitions. dPCR enables precise and highly sensitive quantification of nucleic acids. An overview of dPCR is provided by Baker (2012), the contents of which are incorporated herein in their entirety.

In one embodiment of the invention, the dPCR used in the assay is droplet digital PCR (ddPCR). In ddPCR, a PCR sample is partitioned into a large number of droplets, for example, 20,000 droplets, using water-oil emulsion droplet technology. After amplification, droplets containing the target sequence are detected by fluorescence and scored as positive, and droplets without fluorescence are scored as negative. Poisson statistical analysis of the numbers of positive and negative droplets yields absolute quantitation of the target sequences. An overview of ddPCR is provided by Hundson et al. (2011), the contents of which are incorporated herein in their entirety.

When the dPCR results are obtained, the number of accumulated mutations per genome in the target sequence within the target SINE and/or target LINE and/or target sequence within the genome is calculated based on the number of fragments of the genomic DNA that arise from one genome and the number of fragments per genome that comprise the target amplicons having the target mutated sequence within the target SINE and/or target LINE and/or target sequence within the genome. As discussed above, the presence of the target mutated sequence within the target SINE and/or target LINE and/or target sequence within the genome is indicated by the positive PCR amplification in the dPCR.

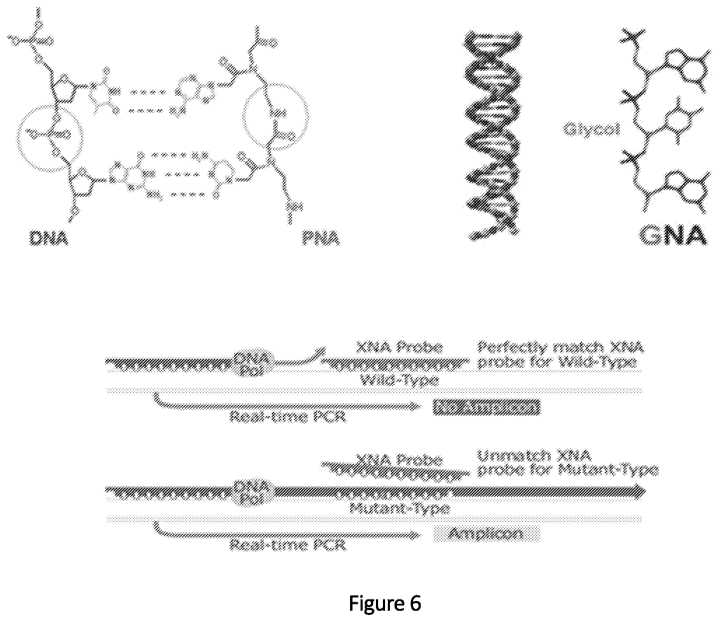

In standard dPCR or a ddPCR mix, the assay of the invention enables the detection of 1-2 mutant DNA fragments in a pool of 100,000 wild-type amplicons (FIG. 6). The use of the target sequence clamp in combination with dPCR provides an extraordinary mutation screening method. For example, in a 45-min cycle of a ddPCR (BioRad QX200 AutoDG ddPCR, Hercules, Calif.) over 10⁶ DNA fragments can be analyzed in each of the 8 channels for the presence of mutations. Since about 10% of those DNA fragments likely contain Alu, ˜10⁵ Alu are analyzed at one fragment per well or drop. Using 100 or 1,000 fragments per drop instead of ˜1-2 per drop improves the screening of Alu fragments by 2 or 3 orders of magnitude (up to 10⁻⁸ Alu/channel).

As such, an embodiment of the invention provides a method of estimating the accumulated mutations in a target sequence within a target SINE and/or target LINE. The number of accumulated mutations in a target sequence within a target SINE and/or target LINE and/or target sequence within the genome can be used to determine the rate of mutations in the target sequence within the target SINE and/or target LINE and/or target sequence within the genome. For example, the accumulated mutations in the target sequence within the target SINE and/or target LINE and/or target sequence within the genome can be determined at two time points and the rate of mutations can be calculated based on the difference in the number of accumulated mutations in the target sequence within the target SINE and/or target LINE and/or target sequence within the genome and the duration between the two time points.

In another embodiment, a first sample is obtained from the subject at Time 1 and a second sample is obtained from the subject at Time 2. The accumulated mutations in the target sequence within the target SINE and/or target LINE and/or target sequence within the genome are estimated in the first and the second samples according to the clamp/dPCR combination assay and the rate of mutations in the target sequence within the target SINE and/or target LINE and/or target sequence within the genome can be calculated based on the difference in the number of accumulated mutations in the target sequence within the target SINE and/or target LINE and/or target sequence within the genome and the duration between Time 1 and Time 2.

In a specific embodiment, a sample is obtained from the subject at birth. This sample provides accumulated mutations in the target sequence within the target SINE and/or target LINE and/or target sequence within the genome at birth, which can be considered as baseline mutations or the state of no mutations. The accumulated mutations estimated in a sample obtained from the subject at a later time can be compared to the baseline mutations or the state of no mutations.

Accordingly, an embodiment of the invention provides a method for calculating the rate of mutations in a target sequence within a target SINE and/or target LINE and/or target sequence within the genome in a subject. The method comprises the steps of:

a) according to the clamp/dPCR combination assay, determining the number of accumulated mutations in the target sequence within the target SINE and/or target LINE and/or target sequence within the genome in a first sample obtained from the subject at a first time point,

b) according to the clamp/dPCR combination assay, determining the number of accumulated mutations in the target sequence within the target SINE and/or target LINE and/or target sequence within the genome in a second sample obtained from the subject at a second time point,

c) calculating the rate of mutations in the target sequence within the target SINE and/or target LINE and/or target sequence within the genome in the subject based on the difference between the number of accumulated mutations in the target sequence within the target SINE and/or target LINE and/or target sequence within the genome in the subject at the first time point and the second time point and the duration between the first time point and the second time point.

The number of accumulated mutations and/or the rate of mutations in the target sequence within the target SINE and/or target LINE and/or target sequence within the genome can be used to estimate the accumulated mutations and/or the rate of mutations in the genome of a subject. For example, the number of accumulated mutations in the genome of a subject can be calculated based on the frequency of occurrence of a target SINE and/or target LINE and/or target sequence within the genome throughout the genome and the number of accumulated mutations in the target sequence within the target SINE and/or target LINE and/or target sequence within the genome.

Similarly, the rate of mutations in a target sequence within a target SINE and/or target LINE and/or target sequence within the genome can be used to estimate the rate of mutations in the genome of the subject. For example, the rate of accumulated mutations in the genome of the subject can be calculated based on the frequency of occurrence of the target SINE and/or target LINE and/or in the genome and the rate of mutations in the target sequence within the target SINE.

As such, an embodiment of the invention provides a method for determining accumulated mutations and/or the rate of the mutations in a target sequence within a target SINE and/or target LINE and/or the genome of the subject.

Accumulated mutations and/or the rate of mutations typically increase with age. For example, the number of accumulated mutations and/or the rate of mutations in a chronologically older subject are typically higher than the corresponding values in a chronologically younger subject. Also, different individuals age at a different rates, i.e., the accumulated mutations and/or the rate of mutations in two individuals of the same age can be different. For example, individuals exposed to higher levels of mutagens like carcinogens, mutagenic chemicals, radiation, stress, etc. typically have more accumulated mutations and/or a higher rate of mutations compared to individuals not exposed to such mutagens or exposed to relatively lower levels of mutagens.

A standard scale for genomic age for a particular species can be determined based on average accumulated mutations and/or the average rate of mutations in different groups of individuals of varying ages that are living under the conditions of exposure to only natural mutagens and/or the conditions of minimal exposure to man-made mutagens.

For the purpose of the invention, the phrase “the conditions of exposure to only natural mutagens” indicates exposure to only unavoidable natural mutagens, for example, cosmic radiation, ultraviolet rays from the sun, mutagens that may be naturally (i.e., without interference from humans) present in soil, air, water, and food or other environmental factors. Additional examples of unavoidable natural mutagens can be readily envisioned by a person of ordinary skill in the art.

For example, an individual living in the conditions of exposure to only natural mutagen is living in conditions that are free from:

a) exposure to man-made mutagens, such as synthetic carcinogens, synthetic pollutants, radiation from man-made sources, etc., and

b) avoidable/unnecessary exposure to natural mutagens, for example, smoking, using tobacco and other avoidable/unnecessary exposure to natural carcinogens.

Similarly, for the purpose of the invention, the phrase “the conditions of minimal exposure to man-made mutagens” indicates minimal exposure to unavoidable natural mutagens (discussed above) and minimal exposure to man-made mutagens, such as synthetic carcinogens, synthetic pollutants and radiation from man-made sources. The conditions of minimal exposure to man-made mutagens are also free from avoidable/unnecessary exposure to natural mutagens, for example, smoking, using tobacco and other avoidable/unnecessary exposure to natural carcinogens.

An example of an individual living under the conditions of exposure to only natural mutagens and/or the conditions of minimal exposure to man-made mutagens is an individual living in the countryside. Because of the industrialized lifestyle of almost everyone in the world, it is very difficult and almost impossible to find individuals living under the conditions of exposure to only natural mutagens. Therefore, the standard scale for the genomic age for a particular species can be determined based on the average accumulated mutations and/or the average rate of mutations in different groups of individuals of varying ages that are living under the conditions of minimal exposure to man-made mutagens.

Accordingly, a standard scale for the genomic age of humans can be produced by determining the average accumulated mutations and/or the average rate of mutations in humans of varying ages that live in the conditions of minimal exposure to man-made mutagens, for example, people living in the countryside. Such a scale of genomic age can be used to determine the genomic age of an individual based on the individual's accumulated mutations and/or rate of mutations in the genome.

The exposure to avoidable/unnecessary natural mutagens and/or the exposure to man-made mutagens typically increase the accumulated mutations and/or the rate of mutations in the genome of a subject. For example, a person living in the countryside typically has fewer accumulated mutations and/or a lower rate of mutations compared to a person living in a city, particularly a polluted city. Therefore, a person of a particular chronological age living in the countryside typically has a lower genomic age compared to the genomic age of a person of the same chronological age living in a city.

Accordingly, the clamp/dPCR combination assay for determining the accumulated mutations and/or the rate of the mutations in a target sequence in a target SINE, target LINE and/or the genome of a subject can be used to determine the genomic age of the subject. The method comprises the steps of:

a) preparing a standard genomic age scale for individuals belonging to the species of the subject and living under the conditions of exposure to only natural mutagens or the conditions of minimal exposure to man-made mutagens, or obtaining a pre-determined standard genomic age scale for the species of the subject,

b) determining the accumulated mutations and/or the rate of mutations in the subject according to the clamp/dPCR combination assay, and

c) estimating the genomic age of the subject based on the comparison of the accumulated mutations and/or the rate of mutations in the subject with the standard scale for the genomic age of the subject.

Similar to increasing age, a subject having cancer or having a higher risk of developing cancer exhibits an increase in the accumulated mutations and/or the rate of mutations in a target sequence in a target SINE, target LINE and/or the genome. For example, a person of a particular chronological age having more accumulated mutations and/or a higher rate of mutations in a target sequence in a target SINE, target LINE and/or the genome is at a higher risk of developing cancer compared to a person of the same chronological age who has relatively fewer accumulated mutations and/or a lower rate of mutations in a target sequence in a target SINE, target LINE and/or the genome.

Also, chronologically older individuals are at a higher risk of developing cancer because the accumulated mutations and/or the rate of mutations in a target sequence in a target SINE, target LINE and/or the genome are typically higher in chronologically older individuals compared to the corresponding values in chronologically younger individuals.

Accordingly, an embodiment of the invention provides a method of identifying a higher risk of cancer development in a subject based on the accumulated mutations and/or the rate of mutations in a target sequence in a target SINE, target LINE and/or the genome of the individual and a standard scale of cancer risk in the species to which the subject belongs.

The standard scale of cancer risk indicates the risk of cancer in a subject based on the accumulated mutations and/or the rate of mutations in a target sequence in a target SINE, target LINE and/or the genome of the subject. For example, more accumulated mutations and/or a higher rate of mutations in a target sequence in a target SINE, target LINE and/or the genome in a subject indicates a higher risk of cancer development in the subject compared to an individual of the same chronological age as the subject and having relatively fewer accumulated mutations and/or a lower rate of mutations in a target sequence in a target SINE, target LINE and/or the genome.

A standard scale of the cancer risk for a species, for example, humans, can be produced by determining the average accumulated mutations and/or the average rate of mutations in a target sequence in a target SINE, target LINE and/or the genome in individuals of varying ages that are free from cancer and/or are known to have a low risk of cancer development. The standard scale of cancer risk can be used to determine the risk of cancer development in a subject based on the subject's accumulated mutations and/or rate of mutations in a target sequence in a target SINE, target LINE and/or the genome and the subject's chronological age. As such, the standard scale of cancer risk in the species indicates, at increasing chronological age, the average accumulated mutations and/or the average rate of mutations in the target sequence in the target SINE, target LINE and/or the genomes of individuals of varying ages that belong to the species and are free from cancer and/or are known to have a low risk of cancer development.

Accordingly, an embodiment of the invention provides a method for determining the risk of cancer development of a subject based on accumulated mutations and/or rate of mutations in a target sequence in a target SINE, target LINE and/or the genome of the subject. The method comprises the steps of:

a) preparing a standard scale for cancer risk by determining the average accumulated mutations and/or the average rate of mutations in a target sequence in a target SINE, target LINE and/or the genome in individuals of varying ages that are free from cancer and/or are known to have a low risk of cancer development, or obtaining a pre-determined standard scale of cancer risk,

b) determining the accumulated mutations and/or the rate of mutations in the target sequence in the target SINE, target LINE and/or the genome in the subject, and

c) estimating the risk of cancer development of the subject based on the comparison of the accumulated mutations and/or the rate of mutations in the target sequence in the target SINE, target LINE and/or the genome of the subject with the standard scale of cancer risk.

The step of estimating the risk of cancer development of the subject based on the comparison of the accumulated mutations and/or the rate of mutations in the target sequence in the target SINE, target LINE and/or the genome of the subject with the standard scale can be:

a) identifying the subject as having a higher risk of cancer development if the accumulated mutations and/or the rate of mutations in the target sequence in the target SINE, target LINE and/or the genome of the subject are higher than the corresponding values in the standard scale of cancer risk, or

b) identifying the subject as having a lower risk or no risk of cancer development if the accumulated mutations and/or the rate of mutations in the target sequence in the target SINE, target LINE and/or the genome of the subject are lower than or equal to the corresponding values in the standard scale of cancer risk.

Every individual always carries some risk of cancer development. For example, spontaneous oncogenic mutations occur even among individuals living with minimal exposure to mutagens. Therefore, for the purpose of this invention, a higher risk of cancer development of a subject refers to a higher risk of cancer development compared to the risk of cancer development in the population of the same chronological age as the subject that is free from cancer and/or is known to have a low risk of cancer development. Similarly, a lower risk of cancer development of a subject refers to a lower risk of cancer development compared to the risk of cancer development in the average population of the same chronological age as the subject that is free from cancer and/or is known to have a low risk of cancer development.

If a subject is identified as having a higher risk of cancer development, enhanced screening for cancer can be administered to the subject for early detection and treatment of cancer. As is well-established in the art, early detection and treatment of cancer typically results in cancer-free survival. Therefore, administering enhanced screening to a subject based on the subject's identification as having a higher risk of cancer development ensures that the cancer, if developed, is identified during the early stages, thereby increasing the chances of cancer-free survival of the subject.

Enhanced screening for cancer indicates that the cancer screening is administered more frequently than recommended for a healthy individual. For example, if recommended screening frequency for cancer for a healthy individual is once a year, an individual identified as having a higher risk of cancer development can be screened every six months. Recommended cancer screening schedules for various cancers and the modifications which can be done to the recommended schedules to produce an enhanced screening schedule are well-known to a person of ordinary skill in the art and such embodiments are within the purview of the invention.

Also, if a subject is identified as having a higher risk of cancer development, lifestyle changes can be recommended to the subject to reduce the risk of cancer development. Non-limiting examples of lifestyle changes which can reduce the risk of cancer development include cessation of smoking, reducing the exposure to a known carcinogen, or changing a profession or job which poses increased exposure to a particular carcinogen. Additional examples of lifestyle changes which can reduce the risk of cancer development are well-known to a person of ordinary skill in the art and such embodiments are within the purview of the invention.

If a subject is identified as having a low risk or no risk of cancer development, enhanced screening for cancer is withheld from the subject and, optionally, routine screening is administered. Withholding enhanced screening for cancer from a subject based on the subject's identification as having a lower risk or no risk of cancer development ensures that the subject does not receive any unnecessary cancer screening. Avoiding unnecessary cancer screening may be significant because sometimes the cancer screening itself uses mutagens, for example, x-rays for the identification of breast cancer.

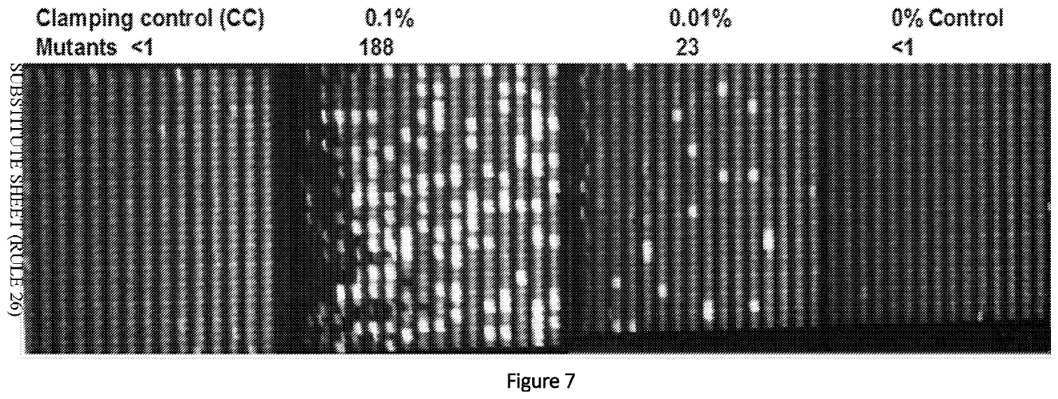

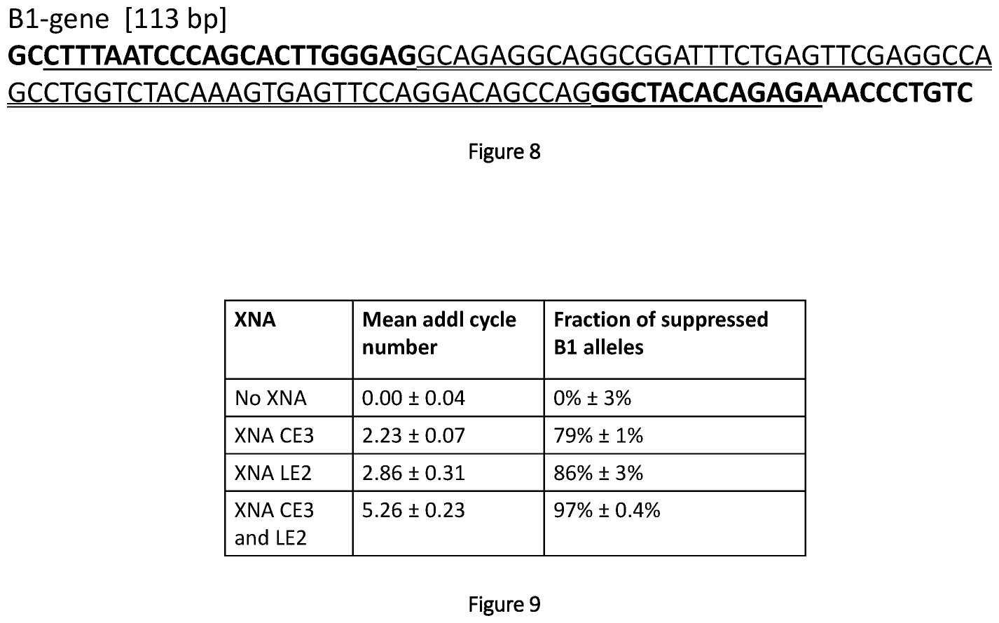

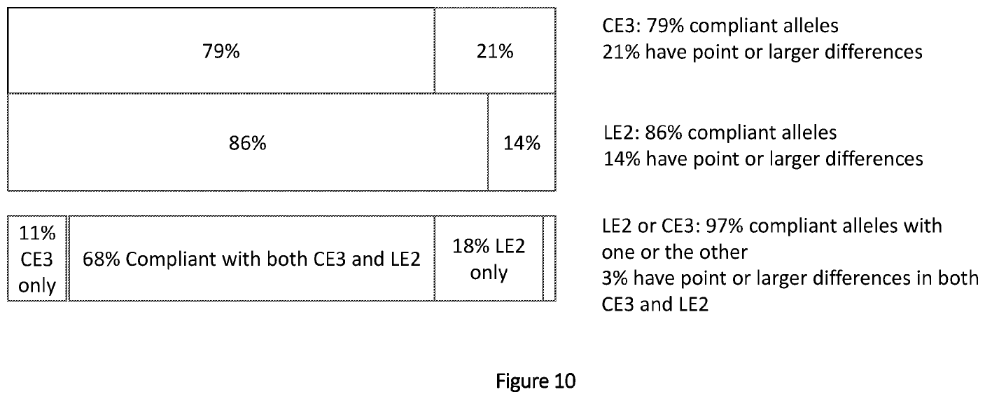

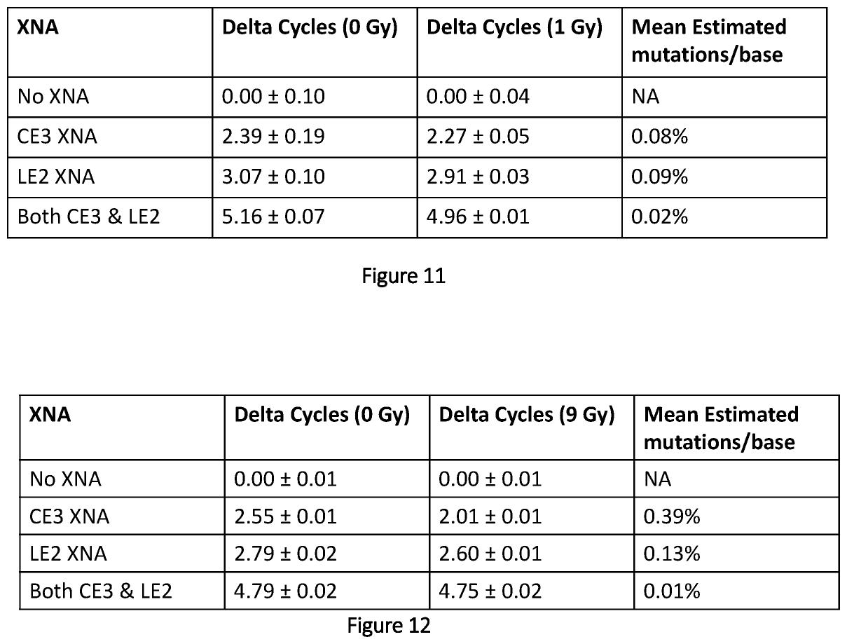

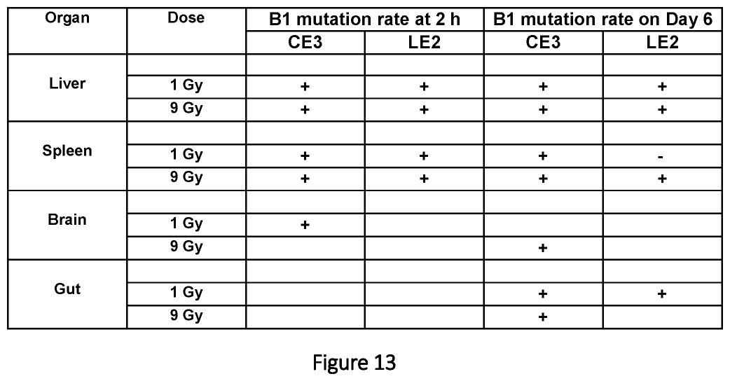

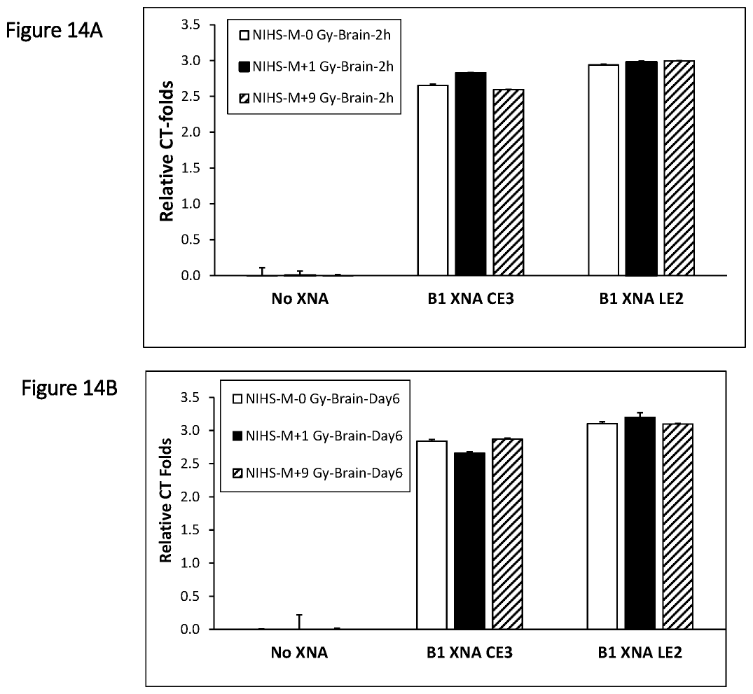

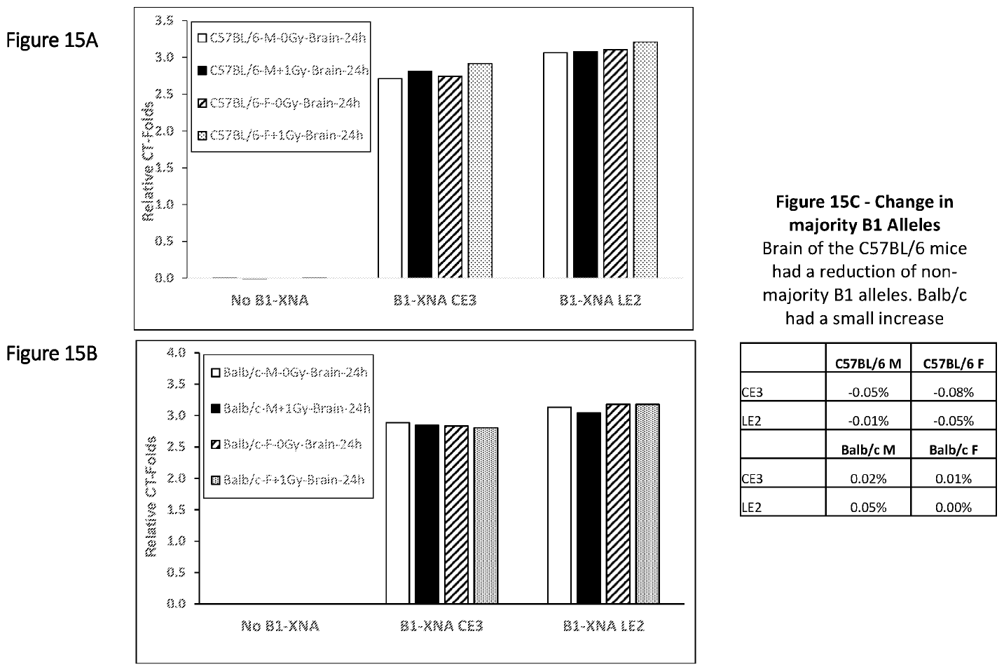

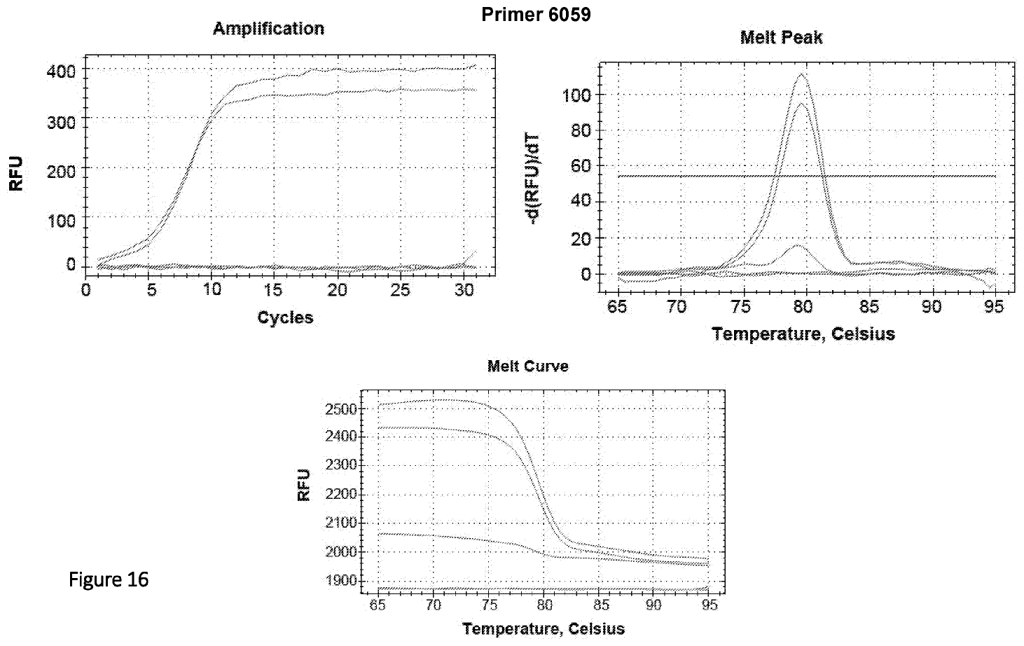

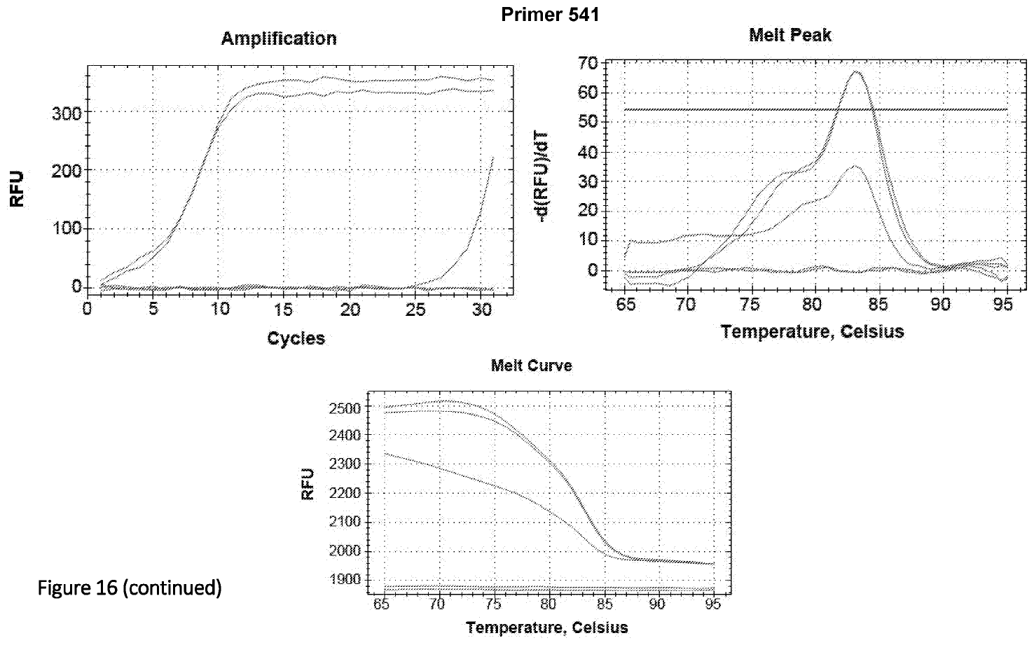

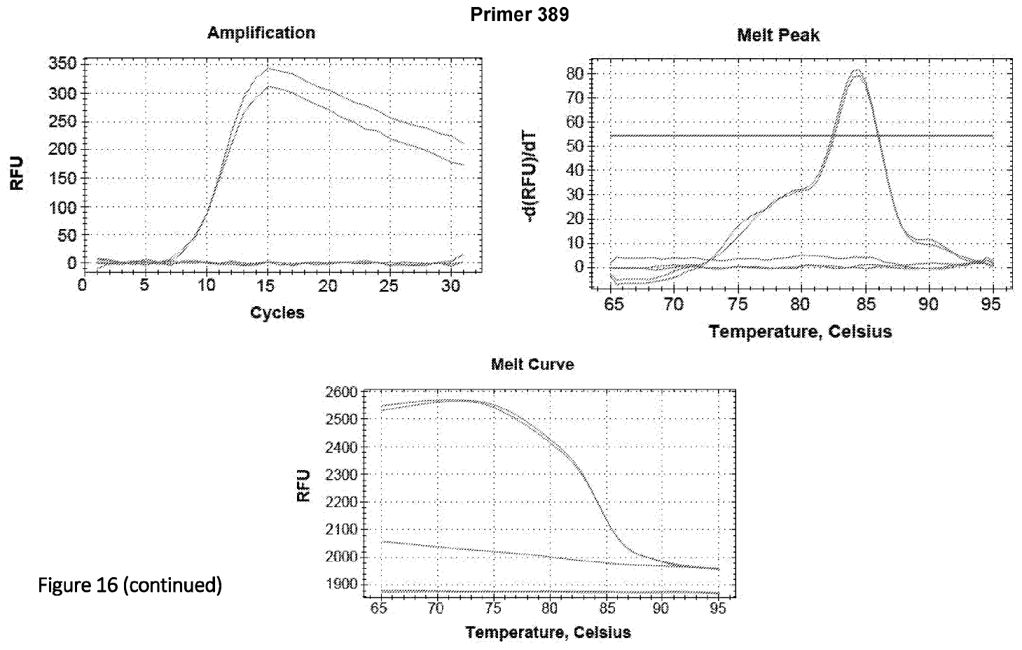

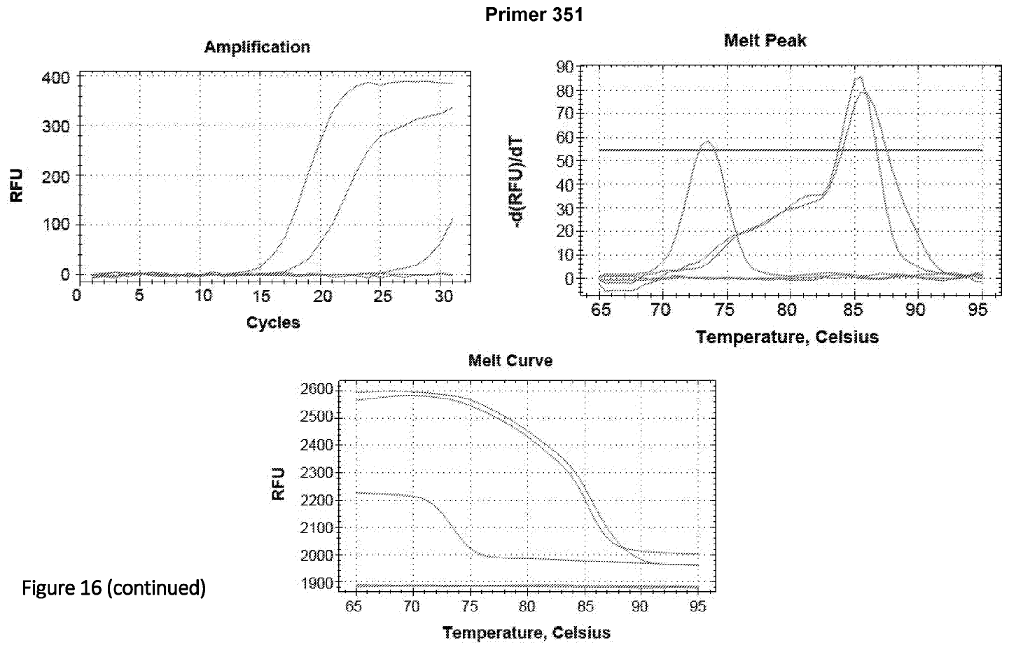

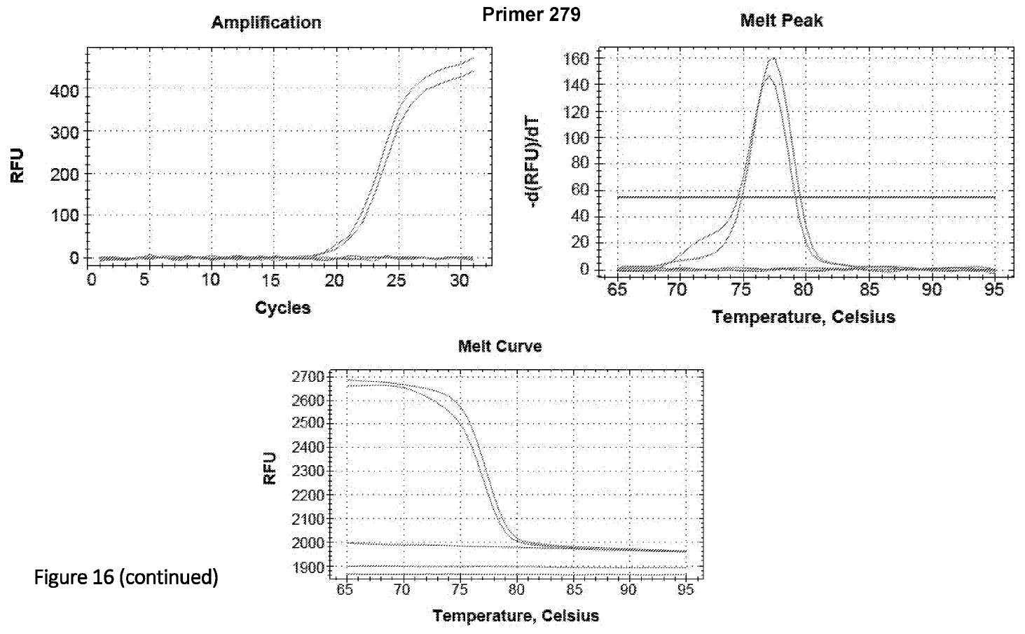

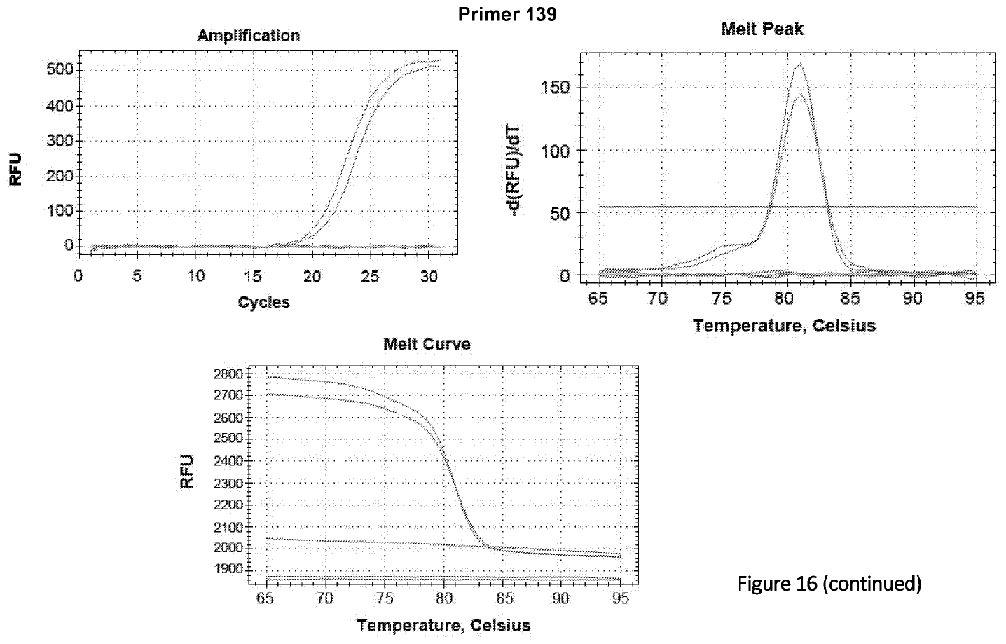

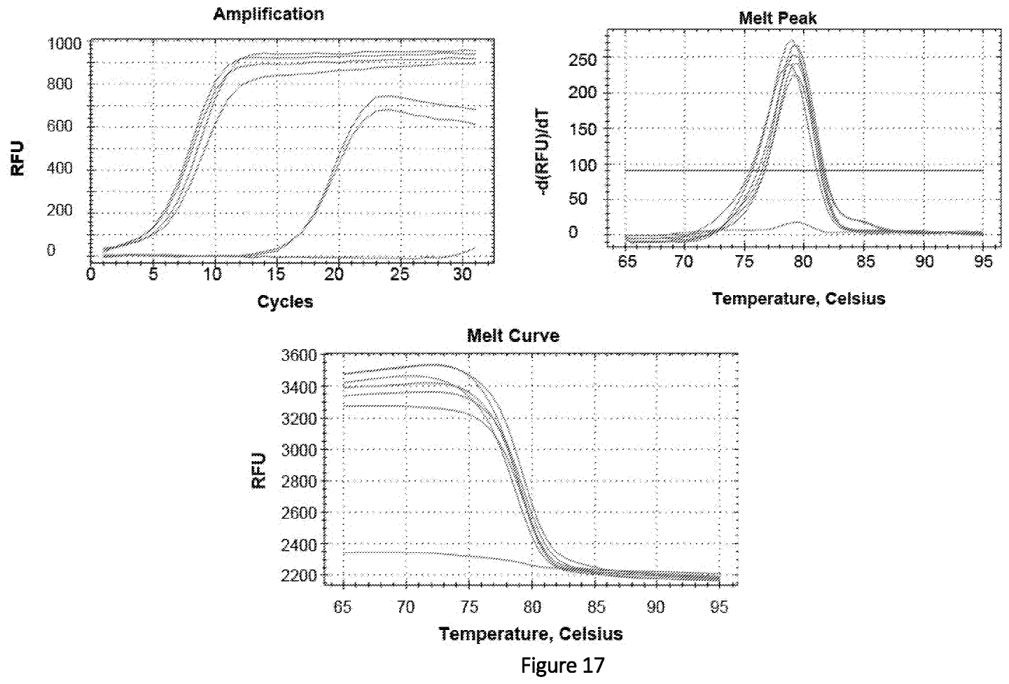

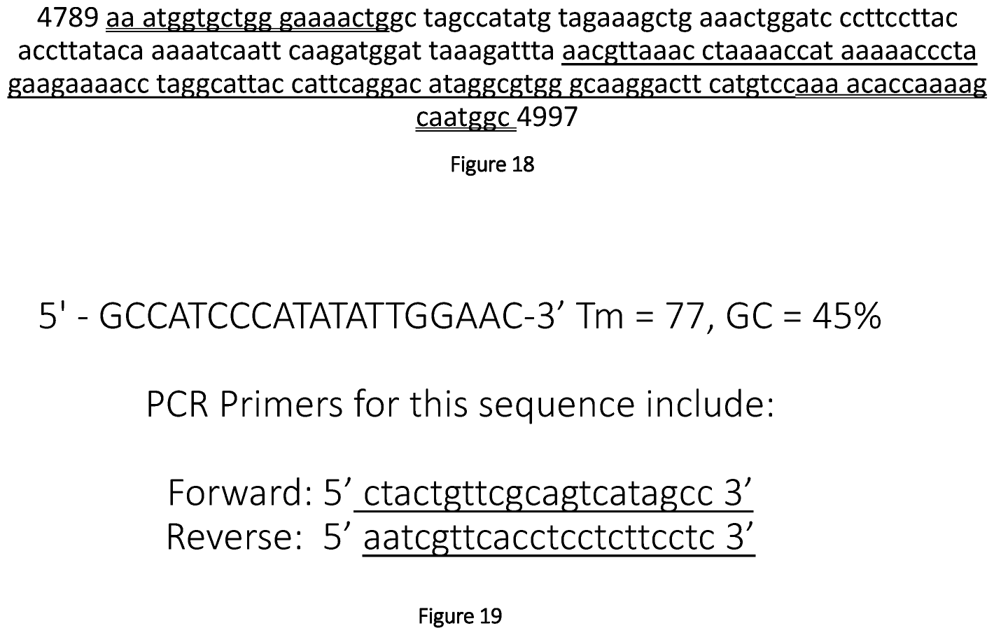

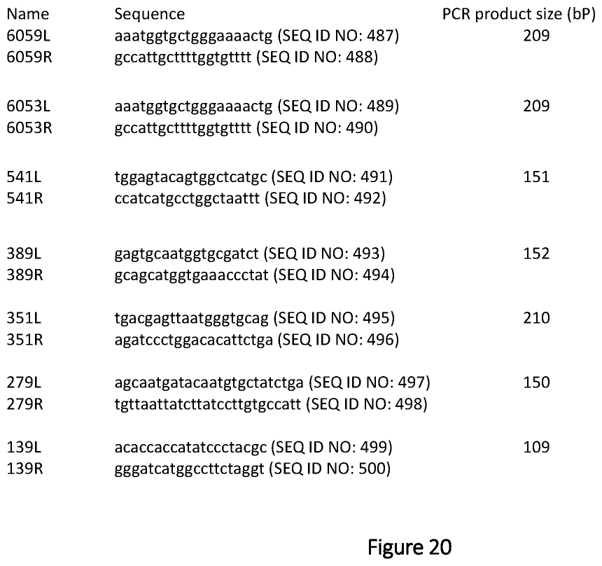

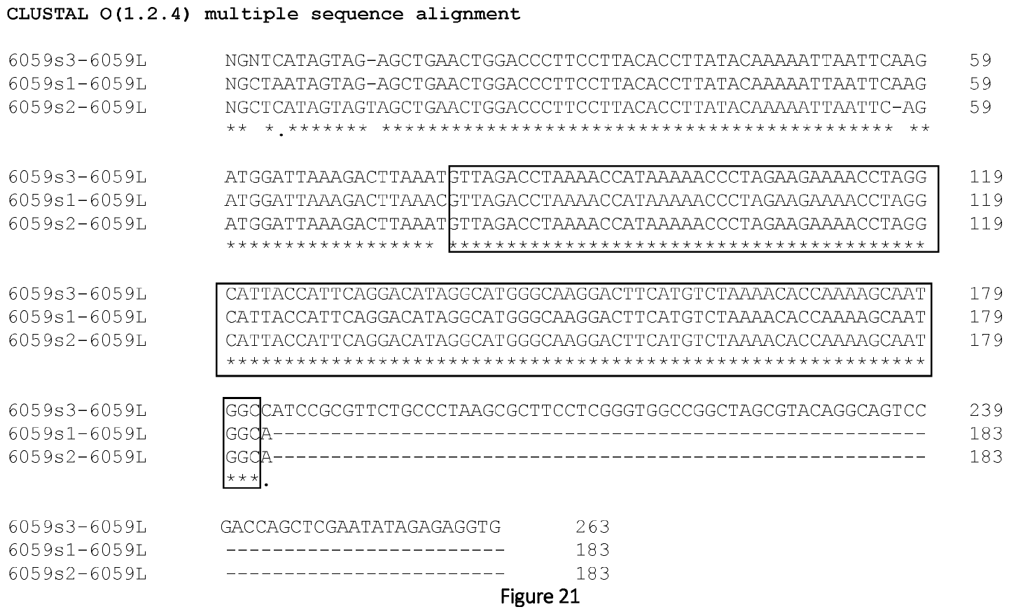

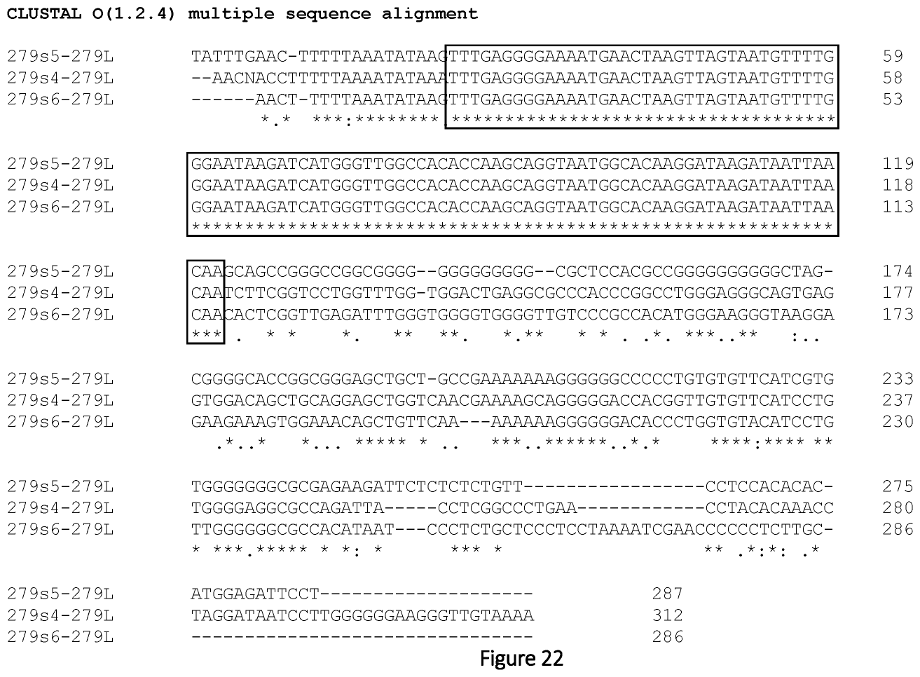

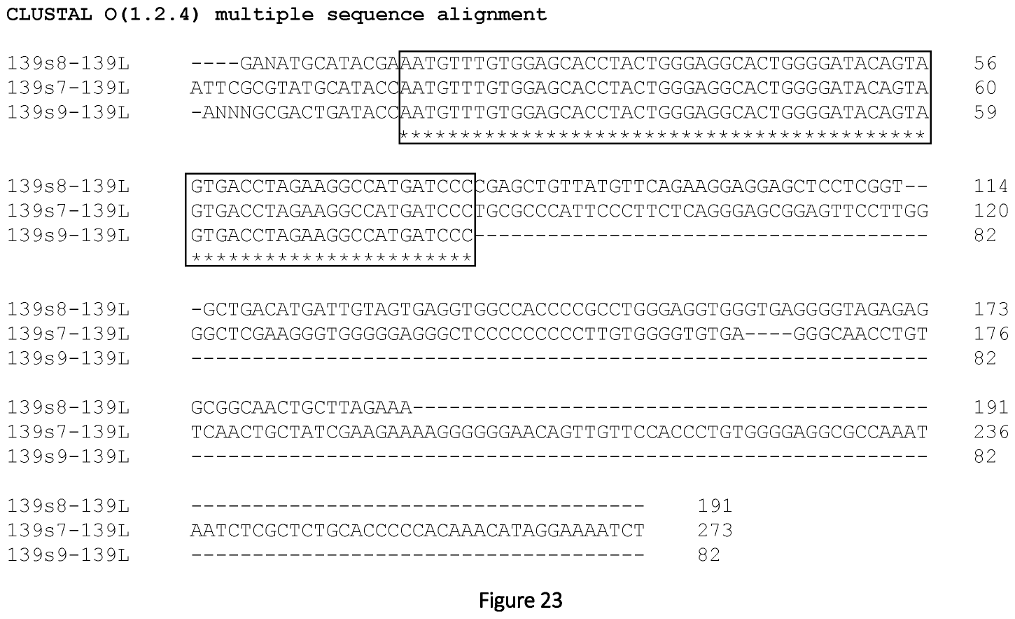

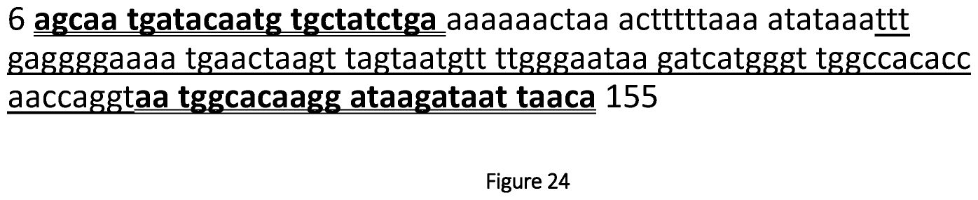

Personal lifestyle and environmental exposures affect a subject's risk for cancer. These factors are extrinsic to a subject's inherited genetics. For example, smoking causes as much as a 10-fold increased rate of accumulation of pulmonary epithelial mutations. Therefore, an embodiment of the invention provides a tissue; or organ-specific prediction of the risk of cancer development. Most solid tumors in adults have 33 to 66 genes with subtle somatic mutations expected to alter their proteins (FIG. 7); the exceptions include lung cancers (smoking; ˜150) and melanomas (sun exposure; ˜135). Thus, tissues naturally exposed to powerful carcinogens appear to require more mutations, suggesting they are relatively resistant to cancer. The level of passengers/silent mutations can be established for cancer types in specific tissues or organs and this level can be used to predict the risk of organ-specific cancer development.

Accordingly, a further embodiment of the invention provides a method of identifying a higher risk of cancer development in a tissue or organ of a subject based on the accumulated mutations and/or the rate of mutations in a target sequence in a target SINE, target LINE and/or the genome of the cells of the tissue or organ.

A standard scale of the cancer risk for a tissue or organ in a species, for example, breast cancer in humans, can be produced by determining the average accumulated mutations and/or the average rate of mutations in a target sequence in a target SINE, target LINE and/or the genome in the cells of the tissue or organ from individuals of varying ages that are free from cancer and/or are known to have a low risk of cancer development. The standard scale of cancer risk for a tissue or organ can be used to determine the risk of cancer development in the tissue or organ of a subject based on the subject's accumulated mutations and/or rate of mutations in a target sequence in a target SINE, target LINE and/or the genome in the cells of the tissue or organ and the subject's chronological age. As such, a standard scale of cancer risk for a tissue or organ in a species indicates, at increasing chronological ages, the average accumulated mutations and/or the average rate of mutations in a target sequence in a target SINE, target LINE and/or the genomes of the cells in the tissues or organs of individuals of varying ages that belong to the species and are free from cancer and/or are known to have a low risk of cancer development in the tissue or organ.

Non-limiting examples of the tissue or organ which can be used in the methods of the invention include placenta, brain, eyes, pineal gland, pituitary gland, thyroid gland, parathyroid glands, thorax, heart, lung, esophagus, thymus gland, pleura, adrenal glands, appendix, gall bladder, urinary bladder, large intestine, small intestine, kidneys, liver, pancreas, spleen, stoma, ovaries, uterus, testis, skin, blood or buffy coat sample of blood. Additional examples of organs and tissues are well-known to a person of ordinary skill in the art and such embodiments are within the purview of the invention.

In certain embodiments, the methods of current invention are practiced to determine the risk of cancer, wherein the cancer is selected from acute lymphoblastic leukemia, acute myeloid leukemia, adrenocortical carcinoma, AIDS-related cancers, AIDS-related lymphoma, anal cancer, appendix cancer, astrocytoma, cerebellar cstrocytoma, basal cell carcinoma, bile duct cancer, extrahepatic bladder cancer, bladder cancer, bone cancer, osteosarcoma and malignant fibrous histiocytoma, brain stem glioma, brain tumor, central nervous system embryonal tumors, cerebral astrocytoma/malignant glioma, ependymoblastoma, medulloblastoma, medulloepithelioma, pineal parenchymal tumors of intermediate differentiation, supratentorial primitive neuroectodermal tumors and pineoblastoma, visual pathway and hypothalamic glioma, brain and spinal cord tumors, breast cancer, bronchial tumors, Burkitt lymphoma, carcinoid tumor, gastrointestinal cancer, carcinoma of the head and neck, central nervous system embryonal tumors, central nervous system lymphoma, cervical cancer, chordoma, chronic lymphocytic leukemia, chronic myelogenous leukemia, chronic myeloproliferative disorders, colorectal cancer, cutaneous T-cell lymphoma, embryonal tumors, endometrial cancer, ependymoblastoma, ependymoma, esophageal cancer, Ewing family of tumors, extracranial germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), extracranial germ cell tumor, germ cell tumor, extragonadal germ cell tumor, ovarian cancer, gestational trophoblastic tumor, glioma, brain stem glioma, hairy cell leukemia, head and neck cancer, hepatocellular (liver) cancer, Hodgkin's lymphoma, hypopharyngeal cancer, hypothalamic and visual pathway glioma, intraocular melanoma, islet cell tumors (endocrine pancreas), Kaposi sarcoma, kidney (renal cell) cancer, kidney cancer, laryngeal cancer, chronic lymphocytic leukemia, chronic leukemia, myelogenous leukemia, lip and oral cavity cancer, lung cancer, non-small cell lung cancer, small cell lymphoma, AIDS-related lymphoma, cutaneous T-cell lymphoma, non-Hodgkin's lymphoma, macroglobulinemia, Waldenström macroglobulinemia, malignant fibrous histiocytoma of bone and osteosarcoma, medulloblastoma, medulloepithelioma, melanoma, intraocular Merkel cell carcinoma, mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, myelogenous leukemia, multiple myeloproliferative disorders, nasal cavity and paranasal sinus cancer, nasopharyngeal cancer, neuroblastoma, non-small cell lung cancer, oral cancer, oral cavity cancer, lip and oropharyngeal cancer, osteosarcoma and malignant fibrous histiocytoma of bone, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal pelvis and ureter raner, transitional cell cancer, respiratory tract carcinoma involving the NUT gene on chromosome 15, retinoblastoma, rhabdomyosarcoma, salivary gland cancer, sarcoma, soft tissue sarcoma, uterine Sézary syndrome, skin cancer (nonmelanoma), skin carcinoma, small cell lung cancer, small intestine cancer, squamous cell carcinoma, squamous neck cancer with occult primary cancer, supratentorial primitive neuroectodermal tumors, T-cell lymphoma, testicular cancer, throat cancer, thymoma and thymic carcinoma, thyroid cancer, transitional cell cancer of the renal pelvis and ureter, gestational trophoblastic tumor, carcinoma of unknown primary site, urethral cancer, uterine cancer, endometrial uterine sarcoma, vaginal cancer, visual pathway and hypothalamic glioma, vulvar cancer, and Wilms' tumor.

Accordingly, an embodiment of the invention provides a method for determining the risk of cancer development of a tissue or organ of a subject based on accumulated mutations and/or rate of mutations in a target sequence in a target SINE, target LINE and/or the genome in the cells of the tissue or organ of the subject. The method comprises the steps of:

a) preparing a standard scale for the cancer risk for the tissue or organ by determining the average accumulated mutations and/or the average rate of mutations in a target sequence in a target SINE, target LINE and/or the genome in the cells of the tissue or organ in individuals of varying ages that are free from cancer and/or are known to have a low risk of cancer development, or obtaining a pre-determined standard scale of cancer risk,

b) according to the clamp/dDNA combination assay of the invention, determining the accumulated mutations and/or the rate of mutations in the target sequence in the target SINE, target LINE and/or the genome in the cells of the tissue or organ of the subject, and

c) estimating the risk of cancer development of the tissue or organ of the subject based on the comparison of the accumulated mutations and/or the rate of mutations in the target sequence in the target SINE, target LINE and/or the genome in the cells of the tissue or organ of the subject with the standard scale of cancer risk for the tissue or organ.

A person of ordinary skill in the art would appreciate that the steps described above for estimating the risk of cancer development in the subject and the steps taken after a subject is identified as having a higher or lower risk of cancer development are relevant to cancer development of a tissue or organ and are within the purview of the invention as it relates to the estimation of a risk of cancer development of a tissue or organ.

Lifestyle changes are prescribed by medical professionals as means of improving overall health and well-being of humans, including decreasing the chances of cancer development or slowing down the rate of aging. An embodiment of the invention provides a method of determining whether a particular lifestyle change or a combination of lifestyle changes effectively reduced the risk of cancer development in a subject or effectively slowed down the rate of aging.

For example, a person's risk of cancer development, such as a cancer of a particular tissue or organ, can be determined before and after a lifestyle change is initiated. Similarly, a person's rate of aging, for example, rate of increase in the genomic age, can be determined before and after a lifestyle change is initiated.

Non-limiting examples of lifestyle changes that are recommended for reducing the risk of cancer and/or slowing down the rate of aging include weight loss, cessation of smoking, limiting the exposure to a known carcinogen, change of a profession or job to avoid exposure to a particular carcinogen, dietary changes, etc. Additional examples of lifestyle changes that can be prescribed to reduce the risk of cancer development and/or slow down the rate of aging are well-known to a person of ordinary skill in the art and such embodiments are within the purview of the invention.

If the risk of cancer development and/or the rate of aging are reduced, indicated, for example, by a reduced rate of mutations in a target sequence in a target SINE, target LINE and/or the genome of the subject, the lifestyle change is considered to be successful in achieving the intended goal.

On the other hand, if the risk of cancer development and/or the rate of aging are not reduced or are not reduced to the desired extent, indicated, for example, by no reduction or lack of reduction to the preferred extent in the rate of mutations in a target sequence in a target SINE, target LINE and/or the genome of the subject, the lifestyle change is considered to be unsuccessful. In such cases, different and/or additional lifestyle changes can be recommended to the subject for achieving the desired result.

Exposure to mutagens, changes in environment or changes in lifestyle can affect the overall health and well-being of humans, including altering the chances of cancer development and/or changing the rate of aging. An embodiment of the invention provides a method of determining whether a lifestyle change or a combination of lifestyle changes, exposure to mutagens, or changes in environment altered the risk of cancer development of a subject and/or changed the rate of aging. For example, a person's risk of cancer development, such as a cancer of a particular tissue or organ, and/or the rate of aging as indicated by the genomic age can be determined before and after the lifestyle change was initiated. Non-limiting examples of lifestyle changes which can alter a subject's risk of cancer development and/or rate of aging include weight gain, smoking, exposure to a known carcinogen, change of a profession or job causing increased exposure to a particular carcinogen, dietary changes, etc. Additional examples of lifestyle changes that can alter the risk of cancer development and/or change the rate of aging are well-known to a person of ordinary skill in the art and such embodiments are within the purview of the invention.

Accordingly, an embodiment of the invention provides a method of identifying the effect of a lifestyle change on the risk of cancer development and/or the rate of aging of a subject. The method comprises:

a) according to the clamp/dPCR combination assay, determining the rate of mutations in a target sequence within a target SINE or target LINE and/or in the genome of the subject immediately before the lifestyle change is initiated,

b) according to the clamp/dPCR combination assay, determining the rate of mutations in the target sequence within the target SINE or target LINE and/or in the genome of the subject after the lifestyle change is initiated, and

c) comparing the rates of mutations in the target sequence within the target SINE or target LINE and/or the genome of the subject before and after the lifestyle change is initiated to determine the effect of the lifestyle change on the risk of cancer development and/or the rate of aging of the subject.

If the risk of cancer development increases and/or the rate of aging increases, indicated, for example, by a higher rate of mutations in a target sequence in a target SINE, target LINE and/or the genome of the subject, the lifestyle change is considered to increase the risk of cancer development and/or increase the rate of aging. The subject can then be recommended to either eliminate the lifestyle change which increased the risk of cancer and/or increased the rate of aging. Alternately, another lifestyle change intended to counter the earlier lifestyle change can be recommended.

On the other hand, if the risk of cancer development and/or the rate of aging does not increase or does not increase to an alarming extent, indicated, for example, by no increase or lack of increase to an alarming extent in the rate of mutations in a target sequence in a target SINE, target LINE and/or the genome of the subject, the lifestyle change is considered to be harmless. In such cases, no unnecessary changes in lifestyle are recommended.

Similarly, an embodiment of the invention provides a method of identifying the effect of an exposure to a mutagen on the risk of cancer development and/or the rate of aging of a subject. The method comprises:

a) according to the clamp/dPCR combination assay, determining the rate of mutations in a target sequence within a target SINE or target LINE and/or in the genome of the subject immediately before the exposure to the mutagen,

b) according to the clamp/dPCR combination assay, determining the rate of mutations in the target sequence within the target SINE or target LINE and/or in the genome of the subject after the exposure to the mutagen, and

c) comparing the rates of mutations in the target sequence within the target SINE or target LINE and/or the genome of the subject before and after the exposure to the mutagen to determine the effect of the exposure to the mutagen on the risk of cancer development and/or the rate of aging of the subject.

A further embodiment of the invention provides a kit comprising reagents to carry out the clamp/dPCR assay of the invention. In one embodiment, the kit comprises primers and/or probes specific for a SINE of interest in a species of interest. The kit can also comprise chemicals for treating the tissue or the genomic DNA sample obtained from the subject, for example, deproteination, degradation of non-DNA nucleotides, removal of other impurities, etc. The kit can further contain reagents and/or instrumentation for fractionating the genomic DNA into fragments of a desired size. Additionally, the kit can contain reagents and/or instrumentation for conducting the dPCR reaction. A manual containing instructions to carry out various methods of the invention can also be included in the kit.

TABLE 5
LINE1 sequences for humans (Homo sapiens)

1. Homo sapiens SLCO1B3 gene for solute carrier organic anion
transporter family, member 1B3, partial cds, exon 7 is excluded
due to the insertion of LINE1
7,685 bp linear DNA
AB896715.1 GI:1108831814
2. Homo sapiens partial LINE1 retrotransposon, clone HS3_6A
573 bp linear DNA
LT593639.1 GI:1127900357
3. Homo sapiens partial LINE1 retrotransposon, clone HS3_5H
574 bp linear DNA
LT593638.1 GI:1127900354
4. Homo sapiens partial LINE1 retrotransposon, clone HS3_5G
570 bp linear DNA
LT593637.1 GI:1127900351
5. Homo sapiens partial LINE1 retrotransposon, clone HS3_5F
566 bp linear DNA
LT593636.1 GI:1127900349
6. Homo sapiens partial LINE1 retrotransposon, clone HS3_5E
573 bp linear DNA
LT593635.1 GI:1127900342
7. Homo sapiens partial LINE1 retrotransposon, clone HS3_5D
570 bp linear DNA
LT593634.1 GI:1127900339
8. Homo sapiens partial LINE1 retrotransposon, clone HS3_5C
565 bp linear DNA
LT593633.1 GI:1127900335
9. Homo sapiens partial LINE1 retrotransposon, clone HS3_5B
573 bp linear DNA
LT593632.1 GI:1127900331
10. Homo sapiens partial LINE1 retrotransposon, clone HS3_5A
570 bp linear DNA
LT593631.1 GI:1127900327
11. Homo sapiens partial LINE1 retrotransposon, clone HS3_4G
567 bp linear DNA
LT593630.1 GI:1127900323
12. Homo sapiens partial LINE1 retrotransposon, clone HS3_4D
573 bp linear DNA
LT593629.1 GI:1127900320
13. Homo sapiens partial LINE1 retrotransposon, clone HS3_4C
576 bp linear DNA
LT593628.1 GI:1127900317
14. Homo sapiens partial LINE1 retrotransposon, clone HS3_4B
571 bp linear DNA
LT593627.1 GI:1127900313
15. Homo sapiens partial LINE1 retrotransposon, clone HS3_4A
573 bp linear DNA
LT593626.1 GI:1127900309
16. Homo sapiens partial LINE1 retrotransposon, clone HS3_3G
573 bp linear DNA
LT593625.1 GI:1127900304
17. Homo sapiens partial LINE1 retrotransposon, clone HS3_3F
573 bp linear DNA
LT593624.1 GI:1127900300
18. Homo sapiens partial LINE1 retrotransposon, clone HS3_3E
573 bp linear DNA
LT593623.1 GI:1127900296
19. Homo sapiens partial LINE1 retrotransposon, clone HS3_3D
573 bp linear DNA
LT593622.1 GI:1127900292
20. Homo sapiens partial LINE1 retrotransposon, clone HS3_3B
573 bp linear DNA
LT593621.1 GI:1127900288
21. Homo sapiens partial LINE1 retrotransposon, clone HS3_3A
572 bp linear DNA
LT593620.1 GI:1127900283
22. Homo sapiens partial LINE1 retrotransposon, clone HS3_2H
565 bp linear DNA
LT593619.1 GI:1127900278
23. Homo sapiens partial LINE1 retrotransposon, clone HS3_2G
579 bp linear DNA
LT593618.1 GI:1127900272
24. Homo sapiens partial LINE1 retrotransposon, clone HS3_2F
572 bp linear DNA
LT593617.1 GI:1127900268
25. Homo sapiens partial LINE1 retrotransposon, clone HS3_2E
573 bp linear DNA
LT593616.1 GI:1127900264
26. Homo sapiens partial LINE1 retrotransposon, clone HS3_2D
573 bp linear DNA
LT593615.1 GI:1127900259
27. Homo sapiens partial LINE1 retrotransposon, clone HS3_2C
575 bp linear DNA
LT593614.1 GI:1127900255
28. Homo sapiens partial LINE1 retrotransposon, clone HS3_2B
571 bp linear DNA
LT593613.1 GI:1127900250
29. Homo sapiens partial LINE1 retrotransposon, clone HS3_2A
573 bp linear DNA
LT593612.1 GI:1127900245
30. Homo sapiens partial LINE1 retrotransposon, clone HS3_1F
573 bp linear DNA
LT593611.1 GI:1127900241
31. Homo sapiens partial LINE1 retrotransposon, clone HS3_1E
572 bp linear DNA
LT593610.1 GI:1127900237
32. Homo sapiens partial LINE1 retrotransposon, clone HS3_1D
569 bp linear DNA
LT593609.1 GI:1127900234
33. Homo sapiens partial LINE1 retrotransposon, clone HS3_1B
573 bp linear DNA
LT593608.1 GI:1127900229
34. Homo sapiens partial LINE1 retrotransposon, clone HS3_1A
573 bp linear DNA
LT593607.1 GI:1127900225
35. Homo sapiens partial LINE1 retrotransposon, clone HS2_6H
572 bp linear DNA
LT593606.1 GI:1127900221
36. Homo sapiens partial LINE1 retrotransposon, clone HS2_6G
574 bp linear DNA
LT593605.1 GI:1127900217
37. Homo sapiens partial LINE1 retrotransposon, clone HS2_6F
573 bp linear DNA
LT593604.1 GI:1127900214
38. Homo sapiens partial LINE1 retrotransposon, clone HS2_6D
571 bp linear DNA
LT593603.1 GI:1127900210
39. Homo sapiens partial LINE1 retrotransposon, clone HS2_6A
564 bp linear DNA
LT593602.1 GI:1127900209
40. Homo sapiens partial LINE1 retrotransposon, clone HS2_5H
569 bp linear DNA
LT593601.1 GI:1127900206
41. Homo sapiens partial LINE1 retrotransposon, clone HS2_5G
567 bp linear DNA
LT593600.1 GI:1127900202
42. Homo sapiens partial LINE1 retrotransposon, clone HS2_5F
568 bp linear DNA
LT593599.1 GI:1127900199
43. Homo sapiens partial LINE1 retrotransposon, clone HS2_5D
572 bp linear DNA
LT593598.1 GI:1127900195
44. Homo sapiens partial LINE1 retrotransposon, clone HS2_5C
573 bp linear DNA
LT593597.1 GI:1127900191
45. Homo sapiens partial LINE1 retrotransposon, clone HS2_5B
573 bp linear DNA
LT593596.1 GI:1127900187
46. Homo sapiens partial LINE1 retrotransposon, clone HS2_5A
569 bp linear DNA
LT593595.1 GI:1127900183
47. Homo sapiens partial LINE1 retrotransposon, clone HS2_4G
573 bp linear DNA
LT593594.1 GI:1127900178
48. Homo sapiens partial LINE1 retrotransposon, clone HS2_4F
575 bp linear DNA
LT593593.1 GI:1127900173
49. Homo sapiens partial LINE1 retrotransposon, clone HS2_4E
573 bp linear DNA
LT593592.1 GI:1127900167
50. Homo sapiens partial LINE1 retrotransposon, clone HS2_4D
573 bp linear DNA
LT593591.1 GI:1127900162
51. Homo sapiens partial LINE1 retrotransposon, clone HS2_4C
568 bp linear DNA
LT593590.1 GI:1127900156
52. Homo sapiens partial LINE1 retrotransposon, clone HS2_4B
572 bp linear DNA
LT593589.1 GI:1127900153
53. Homo sapiens partial LINE1 retrotransposon, clone HS2_4A
572 bp linear DNA
LT593588.1 GI:1127900148
54. Homo sapiens partial LINE1 retrotransposon, clone HS2_3F
567 bp linear DNA
LT593587.1 GI:1127900146
55. Homo sapiens partial LINE1 retrotransposon, clone HS2_3D
573 bp linear DNA
LT593586.1 GI:1127900142
56. Homo sapiens partial LINE1 retrotransposon, clone HS2_3C
573 bp linear DNA
LT593585.1 GI:1127900139
57. Homo sapiens partial LINE1 retrotransposon, clone HS2_3B
561 bp linear DNA
LT593584.1 GI:1127900135
58. Homo sapiens partial LINE1 retrotransposon, clone HS2_3A
573 bp linear DNA
LT593583.1 GI:1127900132
59. Homo sapiens partial LINE1 retrotransposon, clone HS2_2G
573 bp linear DNA
LT593582.1 GI:1127900127
60. Homo sapiens partial LINE1 retrotransposon, clone HS2_2F
573 bp linear DNA
LT593581.1 GI:1127900123
61. Homo sapiens partial LINE1 retrotransposon, clone HS2_2E
573 bp linear DNA
LT593580.1 GI:1127900119
62. Homo sapiens partial LINE1 retrotransposon, clone HS2_2D
574 bp linear DNA
LT593579.1 GI:1127900115
63. Homo sapiens partial LINE1 retrotransposon, clone HS2_2C
573 bp linear DNA
LT593578.1 GI:1127900110
64. Homo sapiens partial LINE1 retrotransposon, clone HS2_2B
569 bp linear DNA
LT593577.1 GI:1127900108
65. Homo sapiens partial LINE1 retrotransposon, clone HS2_1H
573 bp linear DNA
LT593576.1 GI:1127900104
66. Homo sapiens partial LINE1 retrotransposon, clone HS2_1G
572 bp linear DNA
LT593575.1 GI:1127900101
67. Homo sapiens partial LINE1 retrotransposon, clone HS2_1F
574 bp linear DNA
LT593574.1 GI:1127900097
68. Homo sapiens partial LINE1 retrotransposon, clone HS2_1E
570 bp linear DNA
LT593573.1 GI:1127900095
69. Homo sapiens partial LINE1 retrotransposon, clone HS2_1D
569 bp linear DNA
LT593572.1 GI:1127900090
70. Homo sapiens partial LINE1 retrotransposon, clone HS2_1C
573 bp linear DNA
LT593571.1 GI:1127900087
71. Homo sapiens partial LINE1 retrotransposon, clone HS2_1B
573 bp linear DNA
LT593570.1 GI:1127900083
72. Homo sapiens partial LINE1 retrotransposon, clone HS1_5G
571 bp linear DNA
LT593569.1 GI:1127900079
73. Homo sapiens partial LINE1 retrotransposon, clone HS1_5D
573 bp linear DNA
LT593568.1 GI:1127900075
74. Homo sapiens partial LINE1 retrotransposon, clone HS1_5B
573 bp linear DNA
LT593567.1 GI:1127900072
75. Homo sapiens partial LINE1 retrotransposon, clone HS1_5A
573 bp linear DNA
LT593566.1 GI:1127900069
76. Homo sapiens partial LINE1 retrotransposon, clone HS1_4H
572 bp linear DNA
LT593565.1 GI:1127900066
77. Homo sapiens partial LINE1 retrotransposon, clone HS1_4G
573 bp linear DNA
LT593564.1 GI:1127900061
78. Homo sapiens partial LINE1 retrotransposon, clone HS1_4F
572 bp linear DNA
LT593563.1 GI:1127900057
79. Homo sapiens partial LINE1 retrotransposon, clone HS1_4E
599 bp linear DNA
LT593562.1 GI:1127900054
80. Homo sapiens partial LINE1 retrotransposon, clone HS1_4D
573 bp linear DNA
LT593561.1 GI:1127900051
81. Homo sapiens partial LINE1 retrotransposon, clone HS1_4C
568 bp linear DNA
LT593560.1 GI:1127900048
82. Homo sapiens partial LINE1 retrotransposon, clone HS1_4B
567 bp linear DNA
LT593559.1 GI:1127900044
83. Homo sapiens partial LINE1 retrotransposon, clone HS1_4A
573 bp linear DNA
LT593558.1 GI:1127900041
84. Homo sapiens partial LINE1 retrotransposon, clone HS1_3H
573 bp linear DNA
LT593557.1 GI:1127900037
85. Homo sapiens partial LINE1 retrotransposon, clone HS1_3G
569 bp linear DNA
LT593556.1 GI:1127900033
86. Homo sapiens partial LINE1 retrotransposon, clone HS1_3F
573 bp linear DNA
LT593555.1 GI:1127900029
87. Homo sapiens partial LINE1 retrotransposon, clone HS1_3D
573 bp linear DNA
LT593554.1 GI:1127900028
88. Homo sapiens partial LINE1 retrotransposon, clone HS1_2H
573 bp linear DNA
LT593553.1 GI:1127900024
89. Homo sapiens partial LINE1 retrotransposon, clone HS1_2G
569 bp linear DNA
LT593552.1 GI:1127900021
90. Homo sapiens partial LINE1 retrotransposon, clone HS1_2F
573 bp linear DNA
LT593551.1 GI:1127900017
91. Homo sapiens partial LINE1 retrotransposon, clone HS1_2D
573 bp linear DNA
LT593550.1 GI:1127900013
92. Homo sapiens partial LINE1 retrotransposon, clone HS1_2C
569 bp linear DNA
LT593549.1 GI:1127900009
93. Homo sapiens partial LINE1 retrotransposon, clone HS1_2B
570 bp linear DNA
LT593548.1 GI:1127900005
94. Homo sapiens partial LINE1 retrotransposon, clone HS1_2A
573 bp linear DNA
LT593547.1 GI:1127900002
95. Homo sapiens partial LINE1 retrotransposon, clone HS1_1H
573 bp linear DNA
LT593546.1 GI:1127899997
96. Homo sapiens partial LINE1 retrotransposon, clone HS1_1F
573 bp linear DNA
LT593545.1 GI:1127899992
97. Homo sapiens partial LINE1 retrotransposon, clone HS1_1E
573 bp linear DNA
LT593544.1 GI:1127899988
98. Homo sapiens partial LINE1 retrotransposon, clone HS1_1D
573 bp linear DNA
LT593543.1 GI:1127899984
99. Homo sapiens partial LINE1 retrotransposon, clone HS1_1C
573 bp linear DNA
LT593542.1 GI:1127899980
100. Homo sapiens partial LINE1 retrotransposon, clone HS1_1B
573 bp linear DNA
LT593541.1 GI:1127899975
101. Homo sapiens partial LINE1 retrotransposon, clone HS1_1A
573 bp linear DNA
LT593540.1 GI:1127899972
102. Homo sapiens LINE1 type transposase domain containing 1
(L1TD1), transcript variant 2, mRNA
3,894 bp linear mRNA
NM_019079.4 GI:258679512
103. Homo sapiens LINE1 type transposase domain containing 1
(L1TD1), transcript variant 1, mRNA
4,005 bp linear mRNA
NM_001164835.1 GI:258679510
104. Homo sapiens transcribed RNA, LINE1 antisense promoter
(L1ASP)-derived chimeric transcript, clone: LCT26-SP6
136 bp linear transcribed-RNA
AB755817.1 GI:457866444
105. Homo sapiens transcribed RNA, LINE1 antisense promoter
(L1ASP)-derived chimeric transcript, clone: LCT25-SP6
195 bp linear transcribed-RNA
AB755816.1 GI:457866443
106. Homo sapiens transcribed RNA, LINE1 antisense promoter
(L1ASP)-derived chimeric transcript, clone: LCT24-SP6
312 bp linear transcribed-RNA
AB755815.1 GI:457866442
107. Homo sapiens transcribed RNA, LINE1 antisense promoter
(L1ASP)-derived chimeric transcript, clone: LCT22-SP6
338 bp linear transcribed-RNA
AB755814.1 GI:457866441
108. Homo sapiens LINE1 element inserted in B-globin gene intron 2
6,045 bp linear DNA
AF149422.1 GI:5052949
109. Human LINE1 (L1.4) repetitive element DNA sequence
6,053 bp linear DNA
L19092.1 GI:307102
110. Human LINE1 (L1.4) repetitive element DNA sequence
351 bp linear DNA
L19091.1 GI:307101
111. Human LINE1 (L1.4) repetitive element DNA sequence
279 bp linear DNA
L19090.1 GI:307100
112. Human LINE1 (L1.4) repetitive element DNA sequence
389 bp linear DNA
L19089.1 GI:307099
113. Human LINE1 (L1.3) repetitive element DNA sequence
6,059 bp linear DNA
L19088.1 GI:307098
114. Human LINE1 (L1.3) repetitive element DNA sequence
139 bp linear DNA
L19087.1 GI:307097
115. Human LINE1 (L1.3) repetitive element DNA sequence
541 bp linear DNA
L19086.1 GI:307096
116. Homo sapiens 5′ flanking region of LINE1 element within
the apo(a)-plasminogen intergenic region
543 bp linear DNA
AJ316226.2 GI:40644101
117. Homo sapiens fragile X mental retardation syndrome
protein (FMR1) gene, alternative splice products, complete cds;
and pseudogene, complete sequence
185,775 bp linear DNA
L29074.1 GI:1668818
118. Homo sapiens cosmid HGAB from chromosome 13,
complete sequence
36,188 bp linear DNA
AC002982.1 GI:2443900
119. Homo sapiens germline beta T-cell receptor locus
684,973 bp linear DNA
L36092.2 GI:38492353
120. Human cosmid insert containing polymorphic marker DXS455
38,409 bp linear DNA
L31948.1 GI:473756
121. Homo sapiens chromosome 4 Morf4 protein (MORF4) gene,
complete cds
4,370 bp linear DNA
AF100614.2 GI:6960302
122. Homo sapiens chromosome 15 clone RP11-947O24 map 15q21.1,
complete sequence
188,439 bp linear DNA
AC025919.8 GI:19071579
123. Homo sapiens serine/threonine kinase 32B (STK32B),
RefSeqGene on chromosome 4
456,679 bp linear DNA
NG_051593.1 GI:1067605104
124. Homo sapiens serine/threonine kinase 32B (STK32B),
transcript variant 3, mRNA
3,420 bp linear mRNA
NM_001345969.1 GI:1066566378
125. Homo sapiens serine/threonine kinase 32B (STK32B),
transcript variant 2, mRNA
3,639 bp linear mRNA
NM_001306082.1 GI:807045872
126. Homo sapiens serine/threonine kinase 32B (STK32B),
transcript variant 1, mRNA
3,529 bp linear mRNA
NM_018401.2 GI:807045869
127. Homo sapiens long intergenic non-protein coding RNA 1587
(LINC01587), transcript variant 2, long non-coding RNA
888 bp linear ncRNA, lncRNA
NR_126518.1 GI:723802116
128. Homo sapiens long intergenic non-protein coding RNA 1587
(LINC01587), transcript variant 3, long non-coding RNA
797 bp linear ncRNA, lncRNA
NR_126519.1 GI:723802114
129. Homo sapiens long intergenic non-protein coding RNA 1587
(LINC01587), transcript variant 1, long non-coding RNA
909 bp linear ncRNA, lncRNA
NR_126517.1 GI:723802112
130. Homo sapiens insertion sequence TMF1, complete sequence
5,839 bp linear DNA
KJ027511.1 GI:572609680
131. Homo sapiens DNA, replication enhancing element (REE1)
10,199 bp linear DNA
D50561.1 GI:1167504
132. Homo sapiens transposon L1PMA2 5′ LINE1-like repetitive
element
795 bp linear DNA
AJ426051.1 GI:22035757
133. Homo sapiens retrotransposon L1 insertion in X-linked
retinitis pigmentosa locus, complete sequence
6,019 bp linear DNA
AF148856.1 GI:5070620
134. Human chromosome X cosmid, clones 196B12, 9H11 and 43H9,
repeat units and sequence tagged sites
106,000 bp linear DNA
U40455.1 GI:1079752
135. Homo sapiens pre-integration site of LINE1-mediated
deletion LH28 genomic sequence
876 bp linear DNA
DQ017971.1 GI:67848469
136. Homo sapiens pre-integration site of LINE1-mediated
deletion LH26 genomic sequence
448 bp linear DNA
DQ017970.1 GI:67848468
137. Homo sapiens pre-integration site of LINE1-mediated
deletion LH24 genomic sequence
700 bp linear DNA
DQ017969.1 GI:67848467
138 Homo sapiens pre-integration site of LINE1-mediated
deletion LH19 genomic sequence
504 bp linear DNA
DQ017968.1 GI:67848466
139. Homo sapiens pre-integration site of LINE1-mediated
deletion LH15 genomic sequence
199 bp linear DNA
DQ017967.1 GI:67848465
140. Homo sapiens tyrosinase related protein 1 (TYRP1)
gene, complete cds
24,667 bp linear DNA
AF001295.1 GI:2735661
141. Homo sapiens son-pseudogene
7,290 bp linear DNA
X71604.1 GI:296950
142. Homo sapiens partial gene for novel KRAB protein domain,
exons 1-2
1,488 bp linear DNA
AJ245586.1 GI:5730193
143. H. sapiens sequence involved in X;Y translocation
2,563 bp linear DNA
X70412.1 GI:515818
144. H. sapiens gene for immunoglobulin kappa light chain
variable region A6
2,834 bp linear DNA
X71886.1 GI:434965
145. Homo sapiens clone CC36281C1C4C7 NADP-dependent retinol
dehydrogenase/reductase (DHRS4) gene, exons 6.2 through 8.1
3,087 bp linear DNA
DQ149231.1 GI:73918039
146. Homo sapiens clone CA16281C4 NADP-dependent retinol
dehydrogenase/reductase (DHRS4) gene, exons 6.2 through 8.1
3,087 bp linear DNA
DQ149227.1 GI:73918035
147. Homo sapiens clone CA16282C8 NADP-dependent retinol
dehydrogenase/reductase (DHRS4) gene, exons 6.2 through 8.2
3,083 bp linear DNA
DQ149225.1 GI:73918033
148. Homo sapiens Yp11.2/Xp22.3 junction fragment in
46, X, t(X;Y) patient lhda
2,874 bp linear DNA
AJ309278.1 GI:14270371
149. Homo sapiens STS BAC clone L0974, SP6 end, sequence
tagged site
703 bp linear DNA
AJ250574.1 GI:6688215
150. Homo sapiens clone LS535G M3 hypoxanthine
phosphoribosyltransferase (hprt) 50 kb deletion mutant mRNA,
partial cds, containing human LINE element
471 bp linear mRNA
U31732.1 GI:940938
151. H. sapiens sequence involved in X;Y translocation
2,469 bp linear DNA
X70413.1 GI:515034

TABLE 6
LINE1 sequences for mice (Mus musculus)

1. Mus musculus clone ma15 L1 retrotransposon LINE1 repeat region
204 bp linear DNA
U80266.1 GI:1737245
2. Mus musculus clone ma14 L1 retrotransposon LINE1 repeat region
207 bp linear DNA
U80265.1 GI:1737244
3. Mus musculus clone ma13 L1 retrotransposon LINE1 repeat region
187 bp linear DNA
U80264.1 GI:1737243
4. Mus musculus clone ma12 L1 retrotransposon LINE1 repeat region
201 bp linear DNA
U80263.1 GI:1737242
5. Mus musculus clone ma11 L1 retrotransposon LINE1 repeat region
197 bp linear DNA
U80262.1 GI:1737241
6. Mus musculus clone ma10 L1 retrotransposon LINE1 repeat region
201 bp linear DNA
U80261.1 GI:1737240
7. Mus musculus clone ma9 L1 retrotransposon LINE1 repeat region
206 bp linear DNA
U80260.1 GI:1737239
8. Mus musculus clone ma8 L1 retrotransposon LINE1 repeat region
205 bp linear DNA
U80259.1 GI:1737238
9. Mus musculus clone ma7 L1 retrotransposon LINE1 repeat region
205 bp linear DNA
U80258.1 GI:1737237
10. Mus musculus clone ma6 L1 retrotransposon LINE1 repeat region
206 bp linear DNA
U80257.1 GI:1737236
11. Mus musculus clone ma5 L1 retrotransposon LINE1 repeat region
206 bp linear DNA
U80256.1 GI:1737235
12. Mus musculus clone ma4 L1 retrotransposon LINE1 repeat region
205 bp linear DNA
U80255.1 GI:1737234
13. Mus musculus clone ma3 L1 retrotransposon LINE1 repeat region
199 bp linear DNA
U80254.1 GI:1737233
14. Mus musculus clone ma2 L1 retrotransposon LINE1 repeat region
204 bp linear DNA
U80253.1 GI:1737232
15. Mus musculus clone ma1 L1 retrotransposon LINE1 repeat region
194 bp linear DNA
U80252.1 GI:1737231
16. Mus musculus LINE1 repeat DNA
324 bp linear DNA
X58284.1 GI:52937
17. Mus musculus proteasome activator PA28 beta subunit (PSME2b)
gene, complete cds
2,261 bp linear DNA
AF 115502.1 GI:5031227
18. Mus musculus protease (prosome, macropain) activator subunit 2B
(Psme2b), mRNA
1,066 bp linear mRNA
NM_001281472.1 GI:527317389
19. Mus musculus hypothetical protein RDA63 gene, complete cds
19,216 bp linear DNA
AF442737.1 GI:17298528
20. Mus musculus strain 129X1/SvJ tyrosinase locus control region
13,806 bp linear DNA
AF364302.1 GI:22506679
21. Mus musculus piwi-like RNA-mediated gene silencing 2 (Piwil2),
mRNA 4,913 bp linear mRNA
NM_021308.1 GI:10946609
22. Mus musculus tudor domain containing 1 (Tdrd1), transcript variant 3,
mRNA 5,187 bp linear mRNA
NM_001002241.2 GI:268607548
23. Mus musculus tudor domain containing 1 (Tdrd1), transcript variant 2,
mRNA 4,887 bp linear mRNA
NM_001002240.2 GI:268607546
24. Mus musculus tudor domain containing 1 (Tdrd1), transcript variant 1,
mRNA 4,914 bp linear mRNA
NM_001002238.2 GI:268607545
25. Mus musculus tudor domain containing 1 (Tdrd1), transcript variant 4,
mRNA 5,047 bp linear mRNA
NM_031387.3 GI:268607543
26. Mus musculus tudor domain containing 9 (Tdrd9), mRNA
4,809 bp linear mRNA
NM_029056.1 GI: 198278550
27. Mus musculus piwi-like RNA-mediated gene silencing 4 (Piwil4),
mRNA 2,637 bp linear mRNA
NM_177905.3 GI:52138555
28. Mus musculus Apexpl, Cbx3p1 psudogenes for Apex nuclease,
chromobox homolog 3
14,773 bp linear DNA
AB084238.2 GI:51699492
29. Mus musculus DNA, clone:pINS_hurl 70, insertion mutant
2,747 bp linear DNA
AB104439.1 GI:28569959
30. Mus musculus CC chemokine LEC pseudogene exons
5,728 bp linear DNA
AB018250.1 GI:4033628
31. Mus musculus ALDR gene including 5′UTR and promoter, partial
3,812 bp linear DNA
AJ009992.2 GI:7209181
32. Mus musculus beige gene, LINE1 repetitive element
2,068 bp linear DNA
U78038.1 GI:2209022
33. M.musculus SPRR3 gene
2,474 bp linear DNA
Y09227.1 GI:3157400

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—CLAMP/DPCR of ALU, a Human SINE and B1, a Mouse SINE

Protein-coding regions of the human genome occupy only ˜1.5% of the DNA, accounting for approximately 21,000 genes on the 23 chromosomes. A large component of the remaining DNA is composed of SINEs. Alu elements are the most abundant SINE in the human genome. Similarly, B1 elements are the most abundant SINE in the mouse genome. Alu elements are short with approximately 300-350 base pairs and contain a restriction enzyme site. With approximately 500,000 to 1,500,000 copies, B1 elements and Alu make up about 11% of the mouse and human genomes, respectively.

An embodiment of the invention provides assaying point mutations in 11% of the genome formed by the Alu elements. The rate of mutations in the genome-wide Alu elements can be used to obtain an accurate estimation of mutations in the genome.

Accordingly, in one embodiment, the invention provides a clamp/dPCR assay for Alu, a human SINE, to serially quantify the total Alu mutations in an organ in a subject, for example, examining 10⁹ Alu loci in an hour. In another embodiment, the invention provides a method of determining the accumulated mutations in Alu. In an even further embodiment, the invention provides a method of using the accumulated mutations in conjunction with chronological age and Surveillance, Epidemiology and End Results (SEER) cancer statistics to quantitatively predict cancer risk (FIG. 1).

The clamp/dPCR assay of the invention can detect 1-2 mutant DNA fragments in a pool of 100,000 wild-type fragments (FIG. 6), and quantitatively differentiate 1-2 mutations from 5-10 mutations in a pool of 100,000 wild-type fragments. An appropriately chosen target SINE, for example, a SINE sequence that is about 10% prevalent in the genomic DNA and is about 300-400 bp, assures that in a mixture of genomic DNA fragments more than 10% will contain the target SINE. Accordingly, the prevalence and short lengths of Alu and B1 assure that in a mix of genomic DNA fragments from a human and a mouse, respectively, more than 10% will contain the sequence.

In a 45-min cycle of a dPCR, for example, ddPCR (BioRad QX200 AutoDG ddPCR, Hercules, Calif.), over 10 DNA fragments can be analyzed in each of the 8 channels for the presence of mutations. The clamp/dPCR assay of the invention allows using 100 or 1,000 fragments per drop instead of ˜1-2 fragments per drop because the target sequence clamp prevents amplification in most of the target SINE sequences that are likely to be wild-type. Since about 10% of those DNA fragments likely contain the target SINE, ˜10⁵ target SINEs are analyzed at one fragment per well or drop. Therefore, the clamp/dPCR combination assay of the invention improves the screening of Alu fragments by 2 or 3 orders of magnitude, i.e., up to 10⁸ Alu/channel. Assuming 10⁻⁶ mutations per cell division and/or per week of age, the assay has the capacity to estimate the rate of mutations in genome-wide Alu sequences.

Target sequence clamps for mouse B1 and human Alu with base sizes of 16-20 are provided. In a test run, excess idealized wild-type B1 with a trace of mutant B was tested using a 16-base Clamp2 (SEQ ID NO: 4) at the dilutions of 1:1000 and 1:10,000. Additional clamps can be designed, and alleles for any clamp can be prepared and multiplexed to eliminate common variations in SINE sequences.

According to an embodiment, any tissue can be tested with the clamp/dPCR combination assay. For example, in humans, a skin or mucosal scrape or 1-2 μl of blood is sufficient. Since reagents used in routine PCR are used, the assay provides high quality and reproducibility. Therefore, an assay can be repeated economically in the same subject or organ.

### Example 2—Estimation of Genome-Wide Mutations after Particle Irradiation in Various Tissues

Like natural age-related genome-wide accumulated mutations and/or rate of mutations, radiation-related genome-wide accumulated mutations and/or rate of mutations occur at different rates in individuals and directly lead to different risks and rates for cancer. Radiation-induced mutations should be random with respect to their distribution across the genome. Although some location-specific effects likely arise due to repair mechanisms, testing for mutations near specific genes is likely to be futile. On the other hand, overall mutation load, e.g., the genome-wide accumulated mutations and/or the rate of mutations and the incidence of driver mutations have a linear relationship.

A dose- and LET-dependent increase in genome-wide mutations is expected. Basal and serial studies can be performed following ¹H, ¹n, ²⁸Si, or ⁵⁶Fe irradiation (dose ≤0.5 Gy). Both sexes and individual organs can be studied. Several assays can be designed based on Table 7.

TABLE 7
Assay designs

| Hypothesis | Assay design |
|---|---|
| Age, radiation dose, and | Clamp/dPCR combination assay for B1 |
| LET affect the genome-wide | sequence in mice and Alu sequence in |
| mutations. | humans after particle radiation with, |
| | for example, ¹n, ¹H, ²⁸Si, and ⁵⁶Fe. |
| Genome-wide mutations in | Clamp/dPCR combination assay for B1 |
| spontaneous tumors can be | in mice with spontaneous tumors. |
| similar or higher than the | Tumor and normal tissue can be |
| genome-wide mutations in | examined from the same subject and |
| normal tissues. | same organ. |

Point and indel mutations increase with aging and radiation of different qualities and doses affect the rate of mutations. Point and indel mutation comprises over 90-95% of all mutations. The test provided in this example is simple and inexpensive and requires only 1-2 ng of DNA. As such, it can be performed on a number of mouse strains, on each individual animal and in different organs. Strains representing a range of cancer predilections (including sex-related cancers, such as breast and ovary) can also be studied. A preliminary evaluation of the various potential clamp sites can be made to determine the most robust set of target sequence clamps for use in an animal of interest, for example, a mouse strain.

Various tissues, including blood, muscle, brain, heart, lung, skin, breast, large bowel, liver, and spleen, can be tested for the effect of radiation. Because cancer rates increase in progeny after high LET, testes and ovarian tissues can be tested to evaluate germline genome-wide mutation levels. These organs are chosen because these organs are all known to have cancer predispositions following radiation exposure or, like skin, might sustain the highest radiation exposure.

Non-limiting examples of specific organs which can be tested according to this example of the invention include skin, lung, breast, and WBC. Skin receives a higher exposure than most organs and leukemia is common after irradiation. Also, WBC genome-wide mutations are needed for human comparison and lung and breast tumors are relatively common in mice and humans. These tissues are particularly preferred to study the effects of radiation.

The test can be carried on tissues obtained at 0 hours, 24 hours, 1 month, 6 months, 1 year, 2 to 5 years or longer after exposure to radiation. These analyses can be done on individual mice for animal-specific organ comparisons. Thus, intra- and inter-strain comparisons to compute the difference between the genomic age and the chronological age (Δ_{age}) can be calculated.

### Example 3—Measurements of Genome-Wide Mutations in Spontaneous Tumors and Normal Tissue

The number of the genome-wide mutations in a spontaneous tumor is similar to or larger than the number of these mutations in the normal tissue. A spontaneous tumor refers to a tumor which arises in a subject that is not exposed to known carcinogens or tumor-promoting factors, e.g., ionizing radiation, mutagens, oncogenic viruses, etc.

Tumors and the source tissue can be examined from the same subject. NIH Swiss white mice with a female to male ratio of 1:2 can be used. Having fewer females is also logical as breast and ovarian cancers are common in this strain, leading to good representation of females in the final tumor population. This strain has a ˜10-20% cumulative lifetime risk of malignancy, with lung>ovary>breast>leukemia>sarcoma>gastrointestinal (GI) cancers. The GI cancers include an even mix of stomach, colon and liver.

Genetically defined animals with cancer predilection can also be used. For example, the Kras^{JA1} model of lung cancer, the Apc heterozygote knockout model for GI cancers, as well as other cancer-predisposed models featuring Trp53^{-/-} can be used. However, as most of these mice develop cancer without radiation and simply demonstrate shorter latency or more aggressive pathology when irradiated, they are good models for radiation-induced cancer progression but less effective for emulating spontaneous carcinogenesis. Therefore, the strain chosen for studying passenger DNA damage (PDD) in spontaneous tumors should be a typically healthy strain.

Spontaneous oncogenesis studies can be long-term, with latency to cancer of 300-800 days in mice, and can involve large and laborious animal cohorts as the lifetime risks are only 10-20% in non-irradiated and 15-30% in irradiated animals. Certain algorithms can be used which do not require validation by correlation with the rate of cancers in animals, thereby allowing the use of smaller cohorts. Also, algorithms can be used which require the accumulation of DNA damage after irradiation to be allometrically scalable between species so that genomic age and Δ_{age} can be calculated. Accordingly, human epidemiological statistics can be applied to predict driver frequency and human cancer risk.

### Example 4—Measurement of PDD in Normal Human Aging

PDD mutations increase with normal human aging processes and can progress in different subjects at different rates. Subjects in the various age groups/ranges can be studied. PDD mutations are expected to correlate with increasing age.

Framework of PDD and Cancer Risk:

Subject-specific rates of genomic aging as markers for cancer risk can be calculated. Genomic aging can be quantified through serial measurements of point and indel mutations. A subject's genomic aging status can be tracked against age-related cancer incidence trajectories at the population level for risk estimation. The rate of PDD accumulation is a primary quantity of interest. Germline variations can be implicitly adjusted to confound the risk estimates. Specifically, at birth, there exist some number of germline abnormalities (point and indels) in each cell in the body. Notably, the overwhelming majority of germline abnormalities behave as passengers and do not confer heightened cancer risk. A subject's neonatal blood can be used as the subject's mutation-free state, which can be used to fully disentangle PDD accumulation over the subject's lifetime from benign germline variations. However, sequential measurements of PDD over a period can be used to estimate rates of future damage accumulation that can be compared against population averages to determine relative risk for a subject.

Established age-specific cancer incidence data which show that most cancers follow a power law in chronological age can be used as a frame of reference. In particular, incidence curves are commonly observed as I(t)∝t^{k}, where t and I(t) respectively denote chronological age and age-specific incidence. The constant k is estimated from population data and is specific to cancer type, sex, race, and other epidemiologic factors. Appropriate functional forms for population subgroups can be determined using the SEER data.

The occurrence of PDD is proportional to the occurrence of driver mutations and, hence, proportional to cancer incidence. Therefore,

$$\frac{d P D D}{d t} = c {t}^{k - 1} ,$$

where c is a constant (FIG. 7). Conveniently, the parameter of interest, c, may be estimated from sequential measurements for each subject, since

$$\frac{dPDD}{d t}$$

can be calculated as a simple difference, and k and t have been derived from the population analysis. This analysis does not require an assumption-laden back-calculation to cellular abnormalities at birth. In short, subjects with large estimated values of c will be identified as having a high genomic age and an elevated future risk of cancer, since their high mutation accumulation rate implies a history of harmful exposure and an enhanced likelihood of driver mutations.

Genomic aging can predict adverse responses to sustained low-dose irradiation. Flexible regression strategies (e.g., spline fitting) will be used to optimally link molecular markers of radiation sensitivity to PDD. Data from the non-irradiated animals can be paired with analogous data from human subjects to permit allometric scaling of marker quantities from mouse to human. The rate of PDD accumulation at age t and also PDD itself at age t through the system of equations specified separately for chronological age can be estimated. PDD is expected to represent a more comprehensive measure of accumulated DNA damage.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

1. Bozic I, Antal T, Ohtsuki H, Carter H, Kim D, Chen S, Karchin R, Kinzler K W, Vogelstein B, Nowak M A. Accumulation of driver and passenger mutations during tumor progression. Proc Natl Acad Sci USA. 2010; 107(43):18545-50.
2. Tomasetti C, Marchionni L, Nowak M A, Parmigiani G, Vogelstein B. Only three driver gene mutations are required for the development of lung and colorectal cancers. Proc Natl Acad Sci USA. 2015; 112(1):118-23.
3. Vogelstein B, Papadopoulos N, Velculescu V E, Zhou S, Diaz L A, Jr., Kinzler K W. Cancer genome landscapes. Science. 2013; 339(6127):1546-58.
4. Surveillance, Epidemiology, and End Results Program. Citations for SEER Databases and SEER*State Software. 2015. Available from: http://seer.cancer.gov/data/citation.html.
5. Teschendorff A E, West J, Beck S. Age-associated epigenetic drift: implications, and a case of epigenetic thrift? Hum Mol Genet. 2013; 22(R1):R7-R15.
6. Rakyan V K, Down T A, Maslau S, Andrew T, Yang T P, Beyan H, Whittaker P, McCann O T, Finer S, Valdes A M, Leslie R D, Deloukas P, Spector T D. Human aging-associated DNA hypermethylation occurs preferentially at bivalent chromatin domains. Genome Res. 2010; 20(4):434-9.
7. Cordaux R, Batzer M A. The impact of retrotransposons on human genome evolution. Natl Rev Genet. 2009; 10(10):691-703.
8. Chen J C, Alvarez M J, Talos F, Dhruv H, Rieckhof G E, lyer A, Diefes K L, Aldape K, Berens M, Shen M M, Califano A. Identification of causal genetic drivers of human disease through systems-level analysis of regulatory networks. Cell. 2014; 159(2):402-14.
9. Vandin F, Upfal E, Raphael B J. Finding driver pathways in cancer: models and algorithms. Algorithms Mol Biol. 2012; 7(1):23.
10. Rodriguez-Brenes I A, Komarova N L, Wodarz D. Evolutionary dynamics of feedback escape and the development of stem-cell-driven cancers. Proc Natl Acad Sci USA. 2011; 108(47):18983-8.
11. Rithidech K N, Honikel L, Whorton E B. mFISH analysis of chromosomal damage in bone marrow cells collected from CBA/CaJ mice following whole body exposure to heavy ions (56Fe ions). Radiat Environ Biophys. 2007; 46(2):137-45.
12. Tucker J D, Marples B, Ramsey M J, Lutze-Mann L H. Persistence of chromosome aberrations in mice acutely exposed to 56Fe+26 ions. Radiat Res 2004161(6):648-55.
13. Little M P, Kwon D, Doi K, Simon S L, Preston D L, Doody M M, Lee T, Miller J S, Kampa D M, Bhatti P, Tucker J D, Linet M S, Sigurdson A J. Association of chromosome translocation rate with low dose occupational radiation exposures in U.S. radiologic technologists. Radiat Res. 2014; 182(1):1-17.
14. Vorobtsova I E, Tucker J D, Timofeeva N M, Bogomazova A N, Semenov A V, Pleshanov P G. [Effect of age and radiation exposure on the frequency of translocations and dicentrics detected by FISH method in human lymphocytes]. Radiat Biol. 2000; 40(2):142-8.
15. Lindholm C, Makelainen I, Paile W, Koivistoinen A, Salomaa S. Domestic radon exposure and the frequency of stable or unstable chromosomal aberrations in lymphocytes. Int J Radiat Biol. 1999; 75(8):921-8.
16. Bell J T, Tsai P C, Yang T P, Pidsley R, Nisbet J, Glass D, Mangino M, Zhai G, Zhang F, Valdes A, Shin S Y, Dempster E L, Murray R M, Grundberg E. Hedman A K, Nica A, Small K S, Dermitzakis E T, McCarthy M I, Mill J, Spector T D, Deloukas P. Epigenome-wide scans identify differentially methylated regions for age and age-related phenotypes in a healthy ageing population. PLoS Genet. 2012; 8(4):e1002629.
17. Bollati V, Schwartz J, Wright R, Litonjua A, Tarantini L, Suh H, Sparrow D, Vokonas P, Baccarelli A. Decline in genomic DNA methylation through aging in a cohort of elderly subjects. Mech Ageing Dev. 2009; 130(4):234-9.
18. Christensen B C, Houseman E A, Marsit C J, Zheng S, Wrensch M R, Wiemels J L, Nelson H H, Karagas M R, Padbury J F, Bueno R, Sugarbaker D J, Yeh R F, Wiencke J K, Kelsey K T. Aging and environmental exposures alter tissue-specific DNA methylation dependent upon CpG island context. PLoS Genet. 2009; 5(8):e1000602.
19. Fraga M F, Esteller M. Epigenetics and aging: the targets and the marks. Trends Genet. 2007; 23(8):413-8.
20. Rodriguez-Rodero S, Fernandez-Morera J L, Fernandez A F, Menendez-Torre E, Fraga M F. Epigenetic regulation of aging. Discov Med. 2010; 10(52):225-33.
21. Mathers J C, Stathdee G, Relton C L. Induction of epigenetic alterations by dietary and other environmental factors. Adv Genet. 2010; 71:3-39.
22. Bell J T, Spector T D. DNA methylation studies using twins: what are they telling us? Genome Biol. 2012; 13(10):172.
23. Kilpinen H, Dermitzakis E T. Genetic and epigenetic contribution to complex traits. Hum Mol Genet. 2012; 21(R1):R24-8.
24. Leonhardt H, Page A W, Weier H U, Bestor T H. A targeting sequence directs DNA methyltransferase to sites of DNA replication in mammalian nuclei. Cell. 1992; 71(5):865-73.
25. Hannum G, Guinney J, Zhao L, Zhang L, Hughes G, Sadda S, Klotzle B, Bibikova M, Fan J B, Gao Y, Deconde R, Chen M, Rajapakse I, Friend S, Ideker T, Zhang K. Genome-wide methylation profiles reveal quantitative views of human aging rates. Mol Cell. 2013; 49(2):359-67.
26. Horvath S. DNA methylation age of human tissues and cell types. Genome Biol. 2013; 14(10):R115.
27. Marioni R E, Shah S, McRae A F, Chen B H, Colicino E, Harris S E, Gibson J, Henders A K, Redmond P, Cox S R, Pattie A, Corley J, Murphy L, Martin N G, Montgomery G W, Feinberg A P, Fallin M D, Multhaup M L, Jaffe A E, Joehanes R, Schwartz J, Just A C, Lunetta K L, Murabito J M, Starr J M, Horvath S, Baccarelli A A, Levy D, Visscher P M, Wray N R, Deary I J. DNA methylation age of blood predicts all-cause mortality in later life. Genome Biol. 2015; 16:25.
28. West J, Beck S, Wang X, Teschendorff A E. An integrative network algorithm identifies age-associated differential methylation interactome hotspots targeting stem-cell differentiation pathways. Sci Rep. 2013; 3:1630.
29. Maegawa S, Hinkal G, Kim H S, Shen L, Zhang L, Zhang J, Zhang N, Liang S, Donehower L A, Issa J P. Widespread and tissue specific age-related DNA methylation changes in mice. Genome Res. 2010; 20(3):332-40.
30. Teschendorff A E, Menon U, Gentry-Maharaj A, Ramus S J, Weisenberger D J, Shen H, Campan M, Noushmehr H, Bell C G, Maxwell A P, Savage D A, Mueller-Holzner E, Marth C, Kocjan G, Gayther S A, Jones A, Beck S Wagner W, Laird P W, Jacobs I J, Widschwendter M. Age-dependent DNA methylation of genes that are suppressed in stem cells is a hallmark of cancer. Genome Res. 2010; 20(4):440-6.
31. Horvath S, Zhang Y, Langfelder P, Kahn R S, Boks M P, van Eijk K, van den Berg L H, Ophoff R A. Aging effects on DNA methylation modules in human brain and blood tissue. Genome Biol. 2012; 13(10):R97.
32. Aubert G, Lansdorp P M. Telomeres and aging. Physiol Rev. 2008; 88(2):557-79.
33. Meissner C, Ritz-Timme S. Molecular pathology and age estimation. Forensic Sci Int. 2010; 203(1-3):34-43.
34. Zubakov D, Liu F, van Zelm M C, Venmeulen J, Oostra B A, van Duijn C M, Driessen G J, van Dongen J J, Kayser M, Langerak A W. Estimating human age from T-cell DNA rearrangements. Curr Boil. 2010; 20(22):R970-1.
35. Lin J, Epel E, Blackburn E. Telomeres and lifestyle factors: roles in cellular aging. Mutat Res. 2012; 730(1-2):85-9.
36. Pardo C E, Nabilsi N H, Darst R P, Kladde M P. Integrated DNA methylation and chromatin structural analysis at single-molecule resolution. Methods Mol Biol. 2015; 1288:123-41.
37. Nabilsi N H, Deleyrolle L P, Darst R P, Riva A, Reynolds B A, Kladde M P. Multiplex mapping of chromatin accessibility and DNA methylation within targeted single molecules identifies epigenetic heterogeneity in neural stem cells and glioblastoma. Genome Res. 2014; 24(2):329-39.
38. Chen Y, Hyrien O, Williams J, Okunieff P Smudzin T, Rubin P. Interleukin (IL)-1A and IL-6: applications to the predictive diagnostic testing of radiation pneumonitis. Int J Radiat Oncol Biol Phys. 2005; 62(0):260-6.
39. Rubin P, Johnston C J, Williams J P, McDonald S, Finkelstein J N. A perpetual cascade of cytokines postirradiation leads to pulmonary fibrosis. Int J Radiat Oncol Biol Phys. 1995; 33(1):99-109.
40. Xiao Z, Yang S, Su Y, Wang W, Zhang H, Zhang M, Zhang K, Tian Y, Cao Y, Yin L, Zhang L, Okunieff P. Alteration of the inflammatory molecule network after irradiation of soft tissue. Adv Exp Med Biol. 2013; 765:335-41.
41. Zhang M, Yin L, Zhang K, Sun W, Yang S, Zhang B, Salzman P, Wang W, Liu C, Vidyasagar S. Zhang L, Ju S, Okunieff P. Response patterns of cytokines/chemokines in two murine strains after irradiation. Cyokine. 2012; 58(2):169-77.
42. Liu W, Ding I, Chen K, Olschowka J, Xu J, Hu D, Morrow G R, Okunieff P. Interleukin 1beta (IL1B) signaling is a critical component of radiation-induced skin fibrosis. Radiat Res. 2006; 165(2):181-91.
43. Zhang S B, Okunieff P. Mitochondrial genetic abnormalities after radiation exposure. Adv Exp Med Biol. 2014; 812:1-7.
44. Zhang S B, Maguire D, Zhang M, Tian Y, Yang S Zhang A, Casey-Sawicki K, Han D, Ma J, Yin L, Guo Y, Wang X, Chen C, Litvinchuk A, Zhang Z, Swarts S, Vidyasagar S, Zhang L, Okunieff P. Mitochondrial DNA and functional investigations into the radiosensitivity of four mouse strains. Int J Cell Biol. 2014:2014:850460.
45. Zhang S B, Maguire D, Zhang M, Zhang Z, Zhang A, Yin L, Zhang L, Huang L, Vidyasagar S. Swarts S, Okunieff P. The murine common deletion: mitochondrial DNA 3,860-bp deletion after irradiation. Radiat Res. 2013; 180(4):407-13.
46. Zhang S B, Maguire D, Zhang M, Zhang A, Zhang L, Swarts S, Okunieff P. Maternal bias in mouse radiosensitivity: the role of the mitochondrial PTP. Adv Exp Med Biol. 2013; 789:251-6.
47. Zhang S B, Zhang M, Cao Y, Yang S, Zhang A P, Yin L, Tian Y, Zhang K, Guo Y, Wang X, Chen C, Ma J, Han D, Litvinchuk A, Swarts S, Vidyasagar S, Maguire D, Zhang L, Okunieff P. Delayed effects of radiation on mitochondrial DNA in radiation-sensitive organs. Adv Exp Med Biol. 2012; 737:139-45.
48. Scanlon S E, Glazer P M. Multifaceted control of DNA repair pathways by the hypoxic tumor microenvironment. DNA Repair (Amst). 2015; 32:180-9.
49. Luoto K R, Kumareswaran R, Bristow R G. Tumor hypoxia as a driving force in genetic instability. Genome Integr. 2013; 4(1):5.
50. Maybury B D. Mitochondrial DNA damage is uncommon in cancer but can promote aggressive behaviour. Anticancer Res. 2013; 33(9):3543-52.
51. Kim J S, Qian T, Lemasters J J. Mitochondrial permeability transition in the switch from necrotic to apoptotic cell death in ischemic rat hepatocytes. Gastroenterology. 2003; 124(2):494-503.
52. Kim J S, He L, Lemasters J J. Mitochondrial permeability transition: a common pathway to necrosis and apoptosis. Biochem Biophy Res Commun. 2003; 304(3):463-70.
53. Kim J S, in Y, Lemasters J J. Reactive oxygen species, but not Ca2+ overloading, trigger pH- and mitochondrial permeability transition-dependent death of adult rat myocytes after ischemia-reperfusion. Am J Physiol Heart Circ Physiol. 2006; 290(5):H2024-34.
54. Kim J S, Ohshima S, Pediaditakis P. Lemasters J J. Nitric oxide protects rat hepatocytes against reperfusion injury mediated by the mitochondrial permeability transition. Hepatology. 2004; 39(6):1533-43.
55. Kon K, Kim J S, Jaeschke H, Lemasters J J. Mitochondrial permeability transition in acetaminophen-induced necrosis and apoptosis of cultured mouse hepatocytes. Hepatology. 2004; 40(5):1170-9.
56. Kim J S, He L, Qian T, Lemasters J J. Role of the mitochondrial permeability transition in apoptotic and necrotic death after ischemia/reperfusion injury to hepatocytes. Curr Mol Med. 2003:3(6):527-35.
57. Kim J S, Nitta T, Mohuczy D, O'Malley K A, Moldawer L L, Dunn W A, Jr., Behms K E. Impaired autophagy: A mechanism of mitochondrial dysfunction in anoxic rat hepatocytes. Hepatology. 2008; 47(5):1725-36.
58. Wang J H, Ahn I S, Fischer T D, Byeon I, Dunn W A, Jr., Behms K E, Leeuwenburgh C, Kim J S. Autophagy suppresses age-dependent ischemia and reperfusion injury in livers of mice. Gastroenterology 2011; 141(6):2188-99 e6.
59. Kim J S, Wang J H, Biel T G, Kim D S, Flores-Toro J A, Vijayvargiya R, Zendejas I, Behrns K E. Carbamazepine suppresses calpain-mediated autophagy impairment after ischemia/reperfusion in mouse livers. Toxicol Appl Pharmacol. 2013; 273(3):600-10.
60. Biel T G, Lee S, Flores-Toro J A, Dean J W, Go K L, Lee M H, Law B K, Law M E, Dunn W A, Jr., Zendejas I, Behms K E, Kim J S. Sirtuin I suppresses mitochondrial dysfunction of ischemic mouse livers in a mitofusin 2-dependent manner. Cell Death Differ. 2015).
61. DePristo M A, Banks E, Poplin R, Garimella K V, Maguire J R, Hartl C, Philippakis A A, del Angel G, Rivas M A, Hanna M, McKenna A, Fennell T J, Kemytsky A M, Sivachenko A Y, Cibulskis K, Gabriel S B, Altshuler D, Daly M J. A framework for variation discovery and genotyping using next-generation DNA sequencing data. Nat Genet. 2011; 43(5):491-8.
62. Kilgore J A, Hoose S A, Gustafson T L, Porter W, Kladde M P. Single-molecule and population probing of chromatin structure using DNA methyltransferases. Methods. 2007; 41(3):320-32.
63. Kelly T K, Liu Y, Lay F D, Liang G, Berman B P, Jones P A. Genome-wide mapping of nucleosome positioning and DNA methylation within individual DNA molecules. Genome Res. 2012; 22(12):2497-506.
64. Pondugula S. Kladde M P. Single-molecule analysis of chromatin: changing the view of genomes one molecule at a time. J Cell Biochem. 2008; 105(2):330-7.
65. Xu M, Kladde M P, Van Etten J L, Simpson R T. Cloning, characterization and expression of the gene coding for a cytosine-5-DNA methyltransferase recognizing GpC. Nucl Acid Res. 1998; 26(17):3961-6.
66. Darst R P, Haecker I, Pardo C E, Renne R, Kladde M P. Epigenetic diversity of Kaposi's sarcoma-associated herpesvirus. Nucl Acid Res. 2013; 41(5):2993-3009.
67. Blaber E, Marcal H, Burns B P. Bioastronautics: the influence of microgravity on astronaut health. Astrobiology. 2010:10(5):463-73.
68. Li K, Huang T, Zheng J, Wu K, Li D. Effect of marine-derived n-3 polyunsaturated fatty acids on C-reactive protein, interleukin 6 and tumor necrosis factor alpha: a meta-analysis. PLoS One. 2014; 9(2):e88103.
69. National Council on Radiation Protection and Measurements. Radiation Protection Guidance for Activities in Low-Earth Orbit. 2000. Bethesda, Md.: NCRP Report No. 132.
70. International Commission on Radiation Protection. Relative Biological Effectiveness, Radiation Weighting and Quality Factor. Ann ICRP. 2003; 33(4).
71. Adewoye A B, Lindsay S J, Dubrova Y E, Hurles M E. The genome-wide effects of ionizing radiation on mutation induction in the mammalian germline. Nat Commun. 2015; 6:6684.
72. Mouse Tumor Biology Database. Tumor Frequency Grid (Inbred Strain Family×Organ). 2015. Available from: http://tumor.informatics.jax.org/mtbwi/dynamicGrid.do?organKey=|60|&currentOrganKey=|60|&strainFamilyKey=&currentStrainFamilyKey=&expandColapse=false&organGrpChk=18 &organGrpChk=15&organGrpChk=193&organGrpChk=199&organGrpChk=363&organGrpChk=17&organGrpChk=26&organGrpChk=245&organGrpChk=14&organGrpChk=9&organGrpChk=159&organGrpChk=31&organGrpChk=56&organGrpChk=28&organGrpChk=251&organGrpChk=58&organGrpChk=29&organGrpChk=60&organGrpChk=117&organGrpChk=66&organGrpChk=92&organGrpChk=119&organGrpChk=5&organGrpChk=348&organGrpChk=45&organGrpChk=130&organGrpChk=3&organGrpChk=25&organGrpChk=342 &organGrpChk=129&organGrpChk=32&organGrpChk=157&organGrpChk=6&organGrpChk=194&organGrpChk=140&organGrpChk=11&organGrpChk=39&organGrpChk=112&organGrpChk=40&organGrpChk=120&organGrpChk=380&organGrpChk=254&organGrpChk=118&organGrpChk=4&organGrpChk=71&organGrpChk=116&organGrpChk=12&organGrpChk=95&organGrpChk=2&organGrpChk=153&organGrpChk=61&organGrpChk=530&organGrpChk=132&organGrpChk=242&organGrpChk=108&organGrpChk=269&organGrpChk=36&organGrpChk=156&organGrpChk=177&organGrpChk=94&strainFamilyChk=2&strainFamilyChk=70&strainFamilyChk=9&strainFamilyChk=12&strainFamilyChk=4&strainFamilyChk=32&strainFamilyChk=23&strainFamilyChk=43&strainFamilyChk=44&strainFamilyChk=58&strainFamilyChk=112&strainFamilyChk=86&strainFamilyChk=84&strainFamilyChk=87&strainFamilyChk=31&strainFamilyChk=30&strainFamilyChk=29&strainFamilyChk=26&strainFamilyChk=27&strainFamilyChk=28&strainFamilyChk=73&strainFamilyChk=126&strainFamilyChk=116&strainFamilyChk=54&strainFamilyChk=82&strainFamilyChk=83& strainFamilyChk=100&strainFamilyChk=101 &strainFamilyChk=20&strainFamilyChk=3 6&strainFamilyChk=94&strainFamilyChk=65&strainFamilyChk=145&showAllStrains=showAllStrains.
73. Link A, Lenz M, Legner D, Bohm M, Nickenig G. Telmisartan inhibits beta2-integrin MAC-1 expression in human T-lymphocytes. J Hypertens. 2006; 24(9):1891-8.
74. Moertl D, Hammer A, Steiner S Hutuleac R, Vonbank K, Berger R. Dose-dependent effects of omega-3-polyunsaturated fatty acids on systolic left ventricular function, endothelial function, and markers of inflammation in chronic heart failure of nonischemic origin: a double-blind, placebo-controlled, 3-arm study. Am Heart J. 2011; 161(5):915 e1-9.
75. Nodari S, Triggiani M, Campia U, Manerba A, Milesi G, Cesana B M, Gheorghiade M, Dei Cas L. Effects of n-3 polyunsaturated fatty acids on left ventricular function and functional capacity in patients with dilated cardiomyopathy. J Am Coll Cardiol. 2011; 57(7):870-9.
76. Trevelyan J, Brull D J, Needham E W, Montgomery H E, Morris A, Mattu R K. Effect of enalapril and losartan on cytokines in patients with stable angina pectoris awaiting coronary artery bypass grafting and their interaction with polymorphisms in the interleukin-6 gene. Am J Cardiol. 2004; 94(5):564-9.
77. Pavlatou M G, Mastorakos G, Margeli A, Kouskouni E, Tentolouris N, Katsilambros N, Chrousos G P, Papassotiriou I. Angiotensin blockade in diabetic patients decreases insulin resistance-associated low-grade inflammation. Eur J Clin Invest. 2011; 41(6):652-8.
78. Medhora M, Gao F, Wu Q, Molthen R C, Jacobs E R, Moulder J E, Fish B L. Model development and use of ACE inhibitors for preclinical mitigation of radiation-induced injury to multiple organs. Radiat Res. 2014; 182(5):545-55.
79. Moulder J E, Cohen E P, Fish B L. Mitigation of experimental radiation nephropathy by renin-equivalent doses of angiotensin converting enzyme inhibitors. Int J Radiat Biol. 2014; 90(9):762-8.
80. Moulder J E. 2013 Dade W. Moeller lecture: medical countermeasures against radiological terrorism. Health Phys. 2014; 107(2):164-71.
81. Hesson L B, Pafil V, Sloane M A, Nunez A C, Liu J, Pimanda J E, Ward R L. Reassembly of nucleosomes at the MLH1 promoter initiates resilencing following decitabine exposure. PLoS Genet. 2013; 9(7):e1003636.
82. Marcus C S. Time to Reject the Linear-No Threshold Hypothesis and Accept Thresholds and Hormesis: A Petition to the U.S. Nuclear Regulatory Commission. Clin Nucl Med. 2015; 40(7):617-9.
83. Lehrer S. Rosenzweig K E. Lung cancer hormesis in high impact states where nuclear testing occurred. Clin Lung Cancer. 2015; 16(2):152-5.
84. Morgan W F, Bair W J. Issues in low dose radiation biology: the controversy continues. A perspective. Radiat Res. 2013; 179(5):501-10.
85. Varley K E, Mitra R D. Bisulfite Patch PCR enables multiplexed sequencing of promoter methylation across cancer samples. Genome Res. 2010; 20(9):1279-87.
86. Weidner C I, Lin Q, Koch C M, Eisele L, Beier F, Ziegler P, Bauerschlag D O, Jockel K H, Erbel R, Muhleisen T W, Zenke M, Brummendorf T H, Wagner W. Aging of blood can be tracked by DNA methylation changes at just three CpG sites. Genome Biol. 2014; 15(2):R24.
87. Naugler W E, Karin M. The wolf in sheep's clothing: the role of interleukin-6 in immunity, inflammation and cancer. Trends Mol Med. 2008; 14(3):109-19.
88. Barcellos-Hoff M H. How do tissues respond to damage at the cellular level? The role of cytokines in irradiated tissues. Radiat Res. 1998; 150(5 Suppl):S109-20.
89. Manning C M, Johnston C J, Reed C K, Lawrence B P, Williams J P, Finkelstein J N. Lung irradiation increases mortality after influenza A virus challenge occurring late after exposure. Int J Radiat Oncol Biol Phys. 2013; 86(1):128-35.
90. Johnston C J, Hernady E, Reed C, Thurston S W, Finkelstein J N, Williams J P. Early alterations in cytokine expression in adult compared to developing lung in mice after radiation exposure. Radiat Res. 2010; 173(4):522-35.
91. Gridley D S, Dutta-Roy R, Andres M L, Nelson G A, Pecaut M J. Acute effects of iron-particle radiation on immunity. Part II: Leukocyte activation, cytokines and adhesion. Radiat Res. 2006; 165(1):78-87.
92. Pecaut M J, Dutta-Roy R, Smith A L, Jones T A, Nelson G A, Gridley D S. Acute effects of iron-particle radiation on immunity. Part I: Population distributions. Radiat Res. 2006; 165(1):68-77.
93. Dong C, He M, Tu W, Konishi T, Liu W, Xie Y, Dang B, Li W, Uchihori Y, Hei T K, Shao C. The differential role of human macrophage in triggering secondary bystander effects after either gamma-ray or carbon beam irradiation. Cancer Lett. 2015; 363(1):92-100.
94. Chatterjee A, Borak T H. Physical and biological studies with protons and HZE particles in a NASA supported research center in radiation health. Phys Med. 2001; 17 Suppl 1:59-66.
95. Chen Y, Williams J, Ding I, Hernady E, Liu W, Smudzin T, Finkelstein J N, Rubin P, Okunieff P. Radiation pneumonitis and early circulatory cytokine markers. Semin Radiat Oncol. 2002; 12(1 Suppl 1):26-33.
96. Evans E S, Kocak Z, Zhou S M, Kahn D A, Huang H, Hollis D R, Light K L, Anscher M S, Marks L B. Does transforming growth factor-beta1 predict for radiation-induced pneumonitis in patients treated for lung cancer? Cytokine. 2006; 35(3-4):186-92.
97. Zhang H, Maguire D J, Zhang M, Zhang L, Okunieff P. Elevated mitochondrial DNA copy number and POL-gamma expression but decreased expression of TFAM in murine intestine following therapeutic dose irradiation. Adv Exp Med Biol. 2011; 701:201-6.
98. Zhang H, Maguire D, Swarts S, Sun W, Yang S, Wang W, Liu C, Zhang M, Zhang D, Zhang L, Zhang K, Keng P, Okunieff P. Replication of murine mitochondrial DNA following irradiation. Adv Exp Med Biol. 2009; 645:43-8.
99. Kim J S, Lemasters J J. Opioid receptor-independent protection of ischemic rat hepatocytes by morphine. Biochem Biophys Res Commun. 2006; 351(4):958-64.
100. Trovati S, Ballarini F, Battistoni G, Cerutti F, Fasso A, Ferrari A, Gadioli E, Garzelli M V, Mairani A, Ottolenghi A, Paretzke H G, Parini V, Pelliccioni M, Pinsky L, Sala P R, Scannicchio D, Zankl M. Human exposure to space radiation: role of primary and secondary particles. Radiat Prot Dosim. 2006; 122(1-4):362-6.
101. Badhwar G D, O'Neill P M. Response of silicon-based linear energy transfer spectrometers: implication for radiation risk assessment in space flights. Nucl Instrum Methods Phys Res A. 2001; 466(3):464-74.
102. Bahadori A A, Sato T, Slaba T C, Shavers M R, Semones E J, Van Baalen M, Bolch W E. A comparative study of space radiation organ doses and associated cancer risks using PHITS and HZETRN. Phys Med Biol. 2013; 58(20):7183-207.
103. Armstrong T W, Colbom B L. Predictions of secondary neutrons and their importance to radiation effects inside the international space station. Radiat Meas. 2001; 33:229-34.
104. Koshiishi H, Matsumoto H, Chishiki A, Goka T, Omodaka T. Evaluation of the neutron environment inside the International Space Station based on the bonner ball neutron detector experiment. Radiat Meas. 2007:42:1510-20.
105. Mitchell J B, Bedford J S, Bailey S M. Dose-rate effects in mammalian cells in culture III. Comparison of cell killing and cell proliferation during continuous irradiation for six different cell lines. Radiat Res. 1979; 79(3):537-51.
106. Yi P N, Stanley W S, Lee W. Relationship between mitotic delay and the minimum dose rate of X irradiation required to stop cell proliferation. Radiat Res. 1993; 133(2):163-9.
107. Williams J P, Brown S L, Georges G E, Hauer-Jensen M, Hill R P, Huser A K, Kirsch D G, Macvittie T J, Mason K A, Medhora M M, Moulder J E, Okunieff P Otterson M F, Robbins M E, Smathers J B, McBride W H. Animal models for medical countermeasures to radiation exposure. Radiat Res. 2010; 173(4):557-78.
108. Williams J P, Jackson I L, Shah J R, Czarniecki C W, Maidment B W, DiCarlo A L. Animal models and medical countermeasures development for radiation-induced lung damage: report from an NIAID Workshop. Radiation research. 2012; 177(5):e0025-39.
109. Doll R. The Age Distribution of Cancer: Implications for Models of Carcinogenesis. J R Stat Soc Series A. 1971; 134(2):133-66.
110. Frank S A. Dynamics of Cancer. Princeton, N.J.: Princeton University Press; 2007.
111. Cucinotta F A. Biophysics of Nasa Radiation Quality Factors. Radiat Prot Dosim. 2015).
112. Cucinotta F A. A new approach to reduce uncertainties in space radiation cancer risk predictions. PLoS One. 2015; 10(3):e0120717.
113. Sridharan D M, Asaithamby A, Bailey S M, Costes S V, Doetsch P W, Dynan W S, Kronenberg A, Rithidech K N, Saha J, Snijders A M, Werner E, Wiese C, Cucinotta F A, Pluth J M. Understanding cancer development processes after HZE-particle exposure: roles of ROS, DNA damage repair and inflammation. Radiat Res. 2015; 183(1):1-26.
114. Vassetzky et al. (2012), SINEBase: a database and tool for SINE analysis, Nucl. Acid Res., 1-7.
115. Baker (2012), Digital PCR hits its stride, Nature Methods, 9:541-544.
116. Hundson et al. (2011), High-Throughput Droplet Digital PCR System for Absolute Quantitation of DNA Copy Number, Anal. Chem., 83:8604-8610.

1. An assay to determine the number of accumulated mutations m a target sequence within a target short interspersed element (SINE), target long interspersed element (LINE) and/or the genome of a subject, the assay comprising the steps of:
a) obtaining a genomic DNA sample from the subject and fragmenting the genomic DNA sample, or obtaining a fragmented genomic DNA sample from the subject,
b) mixing a predetermined number of fragments of the genomic DNA that arise from a predetermined number of genomes with a reagent mixture to produce a reaction mixture, wherein the reagent mixture comprises:
i) a pair of polymerase chain reaction primers that amplify a target amplicon comprising the target sequence within the target SINE, target LINE and/or target sequence within the genome,
ii) a target sequence clamp which binds only to the wild-type target sequence within the target SINE, target LINE and/or target sequence within the genome, wherein the target sequence clamp prevents the PCR amplification of only those target amplicons that have the target wild-type sequence within the target SINE, target LINE and/or target sequence within the genome and permits the PCR amplification of only those target amplicons that have the target mutated sequence within the target SINE, target LINE and/or target sequence within the genome, and
iii) a DNA polymerase enzyme and the reactants for a digital PCR (dPCR),
c) subjecting the reaction mixture to the dPCR,
d) identifying the number of fragments of the genomic DNA comprising the target amplicon having the target mutated sequence within the SINE, target LINE and/or target sequence within the genome based on the number of positive PCR amplifications in the dPCR, and
e) calculating the number of accumulated mutations per genome m the target sequence within the target SINE, target LINE and/or target sequence within the genome based on the number of fragments of the genomic DNA that arise from one genome and the number of fragments of the genomic DNA per genome that comprise the target amplicons having the target mutated sequence within the target SINE, target LINE and/or target sequence within the genome wherein the presence of the target mutated sequence within the target SINE, target LINE and/or target sequence within the genome is indicated by PCR amplification of the target amplicon in the dPCR.

2. The assay of claim 1, wherein the target SINE, target LINE and/or target sequence within the genome is about 50-500, about 100-400, about 100-250, about 200-300, about 250-350 or about 300 bp.

3. The assay of claim 1, wherein the target SINE, target LINE and/or target sequence within the genome covers about 4%-15% of the genome of the subject.

4. The assay of claim 1, wherein each genomic DNA fragment from at least about 90% of the genomic DNA fragments is about 800-1500 bp.

5. The assay of claim 1, wherein the reactants for a digital PCR comprise deoxyribonucleotides (dNTPs), a metal ion selected from Mg2+ or Mn2+ and a buffer.

6. The assay of claim 1, wherein the target sequence clamp is about 20 bp.

7. The assay of claim 1, wherein the melting temperature of the target sequence clamp with the target sequence is higher than the temperatures used in the PCR cycle.

8. The assay of claim 1, wherein the target sequence clamp comprises xenonucleotide (XNA).

9. The assay of claim 1 wherein the dPCR is droplet dPCR (ddPCR).

10. The assay of claim 1, wherein the subject is a mammal.

11. The assay of claim 10, wherein the mammal is a human, non-human primate, rat, mouse, pig, dog or cat.

12. The assay of claim 11, wherein the mammal is a mouse and the target SINE is B1 which has the sequence of SEQ ID NO: 6.

13. The assay of claim 12, wherein the primer pair comprises the sequences of SEQ ID NOs: 1 and 2 and the target sequence clamp has the sequence of SEQ ID NOs: 3, 4 or 5.

14. The assay of claim 10, wherein the mammal is a human and the target SINE is Alu which as the sequence selected from of SEQ ID NOs: 11-98.

15. The assay of claim 14, wherein the primer pair comprises the appropriate sequences selected from SEQ ID NOs: 99-274 and the target sequence clamp has the appropriate sequence selected from SEQ ID NOs: 7-10.

16-29. (canceled)

30. A kit comprising:
a) a pair of PCR primers that amplify a target amplicon comprising a target sequence within a target SINE, target LINE and/or target sequence within the genome,
b) a target sequence clamp which binds only to the wild-type target sequence within the SINE, target LINE and/or target sequence within the genome, wherein the target sequence clamp prevents the PCR amplification of only those target amplicons that have the target wild-type sequence within the SINE, target LINE and/or target sequence within the genome and permits the PCR amplification of only those target amplicons that have the target mutated sequence within the SINE, target LINE and/or target sequence within the genome.

31. The kit of claim 30, the kit further comprising a DNA polymerase enzyme and reactants for conducting a dPCR.

32. The method or kit according to any preceding claim wherein the target sequence within the genome is a highly repeated genomic sequence or mitochondrial genomic DNA.


SEQUENCE LISTING (501 sequences)

SEQ ID NO: 1 - DNA, 14 bp, Mus musculus
cctgtaatcccagc

SEQ ID NO: 2 - DNA, 20 bp, Mus musculus
ctttaatcccagcactcggg

SEQ ID NO: 3 - DNA, 20 bp, Mus musculus
ctcagaaatccgcctgcctc

SEQ ID NO: 4 - DNA, 16 bp, Mus musculus
ggctggcctcgaactc

SEQ ID NO: 5 - DNA, 20 bp, Mus musculus
gtgtcctggaactcactctg

SEQ ID NO: 6 - DNA, 144 bp, Mus musculus
gccgggcatggtggcgcacgcctttaatcccagcactcgggaggcagaggcaggcggatttctgagttcgaggccagcctggtctacagagtgagttccaggacaccagggctacagagagaaaccctgtctcgaaaaaaaaaa

SEQ ID NO: 7 - DNA, 14 bp, Homo sapiens
cctgtaatcccagc

SEQ ID NO: 8 - DNA, 14 bp, Homo sapiens
ctaaaaatacaaaa

SEQ ID NO: 9 - DNA, 14 bp, Homo sapiens
tgcactccagcctg

SEQ ID NO: 10 - DNA, 14 bp, Homo sapiens
tctcaaaaaaaaaa

SEQ ID NO: 11 - DNA, 136 bp, Homo sapiens
ggccagccacagtgctaatgcctgtaatcccagccctttgggatcctagcacttagagcccagagttccaaccagaccagggtttcaagaaagtgaaactgtctctacccaaaataccaaaattggccagtcttag

SEQ ID NO: 12 - DNA, 172 bp, Homo sapiens
actaaaatcaaattagcagcacagtgcacatgcctgtaatcccagctacttgggaggctgaggcagtagaatcacttgacctggaggtggaggttgcgtgagctgaggtcgcaccattgcactccagcctgggcaacaagggcaaaacccgtctaaaaaaaaaaaaaaaaaa

SEQ ID NO: 13 - DNA, 190 bp, Homo sapiens
aaaaattagccagccgtggtggcccgtgcctgtaatcccagctactcagaggctgaggcaggagaatcgcttaaacccggaaggcggaggttgcagtggccgagattgccgccgttgcactctagcctgggcgacagagcgagactgctcaaaaaaaactaaacaaaacaaaaaaagttattagattaaa

SEQ ID NO: 14 - DNA, 168 bp, Homo sapiens
aaaagtaaaaagaggggccaggcacagtggctcagcctgtaatcccagcactttgggaggctgaggtgggcaggatcacctgagctcgggaagttgaggctaatagtgggctgagattgtgccactgcactccagcctgggtgacagggaaggagaccctgtctcaaa

SEQ ID NO: 15 - DNA, 199 bp, Homo sapiens
cccggggatcgcttgattccaggatgataccagcctggccaacgtggtgaaaccacatctctactaaaaatacaaaaactagccaggcattctatcgcacgcctgtaatcccagctacttgggagggctgaggaacaagaatagcttgaacccaggcggtggaagtttgcagtgagccgagatctttgaggacaccgca

SEQ ID NO: 16 - DNA, 207 bp, Homo sapiens
gagtgactcacttgaggtaacagcctacatacatgaccataaatgtggccaaacatcccactcagaaatattcatcataagccaggcgtggtggcatacacctgtaatcccagctactcaggaggctgaggtaggagaatcccatgagcccatgaccgcacccctgcacaccagcctgggtgacagatggagatcctgtctcaaaaa

SEQ ID NO: 17 - DNA, 166 bp, Homo sapiens
ggccaggcatggtgctcatcgcctgtaatcccagcactttgggaggccgagaaagatggatgaagtcaggagttcaagaccagcctgggcaacatggcagaaccccgtctctactaaaaatacaaaaaattagccgggcgtggtggtgggcgcctgtaatcccagc

SEQ ID NO: 18 - DNA, 167 bp, Homo sapiens
ccctttataacatttaaattaggttgggcacagtggctcacacctgtaatcccagcattttgggagcccaagataggaggatagattgagcccaggaggtcaaggctgcagtgagccaagatcatgttgctgcattccagcctaaaagagcaagaccccatctgaaa

SEQ ID NO: 19 - DNA, 199 bp, Homo sapiens
atttaagagttgagtagggctgtgtgtggcgttctcgcctgtaatcccagcactttgggaggttgaggcaagaggattgtggcaggaggattgcttgagccccagaggtcagggctatagtgagctatcatcgtaccactgcactccagcctggatgatagagtgagacccttctctattaaaaaaaaaaaaaaaaaaa

SEQ ID NO: 20 - DNA, 233 bp, Homo sapiens
gatcacgtgaggtcaggagttcgtgagtagcctggccagcatggtgaaaccccatctctactaaaaatacacacacacacacacacacacacacacacacacacacacacacacaattagccaggcatggtggcttacgcctgtaatcccagctactagggaggctgagacaggagaattgctggaaccaggaggcagaggctgcagtgagctgagatgcgcacagcatccag

SEQ ID NO: 21 - DNA, 232 bp, Homo sapiens
cagtacttttgggaggccaggtcggtgaccgtgggatcactgaggttaggggttcaagaccagcctggccaacatggcgaaactcgcctctactaaaaatacaaaacttagccgggcgtgatggtgggcacctgtaatcccagctattcgggaggcagaggttgcaagtcatgccactgcaccccagcctaggcgacagagcaagactccattaaaaaaaaaaaaaaaagaa

SEQ ID NO: 22 - DNA, 269 bp, Homo sapiens
cccaggcactttgggagggctgaggcgggcagaccacctgaggtcaggagtttgagaccagcctgaccaatgtgatgaaaccctgtctctacttcaaacacacacacacacacacacacacacacacacacacacacacacacacagtcagctgggcgtggtggcatgtgcctgtaatcccagctactcgggaggctgagacaggagaatcgcttgaattcggtgttgtggggagagggggttggggaaggttgcagtgagccgagatc

SEQ ID NO: 23 - DNA, 255 bp, Homo sapiens
ggtggctcacgcctgtaatcccagcactttgttaggccgaggcgggtggatcacttgaggtcagtgactagcctgacaaacatggtaaaacttcgtctctgccaaaataacacaaacattagccgggtgttgtggcgggctcctgtaatcccagctactcgagaggctgaggcaggagaatcgcttgaacccgggagggtagtttgcagtgagccgagatagggccactgcactccagtctggaaaaaaacaaaa

SEQ ID NO: 24 - DNA, 180 bp, Homo sapiens
aaggttagtgattcttggccgggtgtgatggctcacacctgtaatcccagcattttggaaggctgagggaggctgatcctttgaggtctagctgcgcatgatggtgggtgcctgtaatcccagctactcaggctgaggcaggagaatcgcttgagcccgggaggtggaggttgcagtgag

SEQ ID NO: 25 - DNA, 280 bp, Homo sapiens
aaggttagtgattcttggccgggtgtgatggctcacacctgtaatcccagcattttggaaggctgagggaggctgatcctttgaggtctagctgcgcatgatggtgggtgcctgtaatcccagctactcaggctgaggcaggagaatcgcttgagcccgggaggtggaggttgcagtgagcagagattgcaccactacattccagccagcctgggtgacagagcaaggctccatctcaaaaaaataaaaagaaaaagaaaccacaaagggttttaaagct

SEQ ID NO: 26 - DNA, 180 bp, Homo sapiens
tggccaacatggcaaaaccccatctctactgaaaacaaaactagaagagcgtggtggtgtgcacctgtaatcccagctacttgggaggctgaggagaattgcttaagcctgggaggtggaggttcgagtgagccgagatctcgaccactgcactccagcctagcgacagagcaagactct

SEQ ID NO: 27 - DNA, 180 bp, Homo sapiens
tggccgacacggtgaaacccaccagtctctactaaaaatacaaaaattagcctgtaatcccagctactcaggagactgaggcaggagagtcgcttgaacccaggaggtggggttgcagtgagccaagatcgcacaactacactccagcctgggagacaagagtggaactccgtcagtaaa

SEQ ID NO: 28 - DNA, 180 bp, Homo sapiens
ccaacatggtgaaaccctgtgtctactaaaaaatgcaaaaaaaaaaattagccaggtgtggtggtgcctgcctgtaatcccagctaatcaggaggctgagacatggaatcgcttgagcacaggaggcaagtttgacgtgagctgagatcacgtcattgcacgccagcctgggcgacagag

SEQ ID NO: 29 - DNA, 180 bp, Homo sapiens
ccccgtctctactaaaaatacaaaaaaaaaaaaaaaaaaaaaaaattagccgggcatggtggcacatgcctgtaatcccagctactaaggaggcaggagaatcgcttgaacccgggagggggaggtgcagtgaactgagatcgcgccattgcactccagcctgggtaataagagcgaaac

SEQ ID NO: 30 - DNA, 180 bp, Homo sapiens
ctagccaacatggtgtaaacactgtctctactaaaaatacaattagccaggtgtggcggcgtaatcccatgctaacctgtaatcccagctacttgggaggctgaggcagagctgcttgaaccctggaagtggaggttgcagtgagcctgtcatcgctccactgagccaagatcgctccca

SEQ ID NO: 31 - DNA, 180 bp, Homo sapiens
tcaacatgatgaaaccccacctctactgaaaatacaaaaattagcccggtgcagtggcgggcacctgtaatcccagctacttcggaggctgaggtgggagaattgcttgaacccgggagcagaggttgcagtgagctgagatggtgcccctgccattcagcctgggtgacagagcaagac

SEQ ID NO: 32 - DNA, 180 bp, Homo sapiens
tgaccaacacggagaaaccccttctctactaaaaacacaaaattagctgggcgtggtggcgcatgcctgtaatcccagctactcgggaggctgcggcaggagaatcgcttgaatccaggaggcggaggttgtggtgagctgagatcacgtcattgcactccagcctgggacaacaagagt

SEQ ID NO: 33 - DNA, 161 bp, Homo sapiens
gcggtgactcacgcctgtaatcccagcacttcgggaggccaaggcgggcggttcacctgaggtcaggagttcgaaaccagtctggcaaacatggtgaaaaccccgtctctactaaaaatacaaaagtagcggggtgtcgtggcgtatgctggtaatcctag

SEQ ID NO: 34 - DNA, 180 bp, Homo sapiens
cccggggatcgcttgattccaggatgataccagcctggccaacgtggtgaaaccacatctctactaaaaatacaaaaactagccaggcattctatcgcacgcctgtaatcccagctacttgggagggctgaggaacaagaatagcttgaacccaggcggtggaagtttgcagtgagccga

SEQ ID NO: 35 - DNA, 180 bp, Homo sapiens
gccgggcaccggttcacgcctgtaattccaggcactctgggagcgcccaggtggccggcacacaaggatcaggagattgagaccatcctggctaacacggtgaaaccctgtctctactaaaaatacaaaaaattagccggggcgcggtggtgggcgcctgtagtcacagctacttgggag

SEQ ID NO: 36 - DNA, 166 bp, Homo sapiens; identical to SEQ ID NO: 17

SEQ ID NO: 37 - DNA, 180 bp, Homo sapiens
gccgggcacgtggctcacgcctgtaatctcagcactttgggaagccaaggtgggtggatcactgaggttgggcgttcgtgactagcctgatcaacatggagaaacctggtctctactaaaaatacaaaattagccgggcatgattgtgcatgctgtaatcccagctactcgggaggctga

SEQ ID NO: 38 - DNA, 180 bp, Homo sapiens
ggccgggaacgtggctcacacctgtaatcccagcactttcagtggcggagcacctgtggccaagagtttgagaccagcctggccaacatggtgaaaccccatctctactaaaaatacaaaaattgaggtgccgctgttgctgctcgtgttaaatctagaaccgtagccagacatgggact

SEQ ID NO: 39 - DNA, 180 bp, Homo sapiens
agctgggcacgtggctcacccctgtgatcccagcactttgggaggctagaggtgggtggatcgcctcaactcctgacagagtttaagaccagcctggccaacatagtcaaagcccgtctctactaaaaatacaaaatattagctaatcgtggtggtgggcgcctgtaatcccgctacccg

SEQ ID NO: 40 - DNA, 180 bp, Homo sapiens
cagtacttttgggaggccaggtcggtgaccgtgggatcactgaggttaggggttcaagaccagcctggccaacatggcgaaactcgcctctactaaaaatacaaaacttagccgggcgtgatggtgggcacctgtaatcccagctattcgggaggcagaggttgcaagtcatgccactgc

SEQ ID NO: 41 - DNA, 180 bp, Homo sapiens
ggcgggcgggatcacgaggtcaggacatcagatccatcctgactaactcggtgaaacccgtctctactaaaaatacaaaaaattaaccaggcgtagtggcgggcctgtagtcccagctactcgggaggctgaggcaggagaatggcgtgaacccgggagcggacttgccgtgagccagat

SEQ ID NO: 42 - DNA, 180 bp, Homo sapiens
agccaggcttggtggctcacacccgtaatcccagcacttaggctaaggcaggcggatcacaacatctagagatcctggccaacgcggtgaaaccctgtctgtactaaaaatacaaaataactgggcatggtggtgtgcacctgtgggaggctgaggcagaattgctggaacccgggagat

SEQ ID NO: 43 - DNA, 180 bp, Homo sapiens
ggccaagcgctggtggctcacacctgtaatctcagcactctgggaggctgaggcgggcagatcacaaggtctctactaaaaatacaaaaaaaaaaaaaaatggcccagcttggtggcgtgtgcctgtaatcccaactactcgggaggctgaggcaggagggtgcagtgagctgagatcgt

SEQ ID NO: 44 - DNA, 180 bp, Homo sapiens
ggccaggtgcagtgctcacgcctcacgcccgcactctgggaatccgaggtgggcggatcacttgtggccatgagtttgaaccagcctggctaacatggcgaaaccccgtctctactaaaaatacaaaatcagccaggcatgtcagtggacgcctgtaatcaatcccagctacttgggagc

SEQ ID NO: 45 - DNA, 180 bp, Homo sapiens
gtaatcccagcactaggcgggtgcatcacctgacatcaggagttgaataccagcctggccaacatggcaaaaccctgtctctactaaaaatacaaaaaatacaaaagaaaattagctgggcgtgttggcatgggcctgtaacccaactacacggaagctgaggcaggagaatcgcttcaa

SEQ ID NO: 46 - DNA, 180 bp, Homo sapiens
ggccaggcacagtggctcatgcctgaaatcccaacactgggacgccagggtgggcagatcacttgaggccaacttgagtttgagactagcctggccaacatagtgaaaccccgtctctgctaaaaatacaaaaattcgcgcgcctgtggtcccagctctggggctgaggaatgggaactc

SEQ ID NO: 47 - DNA, 180 bp, Homo sapiens
gtcgtgtgtggtggctcatacctataatccagcaccttgggaggccgaggcaggcagatcacttgaggccaggagtttgagaccagcctggccaacatcatgaaacccccgtgtctactaaaaatacaaaaaaattagctggggatggtggcgggcgcctgtaatctcagctacttggga

SEQ ID NO: 48 - DNA, 180 bp, Homo sapiens
ggctgggcacagacgttcacgcctgtggtctcggcactttgggaggccgaggcgggtgaatcacttgaggtcaggggttcgagaccaacctggccgacacggtgaaacccaccagtctctactaaaaatacaaaaattagcctgtaatcccagctactcaggagactgaggcaggagagt

SEQ ID NO: 49 - DNA, 180 bp, Homo sapiens
ggccgggtgtggtggctcaagcctgtaatcctagcactttgggagctgaggtgggcgcccaggccaggggtttgagaccatcttggccaacatagtgaaacccccaacctctactaaaaatacaaaaattagctggggtgtggtggcacgcgcctgtaatcctagctactagggaggctg

SEQ ID NO: 50 - DNA, 180 bp, Homo sapiens
ctgtaatcccagcactttgggaggtcacaaagtgacctcaggatcagctgaggtcaggagtttgagaccagcctggccaacatggtgaaaccccgtctctactaaaaatacaaaaaaaaaaaaaaaaaaaaaaaattagccgggcatggtggcacatgcctgtaatcccagctactaagg

SEQ ID NO: 51 - DNA, 180 bp, Homo sapiens
ggccgggtgcagtggcttatgcctgtaatcccagcactttgagaggtcgaggcaggcagctctctcaagttcaggagttcaagaccagcctggccaacatggtgaagccccgtctctactaaaaatacaaaaattagctgggtgtggtggcatgcacctgtaatcccagctacttgggag

SEQ ID NO: 52 - DNA, 180 bp, Homo sapiens
ggccaggcacagtggctcaggcctgtaatctcagccctttgggaggccggtgggtgggtcacttggtcaggagtttgagaccagcctggccaacatggtgaaagcttcgtctctactaaaaatacaaaaattagctggatgtggtggcatgagcctgtaatcctagctattgaggcagga

SEQ ID NO: 53 - DNA, 180 bp, Homo sapiens
agccaggtgcagtctcacgcctgtaatcctacactttgagagccaaggcaggtgggtcacctgaggtcaagagtttgagaccagcctggccaacacggtgaaaccctgtctctactaaaaatacaaaaataataataataataataataataataattagccaagtgtggtggcaggcac

SEQ ID NO: 54 - DNA, 166 bp, Homo sapiens
ctgggattgcgggcatggtggcgtgagcctgtagtcctagctactcgggaggctgaggcaggagaatagcctgaacctgggaatcggaggttgcagggccaagatcgccccactgcactccagcctggcaatagaccgagctccgtctccaaaaaaaaaaatacaa

SEQ ID NO: 55 - DNA, 164 bp, Homo sapiens
ggtcgctcatgcctataatcccagcactttgggaggcctaggcaggaggaccattgagcccaggaggtccagactgcagtgagccgagattgcgccactgcactccagcctgggcgacagcaagaccttgtctcaaaacaaaagcaaaaacaaaaataaacaaa

SEQ ID NO: 56 - DNA, 180 bp, Homo sapiens
agatgttctcagacaagccaggtgaggtgtctcacgcctgcaatctcagtgctttgggaggccaaagtgggaggatcccttggggccaggagttcgaggatgcagtgagctatgatcgttgccactgcactccagcctgggaaagagagtgagaccctgtctcttaaaaaaaagaaaaaa

SEQ ID NO: 57 - DNA, 172 bp, Homo sapiens; identical to SEQ ID NO: 12

SEQ ID NO: 58 - DNA, 177 bp, Homo sapiens
ccaaaaatgttaaaattagccaggcatagttgtgtgtgcctacagtgctacaggaggctgaggcaagaggattgcttgagttaaggaaggaagtcaaggctgcagtgagctatggtcatgccactgcactccagcctgggcaacagcaagaccctgtgtccaaaaaaaaaaaaggaa

SEQ ID NO: 59 - DNA, 166 bp, Homo sapiens
ctacttgggaggctgaggcaggagaatcacttgaacctgggaggcagagattgcagtgagcctagattgtgccactgcactccagcctgggtgacagagagattctgtcaaaaaaaaaaaaaagcaattccatttataatagcataaaaaaatacttaggaataaa

SEQ ID NO: 60 - DNA, 180 bp, Homo sapiens
gagctcagaccagcctggctaacatggtgaaaccccatctctaccaaaaacacaaatatgggccagccagttgggatgctgaggctccagaatatcctaaacctgggaggcgaaggttgctttgacatcacaccactgcactccagcctgggcgacagagtgaggctgtgtctcaaaaag

SEQ ID NO: 61 - DNA, 186 bp, Homo sapiens
aaaaataataatataagctgggcttgtggctcacacctgtaaccccagagttttgggaggccaaggcttaagcctaggagtttggtgctgcagtgagctatgattgtgccattgcactccagcctgggtgacagagtgagaccctgtttccaaaaaatatataaataaataatacaacaataaaaa

SEQ ID NO: 62 - DNA, 188 bp, Homo sapiens
gcttggtgcgggcgcacctgtaatcccagctactcagggggctgaggcaggagaatcgcttgaagagggcccatggaagttgcagtgagctgagatcactccactgcactccagcctgggtgacagagcgagacgcagtctcaataaaaaaaaaaaaaagggaaagggaaagggaaagggaaagaaca

SEQ ID NO: 63 - DNA, 190 bp, Homo sapiens
aatttataaactggagctgggtgtggtaacaagctcctgtagtcccagctactcaggagactgtggatcgttttgagcccaggagtttgaggctgcagtgagttgtgactgtgccactgcactccagcctgggcaacagagcaagaccctatctctaaagaaataaaaataaatctataatgggaaggag

SEQ ID NO: 64 - DNA, 172 bp, Homo sapiens
tttcagcacttggaaggctgagatggatggacggctggagcccaggaggtcgaggctgcagctgcagtaagctatgactgcgccactgcactccagcctggaagacagagcaagactcagtctcgaaagagagaaaagaaagaaaaagggagagagagagagagaagcaaga

SEQ ID NO: 65 - DNA, 195 bp, Homo sapiens
agatccagccacatgtgaacccgtctgtactaaaatacaaacattagccatgcatgtgcggcacctgagtcccagctactcgagtgagcagagaatcacttgaagccagagcagaggtgtgtgagtgaagtcacagccactgcactccagcctgggtgacagagcgaaactccatttggacaaaaaagaaagaaa

SEQ ID NO: 66 - DNA, 125 bp, Homo sapiens
caatcttctcaattaggacgcccggaagtgggaggatttcttgagcccgggaggttgaggttgcagtgagtggagattgcattgttgcactccagcctgggcaaaagagagagatcctgtctcaa

SEQ ID NO: 67 - DNA, 186 bp, Homo sapiens
aagagggtgaaaccccgtgtctaccaaaaaaaaaaaaaaaaaaaatgagccggccatggtggcgctgagacaggagaatcacttgaacccaggaggcagaggttccagtgagctgagatcacgccactgcactccagcctgggggacagagcaagactccatctcagaaacaaacaaacacacaaa

SEQ ID NO: 68 - DNA, 204 bp, Homo sapiens
ccagcttgaacaacacagcaagaccttgtctctattaaaacttttaaaaaattagccaggtgtggtgcacacgcctttagtcccagctactcgggaggctgaggcaggaggatcatttgagcccaggaggtggaagctgcagtgggctgtgatcatgcccactgcactccagcctgggtgacagagcaagaccttgtgtacaga

SEQ ID NO: 69 - DNA, 194 bp, Homo sapiens
caggtgtggtggcgcatgcctgtaaccccagctacttgggaggctgaggcaggagaatcgaatcgcttgaacctgggaggcagaggctgcagtgagctgagactgcactgcactccagcctgggtgacagagcaagactctgtctcagaaataaataaataaataaataaaataaaattaaaaaaaagaaagag

SEQ ID NO: 70 - DNA, 161 bp, Homo sapiens
aggtgtggtgggacaggcccgtagtccttcctagccacttaggagactaaggcaagaggactgcttgagcccaggaatcccaggttagagagagctatatggcactactgcactccagcctgggcaacagagccaagaccctgtctgtaaaaataaacaaa

SEQ ID NO: 71 - DNA, 167 bp, Homo sapiens
aaaaaaaaaatggcccagcttggtggcgtgtgcctgtaatcccaactactcgggaggctgaggcaggagggtgcagtgagctgagatcgtgccactgcactccagcctgggcagcagagcaagactgtctcgaaaaataaataaataaaataggaagcgacaagaaa

SEQ ID NO: 72 - DNA, 176 bp, Homo sapiens
agtgaaaccccgtctctactaaaaatgtgaaaattagctgggcatggtggaacatgctgtagttccagcttgaacccaggggtggaggttgtagtgagcctagatcacgccactgcactccagcctgagcaaaacagtgagactctgtctaaaaaaaaaaaaaaaaaaaagagaaa

SEQ ID NO: 73 - DNA, 199 bp, Homo sapiens
tacaaaaaatacaaaagaaaattagctgggcgtgttggcatgggcctgtaacccaactacacggaagctgaggcaggagaatcgcttcaacctgggaggaggaggttgcagtgagctgagatagcatcattgcactccagcctgggcgacaagagtgagacttctctaaagtaaaaataataataataataataaagaa

SEQ ID NO: 74 - DNA, 186 bp, Homo sapiens
ggtggcatgtgtctatagtcccagctacttgggaggctaaagtggaaggattagttgaacccacgaggttgaggcttcagtgaaccatgcactccagcctgagcgacagagcaagacaccattccaagaaagaaaaaaaaaaagactggcaggccaaaaagacagaactgaaattccaaaaaaaaa

SEQ ID NO: 75 - DNA, 188 bp, Homo sapiens
taacatggcgggatgccggtggtgccatgcctgtggtcccagcttcttgggggctgaggtgggaggaccttctgagcctggtggcaaagttgcagtaagctgtgttggtatcactgcactccagcctgcactcctgcacaaagcaagaccctgtctcaaaacaataaataaattaaaatatagagaga

SEQ ID NO: 76 - DNA, 197 bp, Homo sapiens
ctatctctaccaaaaatttaaaaagtagccaagtatgattgaacacacttgtcccagctactcaggaggctgatgggggaggatcacctgagccccggaggccgagggtgtagtgagccatgatcgatgccactgcactccagcctgggccacaaagtgagaccctgtctcaaaaaaaaataataaaaaaaagggaa

SEQ ID NO: 77 - DNA, 192 bp, Homo sapiens
ctggccaacatagtgaaaccccgtctctgctaaaaatacaaaaattcgcgcgcctgtggtcccagctctggggctgaggaatgggaactcacttgaacctgggagatggaggttgccgtgagcggagatcaaaccactgcactccagcctgggcaacagggtgagactgtgtctcaaaaaaaaaaaaaataa

SEQ ID NO: 78 - DNA, 195 bp, Homo sapiens
ctaggcaacagaatgagaccctgtatccacaaaaatatgtagctgggcgtgggggattgtctcaggggactgagacaagaggatcacttgaaccaggaggtccaggctgcagtgagccttgatgccatgaccaggccactgcactccagcctgggcaacacagcaagaccctgtctcaaaaaaaaaaaaaaaaaa

SEQ ID NO: 79 - DNA, 146 bp, Homo sapiens
ttacctgggtgtggtggcgcacctgtagtcccagctacttggaagcagaggtaggaggattacttgaacctcagaggttgaggctgaagtgagccatgattgcaccactgcactccagcctgggcgacaaagtgagaccccaactc

SEQ ID NO: 80 - DNA, 209 bp, Homo sapiens
gtcccctccgcccccgcccccgccccccgccccatctctaggaaaaaatatatatatttttttaattagccgagcgtggtggtgcatacctgcagttccagttactaccctaggtcgaggctgcagcgagccatgatcgcgccactgcactccagcctgggcaccgagcccgttaaaaaaaaaaaaaggaaaagtggcaaaaaccaaaa

SEQ ID NO: 81 - DNA, 202 bp, Homo sapiens
agaccccatctctaaaaattttaaaaattacgtgggtaacgtggcacacatcggcagtcctagctattcaggaggctgagatgggagaattgcttgagagttgcttgagcccaggagttgaggctacagtgagctataatcaagccactgcactccagcctgggtgtcagaaggagaccctgtctttaaaaaaaagaagaaa

SEQ ID NO: 82 - DNA, 228 bp, Homo sapiens
aaaccctgtctctacttaaaatacaaaaaaattagtcaggcgcatgcgggcgcctatagtaccagctactcgggaggctgaggccggagaatggcatgaacccgggaggcggagcttggcagtgagccaagatttgtgccactgcactccagcctgggtgacagagaaagactttgtctcaaaaaaaaaaaaaaagagactcaaagcatacaaggattggaatcagaa

SEQ ID NO: 83 - DNA, 212 bp, Homo sapiens
tacaacaaattaagacctcaaagatcaaaaaattttaaaaattagcagggcgtgtgttgcacacctgtagtctcagagacttggggggctgaggcaggaggatcacttgagcctgggtggtcgaggctgccatgaaccatgatgtgccactgcactccagcctgggatagagcgagatccgagaccctgtctcaaacaaacaaacaaaataa

SEQ ID NO: 84 - DNA, 228 bp, Homo sapiens
ccttgtctctgcaaagtaataataataacaataataataaataaaattagccaggcatggtggtggcatgtgcctgtagtcccaggtattcaagaggttgaggcaggagtctcacttaagcccaagagtttgaggttgctgtgagctatgaatgcactactgcactccagcctgggtgaccgaggaagactcagtaaaaacaaaacaaaacaaacaaacaaacaaaaa

SEQ ID NO: 85 - DNA, 215 bp, Homo sapiens
gtaaaaaaaaaatttttttaattggctgggtgtggtggtggcacatgcctgtgttcccagctactcaggaggctaaggcaggaggatcgcttgagcccagcaggctaaggctgcagtgagctgtcctgctactgcactccagcctgggtgacacagagaccctgtctcaaacaaaacgaaagtaaaccagggagaataaaaaaggaaaaaacaca

SEQ ID NO: 86 - DNA, 217 bp, Homo sapiens
acacagcgagacactgtgtgaaaaaaatgtaaaaattaactgggtgtggtggtgtgcgcctgtagtcccacctgctcagggggctgaggtgggacgatcacttgagcccaagggtttggggtcacgtaacagtaagctatgattgagccactgcactccagcctgggtgacagagagagaccctgtcccaaaaacaaacaacaaacaaacaaaaaaa

SEQ ID NO: 87 - DNA, 228 bp, Homo sapiens
caacatggtgaaagcttcgtctctactaaaaatacaaaaattagctggatgtggtggcatgagcctgtaatcctagctattgaggcaggagaatcgcttgaacccaagaggcagaggttgcagtgagccaagatcacgccactgcactccagcctgggtgacagagccagactccatcttaaaaaaaaaaaaaaggcagagagaaagatacagacaggcaaacacaga

SEQ ID NO: 88 - DNA, 203 bp, Homo sapiens
aacacggtgaaaccccatctccactaaaaaatacaaaaaattagccgggcgtggtggcgggcgcctgtagtcccagctactcaggaggccgaagttgcagtgagccgagatcatgccactgcactccagcctgggtgacagagcgaggctccgtctcaaaaagtaaaaataaaaataaaacaggtaactaatacctaataaag

SEQ ID NO: 89 - DNA, 212 bp, Homo sapiens
aaacccaaaactttcaggtcagacagagtggcataaacctgtaaccccagcattttgggtggctgaggtgggaagatcgcttgggcccaggactttgaggctacagtgagccaatgattatgccactgcactccagcctgggcaacagagcaagactgtctcacacacacacacacacgcacacacaaatgtaaaatgaaactttaaaaaaa

SEQ ID NO: 90 - DNA, 173 bp, Homo sapiens
agagcatgcctatagtcccagctacttgggaggctgaagcttaaggcttgcttgagctctggagttggaggtctgcagtgagccataatcacaccactgcactccagcctgggtgaaagagcaggactctgtctcttaaaaaaaaagaagaagaagaagaagaagaagaagaa

SEQ ID NO: 91 - DNA, 156 bp, Homo sapiens
caagaggcggaggcaggagaatctctttcatgtaggaggcagagactgtagtgagctgaatttgcaccactgcactccagcctgggcaacagagtaaaactccatctcaaaaaacaaaacaaaacaaaaaaaacacataaatagaccaataacaaa

SEQ ID NO: 92 - DNA, 254 bp, Homo sapiens
tgagccacatagcaagatcctcatctctaacaacaacaacaacaaaattaaattaaattagctggatgtggtggcacacacctgtagtcctagctactcagaagggtgagatgggaggctcacttgagccaggaattcaaggttaatagtgtactatgattgagccactgcactccagcctgggcaacaaagcaagaccctgtctctaaaatgaaagaaaaaagaaaggaaggaaagaaggaggaaggaaggag

SEQ ID NO: 93 - DNA, 157 bp, Homo sapiens
ctgcggcaggagaatcgcttgaatccaggaggcggaggttgtggtgagctgagatcacgtcattgcactccagcctgggacaacaagagtgaaactccatctcaaaaaaaaagaaaaaaaagaaagttccctgaaagaaaacacaaagtcaaagaga

SEQ ID NO: 94 - DNA, 175 bp, Homo sapiens
gccagatgcggtggctcacgcctgtaatcccagcactttgggaggctgaggcagttggatcacctgaggtcaggaattagcaccactgcactccagcctaggcgacgagagcaaaactctgtctcaaaaaaaaaaaaaaagaaagaaagaaaaaagaaagggctaggagctacaa

SEQ ID NO: 95 - DNA, 158 bp, Homo sapiens
aagggaggctaagacaggaggaccgcaagagaccaggagttcgaggctgtagtgagccaagactgcaccactgcactccagctcgggtgacagaggtagacctgtctctcaaaaaaaaaaaaaaaaaaaaaagacagaagaaaggcagccactgataa

SEQ ID NO: 96 - DNA, 187 bp, Homo sapiens
ctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggta

SEQ ID NO: 97 - DNA, 143 bp, Homo sapiens
gtggttggcttgagctggggaggcagagagagtgcagtgagctgagatcgcaccactgtactccagcctgggtgataggagccggagggtgtctcaaaaaaaaaaaaagaaagaaaagaaaaagaaaaagaaataagcatcaa

SEQ ID NO: 98 - DNA, 148 bp, Homo sapiens
agggaggcaggaggatcacttgagcccaggagtttgaggcctgcggtgagctgtgatcacaccaccacactccaacctgtgtaacagagtgaggcctcatctcaaaaaaaaaaaaaaggccacaaaactcaacaataaaaacaaacag

SEQ ID NO: 99 - DNA, 19 bp, Homo sapiens
ccagccacagtgctaatgc

SEQ ID NO: 100 - DNA, 20 bp, Homo sapiens
ggccaattttggtattttgg

SEQ ID NO: 101 - DNA, 24 bp, Homo sapiens
actaaaatcaaattagcagcacag

SEQ ID NO: 102 - DNA, 20 bp, Homo sapiens
tagacgggttttgcccttgt

SEQ ID NO: 103 - DNA, 20 bp, Homo sapiens
aaaaattagccagccgtggt

SEQ ID NO: 104 - DNA, 20 bp, Homo sapiens
ttttgagcagtctcgctctg

SEQ ID NO: 105 - DNA, 18 bp, Homo sapiens
ccaggcacagtggctcag

SEQ ID NO: 106 - DNA, 20 bp, Homo sapiens
ttgagacagggtctccttcc

SEQ ID NO: 107 - DNA, 20 bp, Homo sapiens
tcgcttgattccaggatgat

SEQ ID NO: 108 - DNA, 20 bp, Homo sapiens
atctcggctcactgcaaact

SEQ ID NO: 109 - DNA, 20 bp, Homo sapiens
gccaaacatcccactcagaa

SEQ ID NO: 110 - DNA, 20 bp, Homo sapiens
caggatctccatctgtcacc

SEQ ID NO: 111 - DNA, 18 bp, Homo sapiens
ccaggcatggtgctcatc

SEQ ID NO: 112 - DNA, 18 bp, Homo sapiens
attacaggcgcccaccac

SEQ ID NO: 113 - DNA, 20 bp, Homo sapiens
aattaggttgggcacagtgg

SEQ ID NO: 114 - DNA, 20 bp, Homo sapiens
tttcagatggggtcttgctc

SEQ ID NO: 115 - DNA, 20 bp, Homo sapiens
ttgagtagggctgtgtgtgg

SEQ ID NO: 116 - DNA, 23 bp, Homo sapiens
gaagggtctcactctatcatcca

SEQ ID NO: 117 - DNA, 20 bp, Homo sapiens
catggtgaaaccccatctct

SEQ ID NO: 118 - DNA, 20 bp, Homo sapiens
tcctggttccagcaattctc

SEQ ID NO: 119 - DNA, 20 bp, Homo sapiens
gtacttttgggaggccaggt

SEQ ID NO: 120 - DNA, 20 bp, Homo sapiens
cccgaatagctgggattaca

SEQ ID NO: 121 - DNA, 20 bp, Homo sapiens
gagaccagcctgaccaatgt

SEQ ID NO: 122 - DNA, 20 bp, Homo sapiens
acaccgaattcaagcgattc

SEQ ID NO: 123 - DNA, 20 bp, Homo sapiens
atcccagcactttgttaggc

SEQ ID NO: 124 - DNA, 20 bp, Homo sapiens
atctcggctcactgcaaact

SEQ ID NO: 125 - DNA, 20 bp, Homo sapiens
tgtgatggctcacacctgta

SEQ ID NO: 126 - DNA, 20 bp, Homo sapiens
ctcactgcaacctccacctc

SEQ ID NO: 127 - DNA, 18 bp, Homo sapiens
tgattcttggccgggtgt

SEQ ID NO: 128 - DNA, 19 bp, Homo sapiens
gggctcaagcgattctcct

SEQ ID NO: 129 - DNA, 20 bp, Homo sapiens
tggcaaaaccccatctctac

SEQ ID NO: 130 - DNA, 20 bp, Homo sapiens
gtcttgctctgtcgctaggc

SEQ ID NO: 131 - DNA, 20 bp, Homo sapiens
ggtgaaacccaccagtctct

SEQ ID NO: 132 - DNA, 20 bp, Homo sapiens
ctgacggagttccactcttg

SEQ ID NO: 133 - DNA, 21 bp, Homo sapiens
aacatggtgaaaccctgtgtc

SEQ ID NO: 134 - DNA, 20 bp, Homo sapiens
tgcaatgacgtgatctcagc

SEQ ID NO: 135 - DNA, 25 bp, Homo sapiens
ccccgtctctactaaaaatacaaaa

SEQ ID NO: 136 - DNA, 20 bp, Homo sapiens
tattacccaggctggagtgc

SEQ ID NO: 137 - DNA, 24 bp, Homo sapiens
ccaacatggtgtaaacactgtctc

SEQ ID NO: 138 - DNA, 20 bp, Homo sapiens
gatgacaggctcactgcaac

SEQ ID NO: 139 - DNA, 20 bp, Homo sapiens
catgatgaaaccccacctct

SEQ ID NO: 140 - DNA, 20 bp, Homo sapiens
ctctgtcacccaggctgaat

SEQ ID NO: 141 - DNA, 20 bp, Homo sapiens
aacacggagaaaccccttct

SEQ ID NO: 142 - DNA, 20 bp, Homo sapiens
tgcaatgacgtgatctcagc

SEQ ID NO: 143 - DNA, 20 bp, Homo sapiens
cggtgactcacgcctgtaat

SEQ ID NO: 144 - DNA, 19 bp, Homo sapiens
taccagcatacgccacgac

SEQ ID NO: 145 - DNA, 20 bp, Homo sapiens
tcgcttgattccaggatgat

SEQ ID NO: 146 - DNA, 20 bp, Homo sapiens
ggctcactgcaaacttccac

SEQ ID NO: 147 - DNA, 19 bp, Homo sapiens
cggttcacgcctgtaattc

SEQ ID NO: 148 - DNA, 22 bp, Homo sapiens
ctcccaagtagctgtgactaca

SEQ ID NO: 149 - DNA, 18 bp, Homo sapiens
ccaggcatggtgctcatc

SEQ ID NO: 150 - DNA, 18 bp, Homo sapiens
attacaggcgcccaccac

SEQ ID NO: 151 - DNA, 20 bp, Homo sapiens
atctcagcactttgggaagc

SEQ ID NO: 152 - DNA, 20 bp, Homo sapiens
ctcccgagtagctgggatta

SEQ ID NO: 153 - DNA, 20 bp, Homo sapiens
aatcccagcactttcagtgg

SEQ ID NO: 154 - DNA, 20 bp, Homo sapiens
ccatgtctggctacggttct

SEQ ID NO: 155 - DNA, 20 bp, Homo sapiens
cagcactttgggaggctaga

SEQ ID NO: 156 - DNA, 18 bp, Homo sapiens
cgggtagcgggattacag

SEQ ID NO: 157 - DNA, 20 bp, Homo sapiens
gtacttttgggaggccaggt

SEQ ID NO: 158 - DNA, 20 bp, Homo sapiens
cccgaatagctgggattaca

SEQ ID NO: 159 - DNA, 20 bp, Homo sapiens
cacgaggtcaggacatcaga

SEQ ID NO: 160 - DNA, 19 bp, Homo sapiens
atctggctcacggcaagtc

SEQ ID NO: 161 - DNA, 20 bp, Homo sapiens
cccgtaatcccagcacttag

SEQ ID NO: 162 - DNA, 20 bp, Homo sapiens
gttccagcaattctgcctca

SEQ ID NO: 163 - DNA, 20 bp, Homo sapiens
tggtggctcacacctgtaat

SEQ ID NO: 164 - DNA, 20 bp, Homo sapiens
acgatctcagctcactgcac

SEQ ID NO: 165 - DNA, 18 bp, Homo sapiens
gcagtgctcacgcctcac

SEQ ID NO: 166 - DNA, 20 bp, Homo sapiens
tgattacaggcgtccactga

SEQ ID NO: 167 - DNA, 19 bp, Homo sapiens
gggtgcatcacctgacatc

SEQ ID NO: 168 - DNA, 20 bp, Homo sapiens
attctcctgcctcagcttcc

SEQ ID NO: 169 - DNA, 20 bp, Homo sapiens
agtggctcatgcctgaaatc

SEQ ID NO: 170 - DNA, 18 bp, Homo sapiens
attcctcagccccagagc

SEQ ID NO: 171 - DNA, 20 bp, Homo sapiens
tcgtgtgtggtggctcatac

SEQ ID NO: 172 - DNA, 20 bp, Homo sapiens
ccaccatccccagctaattt

SEQ ID NO: 173 - DNA, 19 bp, Homo sapiens
ctgtggtctcggcactttg

SEQ ID NO: 174 - DNA, 20 bp, homo sapiens
tctcctgcctcagtctcctg

SEQ ID NO: 175 - DNA, 20 bp, Homo sapiens
cctagcactttgggagctga

SEQ ID NO: 176 - DNA, 20 bp, Homo sapiens
caccacaccccagctaattt

SEQ ID NO: 177 - DNA, 19 bp, Homo sapiens
cagcactttgggaggtcac

SEQ ID NO: 178 - DNA, 20 bp, Homo sapiens
attacaggcatgtgccacca

SEQ ID NO: 179 - DNA, 20 bp, Homo sapiens
tgcagtggcttatgcctgta

SEQ ID NO: 180 - DNA, 18 bp, Homo sapiens
aggtgcatgccaccacac

SEQ ID NO: 181 - DNA, 20 bp, Homo sapiens
cagtggctcaggcctgtaat

SEQ ID NO: 182 - DNA, 21 bp, Homo sapiens
tcctgcctcaatagctaggat

SEQ ID NO: 183 - DNA, 20 bp, Homo sapiens
gtgcagtctcacgcctgtaa

SEQ ID NO: 184 - DNA, 20 bp, Homo sapiens
tgccaccacacttggctaat

SEQ ID NO: 185 - DNA, 20 bp, Homo sapiens
ctagctactcgggaggctga

SEQ ID NO: 186 - DNA, 19 bp, Homo sapiens
acggagctcggtctattgc

SEQ ID NO: 187 - DNA, 20 bp, Homo sapiens
tcatgcctataatcccagca

SEQ ID NO: 188 - DNA, 24 bp, homo sapiens
tttttgtttttgcttttgttttga

SEQ ID NO: 189 - DNA, 20 bp, Homo sapiens
agacaagccaggtgaggtgt

SEQ ID NO: 190 - DNA, 22 bp, Homo sapiens
gacagggtctcactctctttcc

SEQ ID NO: 191 - DNA, 24 bp, Homo sapiens
actaaaatcaaattagcagcacag

SEQ ID NO: 192 - DNA, 20 bp, Homo sapiens
tagacgggttttgcccttgt

SEQ ID NO: 193 - DNA, 21 bp, Homo sapiens
ttagccaggcatagttgtgtg

SEQ ID NO: 194 - DNA, 20 bp, Homo sapiens
ttttggacacagggtcttgc

SEQ ID NO: 195 - DNA, 20 bp, Homo sapiens
gctgaggcaggagaatcact

SEQ ID NO: 196 - DNA, 27 bp, Homo sapiens
ttttatgctattataaatggaattgct

SEQ ID NO: 197 - DNA, 20 bp, Homo sapiens
catggtgaaaccccatctct

SEQ ID NO: 198 - DNA, 20 bp, Homo sapiens
gacacagcctcactctgtcg

SEQ ID NO: 199 - DNA, 23 bp, Homo sapiens
aataatataagctgggcttgtgg

SEQ ID NO: 200 - DNA, 20 bp, Homo sapiens
tttttggaaacagggtctca

SEQ ID NO: 201 - DNA, 20 bp, Homo sapiens
cgcacctgtaatcccagcta

SEQ ID NO: 202 - DNA, 20 bp, Homo sapiens
ttctttccctttccctttcc

SEQ ID NO: 203 - DNA, 22 bp, Homo sapiens
ttataaactggagctgggtgtg

SEQ ID NO: 204 - DNA, 21 bp, Homo sapiens
gatagggtcttgctctgttgc

SEQ ID NO: 205 - DNA, 20 bp, Homo sapiens
gaaggctgagatggatggac

SEQ ID NO: 206 - DNA, 24 bp, Homo sapiens
tcttgcttctctctctctctctcc

SEQ ID NO: 207 - DNA, 20 bp, Homo sapiens
gatccagccacatgtgaacc

SEQ ID NO: 208 - DNA, 20 bp, Homo sapiens
tccaaatggagtttcgctct

SEQ ID NO: 209 - DNA, 20 bp, Homo sapiens
cggaagtgggaggatttctt

SEQ ID NO: 210 - DNA, 23 bp, Homo sapiens
gagacaggatctctctcttttgc

SEQ ID NO: 211 - DNA, 20 bp, Homo sapiens
gaaaccccgtgtctaccaaa

SEQ ID NO: 212 - DNA, 22 bp, Homo sapiens
tctgagatggagtcttgctctg

SEQ ID NO: 213 - DNA, 20 bp, Homo sapiens
ccagcttgaacaacacagca

SEQ ID NO: 214 - DNA, 20 bp, Homo sapiens
caaggtcttgctctgtcacc

SEQ ID NO: 215 - DNA, 18 bp, Homo sapiens
gtggcgcatgcctgtaac

SEQ ID NO: 216 - DNA, 27 bp, Homo sapiens
ttatttatttctgagacagagtcttgc

SEQ ID NO: 217 - DNA, 18 bp, Homo sapiens
gtgggacaggcccgtagt

SEQ ID NO: 218 - DNA, 25 bp, Homo sapiens
tttatttttacagacagggtcttgg

SEQ ID NO: 219 - DNA, 18 bp, Homo sapiens
aaatggcccagcttggtg

SEQ ID NO: 220 - DNA, 25 bp, Homo sapiens
tttcttgtcgcttcctattttattt

SEQ ID NO: 221 - DNA, 23 bp, Homo sapiens
tgaaaccccgtctctactaaaaa

SEQ ID NO: 222 - DNA, 25 bp, Homo sapiens
tttagacagagtctcactgttttgc

SEQ ID NO: 223 - DNA, 20 bp, Homo sapiens
aaaattagctgggcgtgttg

SEQ ID NO: 224 - DNA, 24 bp, Homo sapiens
tttagagaagtctcactcttgtcg

SEQ ID NO: 225 - DNA, 20 bp, Homo sapiens
ccagctacttgggaggctaa

SEQ ID NO: 226 - DNA, 24 bp, Homo sapiens
tggaatttcagttctgtctttttg

SEQ ID NO: 227 - DNA, 19 bp, Homo sapiens
cctgtggtcccagcttctt

SEQ ID NO: 228 - DNA, 20 bp, Homo sapiens
tcttgctttgtgcaggagtg

SEQ ID NO: 229 - DNA, 24 bp, Homo sapiens
ccaaaaatttaaaaagtagccaag

SEQ ID NO: 230 - DNA, 21 bp, Homo sapiens
agacagggtctcactttgtgg

SEQ ID NO: 231 - DNA, 20 bp, Homo sapiens
gtgaaaccccgtctctgcta

SEQ ID NO: 232 - DNA, 20 bp, Homo sapiens
acacagtctcaccctgttgc

SEQ ID NO: 233 - DNA, 22 bp, Homo sapiens
gaatgagaccctgtatccacaa

SEQ ID NO: 234 - DNA, 19 bp, Homo sapiens
cagggtcttgctgtgttgc

SEQ ID NO: 235 - DNA, 20 bp, Homo sapiens
tggaagcagaggtaggagga

SEQ ID NO: 236 - DNA, 20 bp, Homo sapiens
gttggggtctcactttgtcg

SEQ ID NO: 237 - DNA, 21 bp, Homo sapiens
ttttaattagccgagcgtggt

SEQ ID NO: 238 - DNA, 21 bp, Homo sapiens
ttttggtttttgccacttttc

SEQ ID NO: 239 - DNA, 19 bp, Homo sapiens
gggtaacgtggcacacatc

SEQ ID NO: 240 - DNA, 21 bp, Homo sapiens
acagggtctccttctgacacc

SEQ ID NO: 241 - DNA, 19 bp, Homo sapiens
agctactcgggaggctgag

SEQ ID NO: 242 - DNA, 22 bp, Homo sapiens
ttccaatccttgtatgctttga

SEQ ID NO: 243 - DNA, 20 bp, Homo sapiens
aaaaattagcagggcgtgtg

SEQ ID NO: 244 - DNA, 20 bp, Homo sapiens
tgagacagggtctcggatct

SEQ ID NO: 245 - DNA, 20 bp, Homo sapiens
aaaattagccaggcatggtg

SEQ ID NO: 246 - DNA, 22 bp, Homo sapiens
tttactgagtcttcctcggtca

SEQ ID NO: 247 - DNA, 20 bp, Homo sapiens
tttaattggctgggtgtggt

SEQ ID NO: 248 - DNA, 21 bp, Homo sapiens
agacagggtctctgtgtcacc

SEQ ID NO: 249 - DNA, 20 bp, Homo sapiens
taactgggtgtggtggtgtg

SEQ ID NO: 250 - DNA, 20 bp, Homo sapiens
tgtttttgggacagggtctc

SEQ ID NO: 251 - DNA, 20 bp, Homo sapiens
caacatggtgaaagcttcgt

SEQ ID NO: 252 - DNA, 21 bp, Homo sapiens
agatggagtctggctctgtca

SEQ ID NO: 253 - DNA, 19 bp, Homo sapiens
acacggtgaaaccccatct

SEQ ID NO: 254 - DNA, 18 bp, Homo sapiens
gagcctcgctctgtcacc

SEQ ID NO: 255 - DNA, 20 bp, Homo sapiens
cctgtaaccccagcattttg

SEQ ID NO: 256 - DNA, 20 bp, Homo sapiens
catttgtgtgtgcgtgtgtg

SEQ ID NO: 257 - DNA, 20 bp, Homo sapiens
cagctacttgggaggctgaa

SEQ ID NO: 258 - DNA, 27 bp, Homo sapiens
ttcttcttcttcttcttcttcttcttc

SEQ ID NO: 259 - DNA, 22 bp, Homo sapiens
ggcaggagaatctctttcatgt

SEQ ID NO: 260 - DNA, 25 bp, Homo sapiens
tttgagatggagttttactctgttg

SEQ ID NO: 261 - DNA, 20 bp, Homo sapiens
gtggtggcacacacctgtag

SEQ ID NO: 262 - DNA, 21 bp, Homo sapiens
ccttcctccttctttccttcc

SEQ ID NO: 263 - DNA, 20 bp, Homo sapiens
gcaggagaatcgcttgaatc

SEQ ID NO: 264 - DNA, 24 bp, Homo sapiens
tctttgactttgtgttttctttca

SEQ ID NO: 265 - DNA, 20 bp, Homo sapiens
gcctgtaatcccagcacttt

SEQ ID NO: 266 - DNA, 23 bp, Homo sapiens
tgtagctcctagccctttctttt

SEQ ID NO: 267 - DNA, 20 bp, Homo sapiens
gggaggctaagacaggagga

SEQ ID NO: 268 - DNA, 20 bp, Homo sapiens
tcagtggctgcctttcttct

SEQ ID NO: 269 - DNA, 20 bp, homo sapiens
gtggcgcacacctatagtcc

SEQ ID NO: 270 - DNA, 25 bp, Homo sapiens
cctttctcttcacaatttgtctctt

SEQ ID NO: 271 - DNA, 20 bp, Homo sapiens
agtgcagtgagctgagatcg

SEQ ID NO: 272 - DNA, 27 bp, Homo sapiens
tgcttatttctttttctttttcttttc

SEQ ID NO: 273 - DNA, 20 bp, Homo sapiens
cttgagcccaggagtttgag

SEQ ID NO: 274 - DNA, 21 bp, Homo sapiens
tgttgagttttgtggcctttt

SEQ ID NO: 275 - DNA, 325 bp, Unknown
  Species of African Cichlid
gggcgatcgtggctcaagagttgggagttcgccttgtaaycggaaggttgccggttcgagccccggctcggacagtctcggtcgttgtgtccttgggcaagacacttcacccgttgcctactggtggtggccagaggggccgatggcgcgatatggcagcctcgcctctgtcagtsygccccagggyrgctgtggctacaactgtagcttgcctycaccagtgtgtgaatgtgtgagtgaatgagtggaattgtaaagcgctttggggtccttagggaccagaaaagcgctatataaatacagtccatttayyattattattatt

SEQ ID NO: 276 - DNA, 289 bp, Unknown
  Species of African cichlid
ggggtggctgtagctcaggaggtagagcaggtcatctactaatcggaaggttggtggttcgatccctggctgctccagtctgcatgccaaatatccttgggcaagatactaaccccaagttgctctccgatgcatccatcggagtgtgaatgtgtgtgaatgttagatagaaagcacttagaaaaaagtgcttgtgtgaatgggtgaatgaggcatgttgtataaagcgctttgagtgctcaggkagagtagaaaagcgctatataagaaccagtccatttaccattta

SEQ ID NO: 277 - DNA, 230 bp, Unknown
  Species of African cichlid
ggggcagggatagctcagtaggtagagtggtggccccatgatcggaaggtcgggggttcgaatccactgaacggctaccctgaggtacccctgagcaaggtaccgtccctacacactgctccccgggcgccgcattggtggctgcccactgcttcactgagtgaatgggttaaatgcagagaagkaatttccccacggggatcaataaagtatacattattattattatt

SEQ ID NO: 278 - DNA, 168 bp, Unknown
  Proboscidea
ggaaaccctggtggcgtagtggttaagagctacggctgctaaccaaaaggtcagcagttcgaatccaccaggcgctccttggaaaccctatggggcagttctactctgtcctatagggtcgctatgagtcggaatcgactcgacggcaatgggtttggtttttttttt

SEQ ID NO: 279 - DNA, 222 bp, Unknown
  afrotherians
ggaaaccctggtggcatagtggttaagtgctatggctgctaaccaaaaggtcggcagttcraatccaccagctgctccgcgggagaaagatgtggcagtctgcttccgtaaagatttacagccttggaaaccctatggggcagttctactctgtcctatagggtcactatgagtcggaattgactccatggcagtgggctttggttttgtttattcgtttct

SEQ ID NO: 280 - DNA, 290 bp, Homo sapiens
ggccgggcgcggtggctcacgcctgtaatcccagcactttgggaggccgaggcgggcggatcacgaggtcaggagatcgagaccatcctggccaacacggtgaaaccccgtctctactaaaaatacaaaaattagccgggcgtggtggcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaa

SEQ ID NO: 281 - DNA, 145 bp, Mus musculus
  misc_feature (89)..(89): n is a, c, g, or t
gccgggcatggtggcgcacgcctttaatcccagcacttgggaggcagaggcaggcggatttctgagttcgaggccagcctggtctacanagtgagttccaggacagccagggctacacagagaaaccctgtctcgaaaaaaaaaa

SEQ ID NO: 282 - DNA, 221 bp, Unknown
  Gliridae, Sciuridae & Aplodontidae
agccgggtgtggtggcgcaygcctgtaatcccagcsacttgggaggctgaggcaggaggatcacaagttcaaggccagcctcagcaacttagtgaggccctaagcaacttagtgagaccctgtctcaaaataaaaaaaaaaaaaaaaggggctggggatgtggctcagtggtagagtgcccctgggttcaatccccagtaccaaaaaaaaaaaaaaaaaaa

SEQ ID NO: 283 - DNA, 299 bp, Mus musculus
gggctggggaggatggctcagtcggtaaagtgcttgctgtgcaagcatgaggacctgagttcagatccccagaacccacatataaaaaagccaggcatggtggtatgtgcttgtaatcccagygctggggaggcagagacaggaggatccctggggctcgctggccagccagcctagcctaattggtgagctccaggttcagtgagagaccctgtctcaaaaaataaggtggagagtaactgaggaagacacctgaggttgacctctggcctccacatacacacacacacacacacaca

SEQ ID NO: 284 - DNA, 329 bp, Unknown
  Bovidae
gggcttccctggtagctcagctggtaaagaatctgcctgcaatgcaggagaccctggttcgattcctgggtcgggaagatcccctggagaagggataggctacccactccagtattcttgggcttccctggtggctcagctggtaaagaatctgcctgcaatgcgggagacctgggttcgatccctgggttgggaagatcccctggagaagggaatggctacccactccagtattctggcctggagaattccatggacagaagagtccatggggtcgcaaagagtcggacacgactgagcgactttcactttcacttactttataaaat

SEQ ID NO: 285 - DNA, 210 bp, Unknown
  Bovidae
gggcttccctggtggctcagatggtaaagaatctgcctgcaatgcgggagacctgggttcratccctgggttgggaagatcccctggagaagggcatggcaacccactccagtattcttgcctggagaatcccatggacagaggagcctggtgggctacagtccatggggtcgcaaagagtcggacacgactgagcgactaagcacacac

SEQ ID NO: 286 - DNA, 335 bp, Unknown
  Lagomorpha
ggggccggcgctgtggcgcagtaggttaagcctccgcctgcagtgccggcatcccatatgggcgccggttcgagtcccggctgctccacttccaatccagctctctgctaatggcctgggaaagcagtrgaagatggcccaagtgcttgggcccctgcacccacgtgggagacccggaagaagctcctggctcctggcttcggatcggcccagctccggccattgtggccatttggggagtgaaccagcggatggaagacctctctctctctctctctctctctctctctgtaactctgcctttcaaataaataaataaawttttaaaaaaaaaa

SEQ ID NO: 287 - DNA, 175 bp, Unknown
  Carnivora
ggggcgcctgggtggctcagtcggttaagcgtctgccttcggctcaggtcatgatctcagggtcctgggatcgagccccgcatcgggctccctgctcagcrgggagcctgctttctccctctctctctctctctctctctctctctctctctctctctcttctcaaaaaaaaaaa

SEQ ID NO: 288 - DNA, 119 bp, Unknown
  cetaceans, hippopotamuses, ruminants & suiforms
gggacttccctggtggtccagtggttaggactccgcgctttcactgcagggggcccgggttcaatccctggtcggggaactaagatcccacatgccgygtggcacagccaaaaaaaaaa

SEQ ID NO: 289 - DNA, 321 bp, Unknown
  cetaceans, hippopotamuses, ruminants & suiforms
gggcttccctggtggcgcagtggttaagaatccgcctgccaatgcaggggacacgggttcgagccctggtccgggaagatcccacatgccgcggagcaactaagcccgtgcgccacaactactgagcctgcgctctagagcccgcgagccacaactactgagcccacgtgccacaactactgaagcccgcgcgcctagagcccgtgctccgcaacaagagaagccaccgcaatgagaagcccgcgcaccgcaacgaagagtagcccccgctcgccgcaactagagaaagcccgcgcgcagcaacgaagacccaacacagcc

SEQ ID NO: 290 - DNA, 407 bp, Equus caballus
agccagccctggtggtctagtggttaagattcggcgctctcactgccgcggcctgggtttgtttcccggtcagggaaccacaccacccgtctgtcggttgtcatactgtggcagctgcgtgttgctgtgatgctgaaagctatgccaccagtatttcaaataccagcagggtcacccatggtggacaggtttcagcggagcttccagactaagacagactaggaagaaggacctggccacccacttccaaaaaaattggccatgaaaaccctatgaatagcagtggagcattgtctgatacagcgccagaaggtgagaggatggcgcaaaaagaccgggcagggttccgctctgctgtacacagggtcgctaggagtcagaatcgactcgatggcactaacaacaaa

SEQ ID NO: 291 - DNA, 261 bp, Equus caballus
ggggccggccccgtggccgagtggttaagttcgcgcgctccgcttcggcggcccagggtttcgctggttcggatcctgggcgcggacatggcaccgctcatcaggccatgctgaggcggcgtcccacatagcacaactagaaggacctacaactagaatatacaactatgtactggggggctttggggagaaaaagaaaaaaaaaaaaaaaagaagattggcaacagatgttagctcaggtgccaatctttaaaaaaaaaa

SEQ ID NO: 292 - DNA, 249 bp, Equus caballus
ggggccagccccgtggcctagtggttaagttcggcgcgctccgcttcggcggcccggggttcgcmggttcggatcccgggcgcggacctacaccactcrtcaggccatgctgtggcggcgtcccacatacaaaatagaggaagattggcacagatgttagctcagggcyaatcttcctcaagaaaaaagaggaagattggcaacagatgttagctcagggcgaatcttcctcagcaaaaaaaaaaaaaa

SEQ ID NO: 293 - DNA, 249 bp, Equus caballus; identical to SEQ ID NO: 292

SEQ ID NO: 294 - DNA, 115 bp, Unknown
  Carnivora
ggggcgcctggctggctcagtcggtagagcatgcgactcttgatctcggggttgtgagttcgagccccacgttgggtgtagagattacttaaaaataaaatctttaaaaaaaaaa

SEQ ID NO: 295 - DNA, 85 bp, Mus musculus
ggggttggggatttagctcagtggtagagcgcttgcctagcaagcgcaaggccctgggttcggtcctcagctccgaaaaaaaaaa

SEQ ID NO: 296 - DNA, 481 bp, Unknown
  Sarcopterygii
ggggactggatggctcagtggaattggtaatgggatatggagcctttcacctctaggtcactgggttcaaatccagcccaggtcagtagtgaccgaaagtcattaccatctgatggctgttcagtggcctatgtgaaatgagttggtggtctcagtccagttcctagtggacaggtgtccacatcacaaaaccaccatcacaattggcactaattggcacccttgttggcagtctcagcagagaggccaaggattgaatgggcatggagactgaactaccctctcaaccctgtagaggtggtccctccagggcagggttgaggcacattggcagggcaatgtggggaagcctgcactgctgctgcccatgctgtacctgttctgtggataaatagaggacttcagtctctggtgctatcaatctagcacctttcacgagcactaaattcacacaaaaaaatttaaaaaaaaaaaaaaaaaa

SEQ ID NO: 297 - DNA, 322 bp, Unknown
  Macropodoidea
gggcagctaggtggtgcagtggatagagcaccagtgcaggagtcaggaggacctgagttcaaatctcacctcagacacttgacactcactagctgtgtgaccttgggcaagtcacttaaccccaattgcctcatcctgggtcatctccagtcatcctgatgaatatctggtcactggattcagatggctctggaggagaagtgaggctggtgacctgcacagccctccctcactcaaaacaaagtcaagtgcaagtcatgtcattatttctctgatggcatggcatggtcttcttyggcaaygaaggacgaacacacacaca

SEQ ID NO: 298 - DNA, 298 bp, Ornithorhynchus anatinus
gtctgtggctggttagctcagttggttagagcacggtgctaatgaggccaaggtcacrggtttgatcccygtgtgggccagttagctttgctctgttccatggccacagactgcacccctaaccccagccagctgtctygcaaatgtgtgctgttggtcacaagggggactgggbaagagagtgtggatggatcagtgcaaatccatcaccactactggaaaaacaactcaaagcacatgtcctactgatagtgggtcagtagtgtcatcttcgtatacgaaggacagcacaatcaya

SEQ ID NO: 299 - DNA, 258 bp, Unknown
  Metatheria
ggggcagctaggtggcgcagtggatagagtactaggcctggagtcaggaagactcatcttcctgagttcaaatctggcctcagacacttactagctgtgtgaccctgggcaagtcacttaaccctgtttgcctcagtttcctcatctgtaaaatgagctggagaaggaaatggcaaaccactccagtatctttgccaagaaaaccccaaatggggtcacaaagagtcagacatgactgaaaaaactcaacaacaacaa

SEQ ID NO: 300 - DNA, 139 bp, Unknown
  Metatheria
aggggcagctaggtggcacagtggatagagcactggccctggagtcaggaggacctgagttcaaatctggcctcagacacttactagctgtgtgaccctgggcaagtcacttaaccccaattgcctcaaaaaaaaaaaa

SEQ ID NO: 301 - DNA, 182 bp, Homo sapiens
gctcagttggttagagcatagtgctaatgaggccaaggtcatgggttcaatccccatatgggccagttagcttcacacagagaaaahattgtgttccatggccatagactgcacccctaaccctagccagctgtctcamaaatgtatgccactggtcacaaggggaactrggaaaaaaaaaa

SEQ ID NO: 302 - DNA, 269 bp, Unknown
  Menetes & Callosciurus
ggggctggagawgtggctcagccggtagagcgcttgccttgcaagcgtgagkccstgggttcdattcccagcaccacatgcawatcaatgcctgaaagaaagctcatcaaccactaatatacataaaaatgagctgggcgtggtggcgcacgcctgtaatcccagcagctygggaggctgagactggaggatcrcgagttcaaagccacctcagcaraaataagcgaggtgctaaagcagctcagtgagaccctgtctcaaaaaaaaaa

SEQ ID NO: 303 - DNA, 272 bp, Unknown
  monotremes
agagaagcagcgtggcctagtggatagagcacgggcctgggagtcagaaggacctgggttctaatcccggctccgccacttgtctgctgtgtgaccttgggcaagtcacttmacttctctgtgcctcagttacctcatctgtaaaatggggattaagactgtgagccccacgtgggacagggactgtgtccaacctgattagcttgtactaccccagcgcttagaacagtgcctggcacatagtaagcgcttaacaaataccataattatta

SEQ ID NO: 304 - DNA, 342 bp, Procavia capensis
tggtggttcagtggtagaatttctcgccttccatgcgggagacccaggttcgattcccggccartgcacctcatgcgcagccaccacccatctgtcagtggaggcttgcgtgttgctatgatgctgaacaggtttcagcggagcttccagactaagacggactaggaagaaaggcctggcgatctacttccgaaaatcagccaatgaaaacccctatggatcacaatggtctgatctgcaaccgatcatggggatggcgcaggaccgggcagcatttcgttccattgtgcatggggtcaccatgagttaggggccaactcgacggcagctaacaacaacaac

SEQ ID NO: 305 - DNA, 243 bp, Pedetes capensis
gccctgtggcatccgatcccgtcagatctcggaagctaagcagggtcgggcctggttagtacttggatgggagaccgcctgggaacaccagatggagaaggacctggcaaccccctcccaaaaatcaatctgccattgaaaaccctatgaacagcagagaagcactgacaatagtgtttcccctgttgtacatagggtcgctatgagtcggataccgactcgacggcacctaacaacaacaac

SEQ ID NO: 306 - DNA, 197 bp, Pedetes capensis
gggctgggatttagctcagttggtagagcgctgcgctgcagtctctgaggtcccgagttcaattcccagcaccgcaaaaatgggagaaggacctggcaatccactcccaaggcaatcccgaaacagtcagccattgaaaaccctatgagggtcgctatgagtcggataccgactcgacggcacctaacaacaacaac

SEQ ID NO: 307 - DNA, 237 bp, Unknown
  Species of Picea
gtcaattgggtstagctcgagtggttgtgaggaawgattgttatcctccccaccyaggttcaattcccacwagtgacatgttgagctcttaatgagtatattgttgtaatggcttcgagccttgttgttgacctggccttgtgccctgaagawtctagggtttaccctgggtaccatcctcgaaatgaggggaagtacctagggggtttccccgggatttgtwacctaaaaaaaaaa

SEQ ID NO: 308 - DNA, 243 bp, Sus scrofa
  misc_feature (77)..(77): n is a, c, g, or t
  misc_feature (97)..(97): n is a, c, g, or t
  misc_feature (111)..(111): n is a, c, g, or t
  misc_feature (125)..(125): n is a, c, g, or t
  misc_feature (190)..(190): n is a, c, g, or t
  misc_feature (227)..(227): n is a, c, g, or t
agggagttcccatcgtggctcagtggtamcgaacctgactagtatccatgaggatgcagattcaatccctggccttnctcagtcggttaaggatccngcattgctgtgagntgtggtgtaggtcncagatgcggctcggatcccgtgttgctgtggctgtggtgtaggccggcagctacagctccgattngacccctagcctgggaacctccatatgccgcgggtgnggccctaaaaaaaaaa

SEQ ID NO: 309 - DNA, 182 bp, Unknown
  Species of Rhinolophidae or Hipposideridae
ggggyggccggttrgctcagttggttagagtgcagtgctcataacaccaaggtygccggttcgattcccacatgggccagtgagctgtgccctccacaactagattgaaaacaacracttgacttggagctgatgggtcctggaaaaaccactgttccccaatattccccatttttaaawaa

SEQ ID NO: 310 - DNA, 149 bp, Mus musculus
ggggctggcgagatggctcagtgggtaaaggtgcttgccgccaagcctgatgacctgagttcgatccccgggacccacatggtggaaggagagaaccgactcctacagttgtcctctgacctccacacatgyaccgtggcacacacaca

SEQ ID NO: 311 - DNA, 131 bp, Sus scrofa
ggagttccctggtggctcagygggttaaggatctagtgttgtcactgctgtggctcrggtcactgctgtggtgcaggtttgatccctggcctgggaacttccacatgcygyrggcatggccaaaaaaaaaa

SEQ ID NO: 312 - DNA, 227 bp, Spermophilus tridecemlineatus
gggctggggttgtggctcagtggtagagcgcttgcctagcatgtgtgaggcactgggttcgatcctcagcaccacataaaaataaaataaataaaaagggctggggatgtggctcaagcggtagcgcgctcgcctggcatgcgtgcggcctgggttcgatcctcagcaccacatacaaacaaagatgttgtgtccaccgaaaactaaaaaataaatattaaaaaaaa

SEQ ID NO: 313 - DNA, 263 bp, Unknown
  Species of Talpidae
gggcctccctggtggcgcagtggttgagcrctgggctgcgaagctgcccrcagcctctgcagcaccggttcgatccccggccaccgggcgagggtcccacatggcgcagcagacctctcctgccgcccgctgctcccctcagcagtgtatttcatcawgtctgcctgttctgytccccgctctggctctggtgaattctctcacccccccccyctyykgmctgtacccagtkcttgtataaataaataaatatatctttaaaa

SEQ ID NO: 314 - DNA, 263 bp, Homo sapiens
gcwgkgtggcgcagtggatagagcacgggacctggagtcaggaagacctgggttcgaatcccggctctgccacttactagctgtgtgaccttgggcaagtcacttaacctctctgtgcctcagtttcctcatctgtaaaatggggataataatagcacctacctcacagggttgttgtgaggattaaatgagataatatatgtaaagcgcttagaacagtgcctggcacatagtaagcgctatataaatgttagctattatta

SEQ ID NO: 315 - DNA, 215 bp, Homo sapiens
grgaagcagygtggtacagtggaaagagcactggatttggagtcaggaggacctgggttcaaatcccggctctgacacttactagctgtgtgaccttgggcaagtcacttaacctctctgggcctcagtttcctcatctgtaaaatgagggggttggactagatggcctctaaggtcccttccagctctaaatctatgatcctatgatcctatga

SEQ ID NO: 316 - DNA, 245 bp, Macropus eugenii
gagaggcagtatggcatagtggatagagagctggccttaaagccaggaagacctgggttcaagtcctgcctctgacacatactggctgtgtgaccctgggcaagtcacttaacctctcagtgctctaggcaactctctaagactataagttgcagagaaggtgctaacctgcattggtagagggagtttcctcatctgggagttccctataccaatgaaatcacaggtccagtccctatccctat

SEQ ID NO: 317 - DNA, 14 bp, Homo sapiens
tttttttttttttt

SEQ ID NO: 318 - DNA, 14 bp, Homo sapiens
aaaaaaaaaaaaaa

SEQ ID NO: 319 - DNA, 14 bp, Homo sapiens
gtgtgtgtgtgtgt

SEQ ID NO: 320 - DNA, 14 bp, Homo sapiens
acacacacacacac

SEQ ID NO: 321 - DNA, 14 bp, Homo sapiens
tgtgtgtgtgtgtg

SEQ ID NO: 322 - DNA, 14 bp, Homo sapiens
cacacacacacaca

SEQ ID NO: 323 - DNA, 14 bp, Homo sapiens
gctgggattacagg

SEQ ID NO: 324 - DNA, 14 bp, Homo sapiens
cctgtaatcccagc

SEQ ID NO: 325 - DNA, 14 bp, Homo sapiens
ctgggattacaggc

SEQ ID NO: 326 - DNA, 14 bp, Homo sapiens
gcctgtaatcccag

SEQ ID NO: 327 - DNA, 14 bp, Homo sapiens
agcactttgggagg

SEQ ID NO: 328 - DNA, 14 bp, Homo sapiens
cctcccaaagtgct

SEQ ID NO: 329 - DNA, 14 bp, Homo sapiens
aagtgctgggatta

SEQ ID NO: 330 - DNA, 14 bp, Homo sapiens
aaagtgctgggatt

SEQ ID NO: 331 - DNA, 14 bp, Homo sapiens
taatcccagcactt

SEQ ID NO: 332 - DNA, 14 bp, Homo sapiens
gcactttgggaggc

SEQ ID NO: 333 - DNA, 14 bp, Homo sapiens
gcctcccaaagtgc

SEQ ID NO: 334 - DNA, 14 bp, Homo sapiens
aatcccagcacttt

SEQ ID NO: 335 - DNA, 14 bp, Homo sapiens
ctcccaaagtgctg

SEQ ID NO: 336 - DNA, 14 bp, Homo sapiens
cagcactttgggag

SEQ ID NO: 337 - DNA, 14 bp, Homo sapiens
caaagtgctgggat

SEQ ID NO: 338 - DNA, 14 bp, Homo sapiens
tgcactccagcctg

SEQ ID NO: 339 - DNA, 14 bp, Homo sapiens
atcccagcactttg

SEQ ID NO: 340 - DNA, 14 bp, Homo sapiens
tcccaaagtgctgg

SEQ ID NO: 341 - DNA, 14 bp, Homo sapiens
caggctggagtgca

SEQ ID NO: 342 - DNA, 14 bp, Homo sapiens
ctgcctcagcctcc

SEQ ID NO: 343 - DNA, 14 bp, Homo sapiens
ccagcactttggga

SEQ ID NO: 344 - DNA, 14 bp, Homo sapiens
ggaggctgaggcag

SEQ ID NO: 345 - DNA, 14 bp, Homo sapiens
tgctgggattacag

SEQ ID NO: 346 - DNA, 14 bp, Homo sapiens
ctgtaatcccagca

SEQ ID NO: 347 - DNA, 14 bp, Homo sapiens
ccaaagtgctggga

SEQ ID NO: 348 - DNA, 14 bp, Homo sapiens
gcactccagcctgg

SEQ ID NO: 349 - DNA, 14 bp, Homo sapiens
ccaggctggagtgc

SEQ ID NO: 350 - DNA, 14 bp, Homo sapiens
tcccagcactttgg

SEQ ID NO: 351 - DNA, 14 bp, Homo sapiens
gtgctgggattaca

SEQ ID NO: 352 - DNA, 14 bp, Homo sapiens
agtgctgggattac

SEQ ID NO: 353 - DNA, 14 bp, Homo sapiens
cctgcctcagcctc

SEQ ID NO: 354 - DNA, 14 bp, Homo sapiens
tgtaatcccagcac

SEQ ID NO: 355 - DNA, 14 bp, Homo sapiens
gtaatcccagcact

SEQ ID NO: 356 - DNA, 14 bp, Homo sapiens
gaggctgaggcagg

SEQ ID NO: 357 - DNA, 14 bp, Homo sapiens
cccaaagtgctggg

SEQ ID NO: 358 - DNA, 14 bp, Homo sapiens
cccagcactttggg

SEQ ID NO: 359 - DNA, 14 bp, Homo sapiens
atatatatatatat

SEQ ID NO: 360 - DNA, 14 bp, Homo sapiens
tatatatatatata

SEQ ID NO: 361 - DNA, 14 bp, Homo sapiens
cccaggctggagtg

SEQ ID NO: 362 - DNA, 14 bp, Homo sapiens
cactccagcctggg

SEQ ID NO: 363 - DNA, 14 bp, Homo sapiens
ttttgtatttttag

SEQ ID NO: 364 - DNA, 14 bp, Homo sapiens
ctaaaaatacaaaa

SEQ ID NO: 365 - DNA, 14 bp, Homo sapiens
tcctgcctcagcct

SEQ ID NO: 366 - DNA, 14 bp, Homo sapiens
tttgtatttttagt

SEQ ID NO: 367 - DNA, 14 bp, Homo sapiens
aggctgaggcagga

SEQ ID NO: 368 - DNA, 14 bp, Homo sapiens
actaaaaatacaaa

SEQ ID NO: 369 - DNA, 14 bp, Homo sapiens
ttgtatttttagta

SEQ ID NO: 370 - DNA, 14 bp, Homo sapiens
ctcctgcctcagcc

SEQ ID NO: 371 - DNA, 14 bp, Homo sapiens
tatttttagtagag

SEQ ID NO: 372 - DNA, 14 bp, Homo sapiens
tactaaaaatacaa

SEQ ID NO: 373 - DNA, 14 bp, Homo sapiens
atttttagtagaga

SEQ ID NO: 374 - DNA, 14 bp, Homo sapiens
ctctactaaaaata

SEQ ID NO: 375 - DNA, 14 bp, Homo sapiens
tctctactaaaaat

SEQ ID NO: 376 - DNA, 14 bp, Homo sapiens
ggctgaggcaggag

SEQ ID NO: 377 - DNA, 14 bp, Homo sapiens
tttttttttttttg

SEQ ID NO: 378 - DNA, 14 bp, Homo sapiens
caaaaaaaaaaaaa

SEQ ID NO: 379 - DNA, 14 bp, Homo sapiens
tgtatttttagtag

SEQ ID NO: 380 - DNA, 14 bp, Homo sapiens
gtatttttagtaga

SEQ ID NO: 381 - DNA, 14 bp, Homo sapiens
tgaggcaggagaat

SEQ ID NO: 382 - DNA, 14 bp, Homo sapiens
attctcctgcctca

SEQ ID NO: 383 - DNA, 14 bp, Homo sapiens
gctggagtgcagtg

SEQ ID NO: 384 - DNA, 14 bp, Homo sapiens
ttctcctgcctcag

SEQ ID NO: 385 - DNA, 14 bp, Homo sapiens
ctgaggcaggagaa

SEQ ID NO: 386 - DNA, 14 bp, Homo sapiens
ctactaaaaataca

SEQ ID NO: 387 - DNA, 14 bp, Homo sapiens
tctactaaaaatac

SEQ ID NO: 388 - DNA, 14 bp, Homo sapiens
ctggagtgcagtgg

SEQ ID NO: 389 - DNA, 14 bp, Homo sapiens
ctgcactccagcct

SEQ ID NO: 390 - DNA, 14 bp, Homo sapiens
cactgcactccagc

SEQ ID NO: 391 - DNA, 14 bp, Homo sapiens
aggctggagtgcag

SEQ ID NO: 392 - DNA, 14 bp, Homo sapiens
tttttgtattttta

SEQ ID NO: 393 - DNA, 14 bp, Homo sapiens
ccactgcactccag

SEQ ID NO: 394 - DNA, 14 bp, Homo sapiens
taaaaatacaaaaa

SEQ ID NO: 395 - DNA, 14 bp, Homo sapiens
ggctggagtgcagt

SEQ ID NO: 396 - DNA, 14 bp, Homo sapiens
actgcactccagcc

SEQ ID NO: 397 - DNA, 14 bp, Homo sapiens
tctcctgcctcagc

SEQ ID NO: 398 - DNA, 14 bp, Homo sapiens
gctgaggcaggaga

SEQ ID NO: 399 - DNA, 14 bp, Homo sapiens
aaaaaaaaaaaaag

SEQ ID NO: 400 - DNA, 14 bp, Homo sapiens
cttttttttttttt

SEQ ID NO: 401 - DNA, 14 bp, Homo sapiens
tgcctcagcctccc

SEQ ID NO: 402 - DNA, 14 bp, Homo sapiens
gggaggctgaggca

SEQ ID NO: 403 - DNA, 14 bp, Homo sapiens
cactttgggaggcc

SEQ ID NO: 404 - DNA, 14 bp, Homo sapiens
ggcctcccaaagtg

SEQ ID NO: 405 - DNA, 14 bp, Homo sapiens
cctcagcctcccaa

SEQ ID NO: 406 - DNA, 14 bp, Homo sapiens
ctcactgcaacctc

SEQ ID NO: 407 - DNA, 14 bp, Homo sapiens
gctcactgcaacct

SEQ ID NO: 408 - DNA, 14 bp, Homo sapiens
gaggttgcagtgag

SEQ ID NO: 409 - DNA, 14 bp, Homo sapiens
aggttgcagtgagc

SEQ ID NO: 410 - DNA, 14 bp, Homo sapiens
ttttttttttttga

SEQ ID NO: 411 - DNA, 14 bp, Homo sapiens
ttgggaggctgagg

SEQ ID NO: 412 - DNA, 14 bp, Homo sapiens
tcaaaaaaaaaaaa

SEQ ID NO: 413 - DNA, 14 bp, Homo sapiens
tttttttttttgag

SEQ ID NO: 414 - DNA, 14 bp, Homo sapiens
ttttttttttgaga

SEQ ID NO: 415 - DNA, 14 bp, Homo sapiens
ctcaaaaaaaaaaa

SEQ ID NO: 416 - DNA, 14 bp, Homo sapiens
tctcaaaaaaaaaa

SEQ ID NO: 417 - DNA, 290 bp, Homo sapiens; identical to SEQ ID NO: 280

SEQ ID NO: 418 - DNA, 312 bp, Homo sapiens
ggccgggcgcggtggctcacgcctgtaatcccagcactttgggaggccgaggcgggaggattgcttgagcccaggagttcgagaccagcctgggcaacatagcgagaccccgtctctacaaaaaatacaaaaattagccgggcgtggtggcgcgcgcctgtagtcccagctactcgggaggctgaggcaggaggatcgcttgagcccaggagttcgaggctgcagtgagctatgatcgcgccactgcactccagcctgggcgacagagcgagaccctgtctcaaaaaaaaaaaaaaaaaaaaaaaaaaaaaa

SEQ ID NO: 419 - DNA, 290 bp, Homo sapiens
  misc_feature (259)..(259): n is a, c, g, or t
gccgggcgcggtggctcacgcctgtaatcccagcactgtgggaggctgaggcgggaggattgctcgagctcaggagttcgaggctcgtctgagcgagagtgagaccccgactcatggaaaaaaatggaaaaacccagccgggcgccgcggcgagcgcctgtaatcccagcggcttcggaggctgaggcagcaggatgcccacaagccggagtctgaggttgcagtgagctacgacgccactgcactctgctcagggcanagggtagaactctgtctcgacaaaaaaaaaa

SEQ ID NO: 420 - DNA, 299 bp, Homo sapiens
agccgggctcggtggctcagcctgtaatcccagcactttgggaggctgaggtgagtggattgcctgagcccgcgggttcgagacccgcctgggcaacttggcgagacctcatctctacaataaatcaaaaaattagccgggcgtggtagcgcgcgcctgtagttccagctacttggaaggctgaggcggaaggatcgccggagcccagcaggtcgaggctgcggtggccgggagcggccactgcactccagtctgggcgacagagtgagactccaactcaaaaaaaaaaaaaaaaaaaa

SEQ ID NO: 421 - DNA, 237 bp, Homo sapiens
ggccgggcgcggtggctcacgcctgtaatcctagcactctgggaggccgaggcgggtggattgcttgagctcacgagttcgagaccagcctgagcaaaagcgagaccccgtctctactaaaaatagaaaaactgaggcaagaggatcgcttgagcccaagagttggaggttgctgtgagctatgacgccacggcactctacccagggcgacagcttgagactctgtctcaaaaaaaa

SEQ ID NO: 422 - DNA, 298 bp, Homo sapiens
ggccgggcgcggtggctcacgcctgtaatcccagcactttgggaggccgaggcgggaggattgcttgagcccaggagttcgagaccagcctgggcaacatagcgagacctcgtctctacaaaaaattaaaaaattagccgggcgtggtggcgcgcgcctgtagtcccagctactcgggaggctgaggcgggaggatcgcctgagcccaggaggtcgaggctgcggtgagccgtgatcgtgccactgcactccagcctgggcgacagagtgagaccccgactcaaaaaaaaaaaaaaaa

SEQ ID NO: 423 - DNA, 298 bp, Homo sapiens
  misc_feature (262)..(262): n is a, c, g, or t
ggccgggcgcagtggctcagcctgtaatcccagcactttgggaggccgaggcgagaggattgcctgagcccgggggttcgagaccagcctgggcaacttggtgagaccttgtctctacaataaataaaaaattagccaggcgtggtagcgcgcgcctgtagttccagctacttggaaggctgaggcggaaggatcgcctgggcccagcaggttggggctgcggtggccgtgagcatgccactgcactacggcatggggggangagactccaaatcttaaaaaagtctgaaaaggaaga

SEQ ID NO: 424 - DNA, 283 bp, Homo sapiens
ggccgggcgcggtggctcacgcctgtaatcccagcactttgggaggccgaggcgggaggatcacttgagcccaggagttcgagaccagcctgggcaacatggtgaaaccccgtctctacaaaaaatacaaaaattagccgggcgtggtggcgcgcgcctgtagtcccagctactcgggaggctgaggcaggaggatcgcttgagcccgggaggtcgaggctgcagtgagccgtgatcgcgccactgcactccagcctgggcgacagagcgagaccctgtctca

SEQ ID NO: 425 - DNA, 283 bp, Homo sapiens
ggccgggcgcggtggctcacgcctgtaatcccagcactttgggaggccgaggcgggaggattgcttgagcccaggagttcgagaccagcctgggcaacatagcgagaccccgtctctacaaaaaatacaaaaattagccgggcgtggtggcgcgcgcctgtagtcccagctactcgggaggctgaggcaggaggatcgcttgagcccaggagttcgaggctgcagtgagctatgatcgcgccactgcactccagcctgggcgacagagcgagaccctgtctca

SEQ ID NO: 426 - DNA, 312 bp, Homo sapiens
ggccgggcgcggtggctcacgcctgtaatcccagcactttgggaggccgaggcgggaggatcgcttgaggccaggagttcgagaccagcctgggcaacatagcgagaccccgtctctacaaaaaatataaaaattagccgggcgtggtggcgcgcgcctgtagtcccagctactcgggaggctgaggcgggaggatcgcttgagcccaggagttcgaggctgcagtgagctatgatcgcgccactgcactccagcctgggcgacagagcgagaccctgtctcaaaaaaaaaaaaaaaaaaaaaaaaaaaaaa

SEQ ID NO: 427 - DNA, 312 bp, Homo sapiens
  misc_feature (127)..(127): n is a, c, g, or t
ggccgggcgcggtggctcacgcctgtaatcccagcactttgggaggccgaggcgggaggatcgcttgaggccaggagttcgagaccagcctgggcaacatagcgagaccccgtctctacaaaaaatntaaaaattagccgggcgtggtggcgcgcgcctgtagtcctagctactcgggaggctgaggcaggaggatcgcttgagcccaggagttcgaggttacagtgagctatgatcgcgccactgcactccagcctgggcgacagagcgagaccctgtctcaaaaaaaaaaaaaaaaaaaaaaaaaaaaaa

SEQ ID NO: 428 - DNA, 329 bp, Homo sapiens
ggccgggcgcggtggctcacgcctgtaatcctagcactctgggaggccgaggcgggaggatcgctcgaggtcaggagttcgaaaccagcctgagcaagagcgagaccccgtctctactaaaaatagaaagaaattaattggccaactaaaaatatatagaaaaaattagccgggcatggtggcgcatgcctgtagtcccagctactcgggaggctgaggcaggaggatcgcttgagcccaggagtttgaggttgctgtgagctaggctgacgccacggcactctagcccgggcaacagagtgagactctgtctcaaaaaaaaaaaaaaa

SEQ ID NO: 429 - DNA, 310 bp, Homo sapiens
ggccgggcgcggtggctcaagcctgtaatcccagcactttgggaggccgagacgggcggatcacgaggtcaggagatcgagaccatcctggctaacacggtgaaaccccgtctctactaaaaaatacaaaaaactagccgggcgaggtggcgggcgcctgtagtcccagctactcgggaggctgaggcaggagaatggcgtgaacccgggaggcggagcttgcagtgagctgagatccggccactgcactccagcctgggcgacagagcgagactccgtctcaaaaaaaaaaaaaaaaaaaaaaaaaaaa

SEQ ID NO: 430 - DNA, 297 bp, Homo sapiens
ggccgggcgcggtggctcacgcctgtaatcccagcactttgggaggccgaggcgggcggatcacaaggtcaggagatcgagaccacggtgaaaccccgtctctactaaaaatacaaaaaattagccgggcgcggttgtgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaaaaaaa

SEQ ID NO: 431 - DNA, 297 bp, Homo sapiens
ggccgggcgcggtggctcaagcctgtaatcccagcactttgggaggccgaggcgggcggatcacaaggtcaggagatcgagaccacagtgaaaccccgtctctactaaaaatacaaaaaattagccgggcgcggtggcgggcgcctgtagtcccagctactcaggaggctgaggcaggagaatggcgggaacccgggaggcggagcttgcagtgagccgagatcgcgccactgcactccagcctgggcaacagcgtgagactccgtctcaaaaaaaaaaaaaaaaaaaaaaaaaaaa

SEQ ID NO: 432 - DNA, 280 bp, Homo sapiens
ggccgggcgcggtggctcacgcctgtaatcccagcactttgggaggccgaggcgggcggatcacgaggtcaagagatcgagaccatcctggccaacatggtgaaaccccgtctctactaaaaatacaaaaattagctgggcgtggtggcgcgcgcctgtagtcccagctactcgggaggctgaggcaggagaatcgcttgaacccgggaggcggaggttgcagtgagccgagatcgcgccactgcactccagcctggcgacagagcgagactccgtctca

SEQ ID NO: 433 - DNA, 312 bp, Homo sapiens
ggccgggcgcggtggctcacgcctgtaatcccagcactttgggaggccgaggcgggcggatcacgaggtcaggagatcgagaccatcctggccaacatggtgaaaccccgtctctactaaaaatacaaaaattagctgggcgtggtggcgcgtgcctgtaatcccagctactcgggaggctgaggcaggagaatcgcttgaaccagggagtcggaggttgcagtgagccgagatcgcgccactgcactccagcctggcgacagagcgagactccgtctcaaaaaaaaaaaaaaaaaaaaaaaaaaaaaaaaa

SEQ ID NO: 434 - DNA, 312 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgtggtggcacgcgcctgtagtcccagctactcgggaggctgaggcaggagaatcgcttgaacccgggaggcggaggttgcagtgagccgagatcgcgccactgcactccagcctgggcgacagagcgagactccgtctcaaaaaaaaaaaaaaaaaaaaaaaaaaaaaaa

SEQ ID NO: 435 - DNA, 281 bp, Homo sapiens
ggccgggcgcggtggctcacgcctgtaatcccagcactttgggaggccgaggcgggcggatcacgaggtcaggagttcgagaccagcctggccaacatggtgaaaccccgtctctactaaaaatacaaaaattagccgggcgtggtggcgcgcgcctgtaatcccagctactcgggaggctgaggcaggagaatcgcttgaacccgggaggcggaggttgcagtgagccgagatcgcgccactgcactccagcctgggcgacagagcgagactccgtctca

SEQ ID NO: 436 - DNA, 280 bp, Homo sapiens
rgccgggcgcggtggctcacgcctgtaatcccagcactttgggaggccgaggcgggcggatcacgaggtcaggagttcgagaccagcctggccaagatggtgaaaccccgtctctactaaaaatacaaaaattagccgggcgtggtggcgcgggcctgtaatcccagctactcgggaggctgaggcagagaatcgcttgaacccgggaggcggaggttgcagtgagccgagatcgcgccactgcactccagcctgggcgacagagcgagactccgtctca

SEQ ID NO: 437 - DNA, 312 bp, Homo sapiens
ggccgggcgcggtggctcacgcctgtaatcccagcactttgggaggccgaggcgggtggatcacgaggtcaggagttcgagaccagcctggccaagatggtgaaaccccgtctctactaaaaatacaaaaattagccgggcgcggtggcgggcgcctgtaatcccagctactcgggaggctgaggcaggagaatcgcttgaacccgggaggcggaggttgcagtgagccgagatcgcgccactgcactccagcctgggcgacagagcgagactccgtctcaaaaaaaaaaaaaaaaaaaaaaaaaaaaaaaa

SEQ ID NO: 438 - DNA, 312 bp, Homo sapiens
ggccgggcgcggtggctcacgcctgtaatcccagcactttgggaggccgaggcgggtggatcacgaggtcaggagttcaagaccagcctggccaagatggtgaaaccccgtctctactaaaaatacaaaaattagccgggcgtggtggcgggcgcctgtaatcccagctactcgggaggctgaggcagagaattgcttgaacccgggaggcggaggttgcagtgagccgagatcgcgccactgcactccagcctgggcgacagagcgagactccgtctcaaaaaaaaaaaaaaaaaaaaaaaaaaaaaaaaa

SEQ ID NO: 439 - DNA, 284 bp, Homo sapiens
ggccgggcgcggtggctcacgcctgtaatcccagcactttgggaggccgaggcgggcggatcacctgaggtcgggagttcgagaccagcctgaccaacatggagaaaccccgtctctactaaaaatacaaaaattagccgggcgtggtggcgcatgcctgtaatcccagctactcgggaggctgaggcaggagaatcgcttgaacccgggaggcggaggttgcggtgagccgagatcgcgccattgcactccagcctgggcaacaagagcgaaactccgtctca

SEQ ID NO: 440 - DNA, 284 bp, Homo sapiens
ggccgggcgcggtggctcacgcctgtaatcccagcactttgggaggccgaggcgggtggatcacctgaggtcaggagttcgagaccagcctggccaacatggtgaaaccccgtctctactaaaaatacaaaaattagccgggcgtggtggcgggcgcctgtaatcccagctactcgggaggctgaggcaggagaatcgcttgaacccgggaggcggaggttgcagtgagccgagatcgcgccactgcactccagcctgggcaacaagagcgaaactccgtctca

SEQ ID NO: 441 - DNA, 312 bp, Homo sapiens
ggccgggcgcggtggctcacgcctgtaatcccagcactttgggaggccgaggcgggtggatcacctgaggtcaggagttctagaccagcctggccaacatggtgaaaccccgtctctactaaaaatacaaaaattagccgggcgtggtggcaggcgcctgtaatcccagctactcggggggccgaggcaggagaatcgcttgaacccgggaggcggaggttgcagtgagccgagatcgcgccatcgcactccagcctgggggacaagagcgagacttcgtctcaaaaaaaaaaaaaaaaaaaaaaaaaaaaa

SEQ ID NO: 442 - DNA, 312 bp, Homo sapiens
ggccgggcgcggtggctcacgcctgtaatcccagcactttgggaggccgaggcgggcggatcacctgaggtcaggagttcgagaccagcctggccaacatggtgaaaccccgtctctactaaaaatacaaaaattagccgggcgtggtggcgggcgcctgtaatcccagctactcgggaggctgaggcaggagaatcgcttgaacccgggaggcggaggttgcagtgagccgagatcgcgccattgcactccagcctgggcgacaagagcgaaactccgtctcaaaaaaaaaaaaaaaaaaaaaaaaaaaaa

SEQ ID NO: 443 - DNA, 312 bp, Homo sapiens
ggccgggcgcggtggctcacgcctgtaatcccagcactttgggaggccgaggcgggcggatcactgaggtcaggagttcgagaccagcctggccaacatggtgaaaccccgtctctactaaaaatacaaaaattagccgggcgtggtggcgggcgcctgtaatcccagctactcgggaggctgaggcaggagaatcgcttgaaaccggaaggcggaggttgcagtgagccgagatcgcgccactgcactccagcctgggcaacaagagcgaaactccgtctcaaaaaaaaaaaaaaaaaaaaaaaaaaaaaa

SEQ ID NO: 444 - DNA, 283 bp, Homo sapiens
ggccgggcgcggtggctcacgcctgtaatcccagcactttgggaggccgaggcgggcggatcacctgaggtcaggagttcgagaccagcctggccaacatggtgaaaccccgtctctactaaaaatacaaaaattagccgggcgtggtggcgcgcgcctgtaatcccagctactcgggaggctgaggcaggagaatcgcttgaacccgggaggcggaggttgcagtgagccgagatcgcgccactgcactccagcctgggcgacagagcgagactccgtctca

SEQ ID NO: 445 - DNA, 312 bp, Homo sapiens
ggccgggcgcggtggctcacgcctgtaatcccagcactttgggaggccgaggcgggcggatcacctgaggtcaggagttcgagaccagcctggccaacatggtgaaaccccgtctctactaaaaatacaaaaattagccgggcgtggtggcgggcgcctgtaatcccagctactcgggaggctgaggcaggagaatcgcttgaacccgggaggcggaggttgcagtgagccgagatcgcgccactgcactccagcctgggcgacagagcgagactccgtctcaaaaaaaaaaaaaaaaaaaaaaaaaaaaaa

SEQ ID NO: 446 - DNA, 312 bp, Homo sapiens
ggccgggcgcggtggctcacgcctgtaatcccagcactttgggaggccgaggcgggcggatcacgaggtcaggagttcgagaccagcctggccaacatggtgaaaccccgtctctactaaaaatacaaaaaattagccgggcgtggtggcgcgcgcctgtagtcccagctactcgggaggctgaggcaggagaatcgcttgaacccgggaggcggaggttgcagtgagccgagatcgcgccactgcactccagcctgggcgacagagcgagactccgtctcaaaaaaaaaaaaaaaaaaaaaaaaaaaaaaa

SEQ ID NO: 447 - DNA, 312 bp, Homo sapiens
ggccgggcgcggtggctcacgcctgtaatcccagcactttgggaggccgaggcgggcggatcactgaggtcaggagttcgagaccagcctggccaatatggtgaaaccccgtctctactaaaaatacaaaaattagccgggcgtggtggcgcgcgcctgtagtcccagctactcgggaggctgaggcagaagaatcgcttgaacccgggaggcggaggttgcagtgagccgagatcgcgccactgcactccagcctgggcgacagagcgagactccgtctcaaaaaaaaaaaaaaaaaaaaaaaaaaaaaaa

SEQ ID NO: 448 - DNA, 283 bp, Homo sapiens
ggccgggcgcggtggctcacgcctgtaatcccagcactttgggaggccgaggcgggcggatcacttgaggtcaggagttcgagaccagcctggccaacatggtgaaaccccgtctctactaaaaatacaaaaattagccgggcgtggtggcgcgcgcctgtaatcccagctactcgggaggctgaggcaggagaatcgcttgaacccgggaggcggaggttgcagtgagccgagatcgcgccactgcactccagcctgggcgacagagcgagactccgtctca

SEQ ID NO: 449 - DNA, 312 bp, Homo sapiens
ggccgggcgcggtggctcacgcctgtaatcccagcactttgggaggccgaggcgggcggatcacttgaggccaggagttcgagaccagcctggccaacatggcaaaaccccgtctctactaaaaatacaaaaattagccgggcgtggtggcgcgcgcctgtaatcccagctactcgggaggctgaggcacgagaatcgcttgaacccgggaggcggaggttgcagtgagccgagatcgcgccactgcactccagcctgggcgacagagcgagactctgtctcaaaaaaaaaaaaaaaaaaaaaaaaaaaaaa

SEQ ID NO: 450 - DNA, 282 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgtggtggcgggcgcctgtagtcccagctactcgggaggctgaggcaggagaatggcgtgaacccgggaggcggagcttgcagtgagccgagatcgcgccactgcactccagcctgggcgacagagcgagactccgtctca

SEQ ID NO: 451 - DNA, 282 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgtggtggcgggcgcctgtagtcccagctactcgggaggctgaggcaggagaatggcgtgaacccgggaggcggagcttgcagtgagccgagatcccgccactgcactccagcctgggcgacagagcgagactccgtctca

SEQ ID NO: 452 - DNA, 282 bp, Homo sapiens
ggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcgcgccactgcactccagcctgggcgacagagcgagactccgtctca

SEQ ID NO: 453 - DNA, 282 bp, Homo sapiens
ggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagactccgtctca

SEQ ID NO: 454 - DNA, 281 bp, Homo sapiens
ggccgggcgcggtggctcacgcctgtaatcccagcactttgggaggccgaggcgggcggatcacgaggtcaggagatcgagaccatcccggctaaaacggtgaaaccccgtctctactaaaactacaaaaaatagccgggcgtagtggcgggcgcctgtagtcctagctacttgggaggctgaggcaggagaatggcgtgaacccgggaggcggagcttgcagtgagccgagatcccgccactgcactccagcctgggcgacagagcgagactccgtctca

SEQ ID NO: 455 - DNA, 282 bp, Homo sapiens
ggccgggcgcggtggctcacgcctgtaatcccagcactttgggaggccgaggcgggtggatcacgaggtcaggagatcgagaccatcctggctaacacggtgaaaccccgtctctactaaaaatacaaaaaattagccgggcgtggtggcgggcgcctgtagtcccagctactcgggaggctgaggcaggagaatggcgtgaacccgggaggcggagcttgcagtgagccgagattgcgccactgcagtccagcctgggcgacagagcgagactccgtctca

SEQ ID NO: 456 - DNA, 282 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgcggtggcgggcgcctgtagtcccagctactcgggaggctgaggcaggagaatggcgtgaacccgggaggcggagcttgcagtgagccgagatagcgccactgcagtccggcctgggcgaaagagcgagactccgtctca

SEQ ID NO: 457 - DNA, 289 bp, Homo sapiens
ggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccgggaagcggagcttgcagtgagccgagattgcgccactgcagtccgcagtccggcctgggcgacagagcgagactccgtctca

SEQ ID NO: 458 - DNA, 289 bp, Homo sapiens
ggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggcgggcgcctgtagtcccagctactggggaggctgaggcaggagaatggcgtgaacccgggaagcggagcttgcagtgagccgagattgcgccactgcagtccgcagtccggcctgggcgacagagcgagactccgtctca

SEQ ID NO: 459 - DNA, 283 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaaattagccgggcgtggtagcgggcgcctgtagtcccagctactcgggaggctgaggcaggagaatggcgtgaacccgggaggcggagcttgcagtgagccaagatagcgccactgcagtccagcctgggcgaaagagcgagactccgtctca

SEQ ID NO: 460 - DNA, 282 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctca

SEQ ID NO: 461 - DNA, 282 bp, Homo sapiens
ggccgggcgcggtggctcacgcctgtaatcccagcactttgggaggccgaggcgggcggatcacgaggtcaggagatcgagaccatcctggctaacaaggtgaaaccccgtctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctca

SEQ ID NO: 462 - DNA, 299 bp, Homo sapiens
rgccgggcgcggtggctcacgcctgtaatcccagcactttgggaggccgaggcgggcggatcacgaggtcaggagatcgagaccacggtgaaaccccgtctctactaaaaatacaaaaaattagccgggcgcagtggcgggcgcctgtagtcccagctactcgggaggctgaggcaggagaatggcgtgaacccggaaggcggagcttgcagtgagcggagatcgcgccacagcactcccgcctgggcgacagagcgagactccgtctcaaaaaaaaaaaaaaaaaaaaaaaaaaaaaa

SEQ ID NO: 463 - DNA, 270 bp, Homo sapiens
ggccgggcgcggtggctcacgcctgtaatcccagcactttgggaggccgaggcgggcggatcacgaggtcaggagatcgagaccacggtgaaaccccgtctctactaaaaatacaaaaaattagccgggcgcggtggcgggcgcctgtagtcccagctactcgggaggctgaggcaggagaatggcgtgaacccgggaggcggagcttgcagtgagccgagatcgcgccactgcactccagcctgggcgacagagcgagactccgtctca

SEQ ID NO: 464 - DNA, 270 bp, Homo sapiens
ggccgggcgcggtggctcacgcttgtaatcccagcactttgggaggccgaggcgggcggatcacgaggtcaggagatcgagaccacggtgaaaccccgtctctactaaaaatacaaaaaattagccgggcgcggtggcgggcgcctgtagtcccagctactcgggaggctgaggcaggagaatggcgtgaacccgggaggcggagcttgcagtgagccgagatcgcgccactgcactccagcctgggcgacagagcgagactccgtctca

SEQ ID NO: 465 - DNA, 271 bp, Homo sapiens
ggccgggcgcggtggctcacgcttgtaatcccagcactttgggaggccgaggcgggcggatcacgaggtcaggagatcgagaccacggtgaaaccccgtctctactaaaaatacaaaaaattagccgggcgcggtggcgggcgcctgtagtcccagctactcggagaggctgaggcaggagaatggcgtgaacccgggaggcggagcttgcagtgagccgagatcgcgccactgcactccagcctgggcgacagagcgagactccgtctca

SEQ ID NO: 466 - DNA, 272 bp, Homo sapiens
ggccgggcgcggtggctcacgcttgtaatcccagcactttgggaggccgaggcgggcggatcacgaggtcaggagatcgagaccacggtgaaaccccgtctctactaaaaatacaaaaaaattagccgggcgtggtggcgggcgcctgtagtcccagctactcggagaggctgaggcaggagaatggcgtgaacccgggaggcggagcttgcagtgagccgagattgcgccactgcactccagcctgggcgacagagcgagactccgtctca

SEQ ID NO: 467 - DNA, 270 bp, Homo sapiens
ggccgggcgcggtggctcacgcctgtaatcccagcactttgggaggccgaggcgggcggatcacgaggtcaggagatcgagaccacggtgaaaccccgtctctactaaaaatacaaaaaattagccgggcgcagtggcgggcgcctgtagtcccagctactcgggaggctgaggcaggagaatggcgtgaacccggaaggcggagcttgcagtgagcggagatcgcgccacagcactcccgcctgggcgacagaacgagactccgtctca

SEQ ID NO: 468 - DNA, 280 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgaggtggcgggcgcctgtagtcccagctactcgggaggctgaggcaggagaatggcgtgaacccgggaggcggagcttgcagtgagccgagatcgcgccactgcactccagcctgggcgacagcgagactccgtctca

SEQ ID NO: 469 - DNA, 281 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgaggtggcgggcgcctgtagtcccagctactcgggaggctgaggcaggagaatggcgtgaaccccggggggcggagcctgcagtgagccgagatcgcgccactgcactccagcctgggcgacagcgagactccgtctca

SEQ ID NO: 470 - DNA, 282 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgtggtggcgggcgcctgtagtcccagctacgcgggaggctgaggcaggagaatggcgtgaacccgggaggcggagcttgcagtgagccgagatcgcgccactgcactccagcctgggcgacagagcgagactccgtctca

SEQ ID NO: 471 - DNA, 284 bp, Homo sapiens
ggccgggcgcggtggctcacgcctgtaatcccagcactttgggaggccgaggcgggcggatcacgaggtcaggagatcgagaccatcctggctaacacagtgaaaccccgtctctactaaaaaacacaaaaaaattagccgggcgtggtggcgggcgcctgtagtcccagctacgcgggaggctgaggcaggagaatggcgtgaacccgggaggcggagcttgcagtgagccgagatcgcgccactgcactccagcctgggcgacagagcgagactccgtctca

SEQ ID NO: 472 - DNA, 312 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgaggtggcgggcgcctgtagtcccagctacgcgggaggctgaggcaggagaatggcgtgaaccccggggggcggagcctgcagtgagccgagatcgcgccactgcactccagcctgggcgacagcgagactccgtctcaaaaaaaaaaaaaaaaaaaaaaaaaaaaaaaa

SEQ ID NO: 473 - DNA, 282 bp, Homo sapiens
ggccgggcgcggtggctcacgcctgtaatcccagcactttgggaggccgagacgggcggatcacgaggtcaggagatcgagaccatcctggctaacacggtgaaaccccgtctctactaaaaatacaaaaaattagccgggcatggtggcgcgcgcctgtagtcccagctacacgggaggctgaggcaggagaatggcgtgaacccgggaggcggagcttgcagtgagtcgagatcgcgccactgcactccagcctgggcgacagagcgaaactccgtctca

SEQ ID NO: 474 - DNA, 282 bp, Homo sapiens
ggccgggcgcggtggctcacgcctgtaatcccagcactttgggaggcagaggcgggcggatcatgaggtcaggagatcgagaccatcctggctaacgcggtgaaaccccgcctctactaaaaatacaaaaaattagccgggcgtggtggcgggcgcctgtggtcccggctactcgggaggctgaggcaggagaatggcgtgaacccgggaggcggagcttgcagtgagctgaggtcgcgccactgcaccccagcctgggcgacagagcgagactccgtctca

SEQ ID NO: 475 - DNA, 283 bp, Homo sapiens
ggccgggcgcggtggctcacgcttgtaatcccagcactttgggaggccgaggcgggcggatcacgaggtcaggagatcgagaccatcctggctaacacggtgaaaccccgtctctactaaaaatacaaaaaaattagccgggcgtgatggcgggcgcctgtagtcccagctactcgggaggctgaggcaggagaatggcgtgaacccgggaggcggagcttgcagtgagccgagattgcgccactgcactcccgcctgggccacagagcgagactccgtctca

SEQ ID NO: 476 - DNA, 312 bp, Homo sapiens
ggctgggcgcggtggctcacgcctgtaatcccagcactttgggaggccgaggcgggcggatcacaaggtcaggagatcgagaccatcttggctaacacggtgaaaccccgtctctactaaaaatacaaaaaattagccgggcgcggtggcgggcgcctgtagtcccagctactcgggaggctgaggcaggagaatggcgtgaacctgggaggcggagcttgcagtgagccgagattgcgccactgcaatccggcctgggctaaagagcgggactccgtctcaaaaaaaaaaaaaaaaaaaaaaaaaaaaaaa

SEQ ID NO: 477 - DNA, 312 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgtggtggtgggcgcctgtaatcccagctactcgggaggctgaggcaggagaatggcatgaacccaagaggcggagcttgcagtgagccgggatagcgccactgcagtccagcttgggcgaaagagtgagactccgtctcaaaaaaaaaaaaaaaaaaaaaaaaaaaaaaa

SEQ ID NO: 478 - DNA, 292 bp, Homo sapiens
ggccgggcgtggtggctcacgcctgtaatcccagcactttgggaggccgaggcgggtggatcatgaggtcaggagatcgagaccatcctggctaacacagtgaaaccccgtctctactaaaaatacaaaaaattagccgggagcggtggcgggctcctgtagtcccagctacttgagaggctgaggcaggagaatggcgtgaacccaggaggcggagcttgcagtgagccgagatcgcgccactgcactccagcctgggcgacagagccagacgctgtctcaaaaaaaaaaa

SEQ ID NO: 479 - DNA, 20 bp, Mus musculus
ctttaatcccagcacttggg

SEQ ID NO: 480 - DNA, 23 bp, Mus musculus
ctgtgtagccctggctgtcctgg

SEQ ID NO: 481 - DNA, 19 bp, Mus musculus
ggctgtcctggaactcact

SEQ ID NO: 482 - DNA, 113 bp, Mus musculus
gcctttaatcccagcacttgggaggcagaggcaggcggatttctgagttcgaggccagcctggtctacaaagtgagttccaggacagccagggctacacagagaaaccctgtc

SEQ ID NO: 483 - DNA, 209 bp, Homo sapiens
aaatggtgctgggaaaactggctagccatatgtagaaagctgaaactggatcccttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggc

SEQ ID NO: 484 - DNA, 20 bp, Artificial Sequence
  Synthetic Construct
gccatcccatatattggaac

SEQ ID NO: 485 - DNA, 21 bp, Artificial Sequence
  Synthetic Construct
ctactgttcgcagtcatagcc

SEQ ID NO: 486 - DNA, 22 bp, Artificial Sequence
  Synthetic Construct
aatcgttcacctcctcttcctc

SEQ ID NO: 487 - DNA, 20 bp, Artificial Sequence
  Synthetic Construct
aaatggtgctgggaaaactg

SEQ ID NO: 488 - DNA, 20 bp, Artificial Sequence
  Synthetic Construct
gccattgcttttggtgtttt

SEQ ID NO: 489 - DNA, 20 bp, Artificial Sequence
  Synthetic Construct
aaatggtgctgggaaaactg

SEQ ID NO: 490 - DNA, 20 bp, Artificial Sequence
  Synthetic Construct
gccattgcttttggtgtttt

SEQ ID NO: 491 - DNA, 20 bp, Artificial Sequence
  Synthetic Construct
tggagtacagtggctcatgc

SEQ ID NO: 492 - DNA, 20 bp, Artificial Sequence
  Synthetic Construct
ccatcatgcctggctaattt

SEQ ID NO: 493 - DNA, 19 bp, Artificial Sequence
  Synthetic Construct
gagtgcaatggtgcgatct

SEQ ID NO: 494 - DNA, 20 bp, Artificial Sequence
  Synthetic Construct
gcagcatggtgaaaccctat

SEQ ID NO: 495 - DNA, 20 bp, Artificial Sequence
  Synthetic Construct
tgacgagttaatgggtgcag

SEQ ID NO: 496 - DNA, 21 bp, Artificial Sequence
  Synthetic Construct
agatccctggacacattctga

SEQ ID NO: 497 - DNA, 25 bp, Artificial Sequence
  Synthetic Construct
agcaatgatacaatgtgctatctga

SEQ ID NO: 498 - DNA, 27 bp, Artificial Sequence
  Synthetic Construct
tgttaattatcttatccttgtgccatt

SEQ ID NO: 499 - DNA, 20 bp, Artificial Sequence
  Synthetic Construct
acaccaccatatccctacgc

SEQ ID NO: 500 - DNA, 20 bp, Artificial Sequence
  Synthetic Construct
gggatcatggccttctaggt

SEQ ID NO: 501 - DNA, 150 bp, Homo sapiens
agcaatgatacaatgtgctatctgaaaaaaactaaactttttaaaatataaatttgaggggaaaatgaactaagttagtaatgttttgggaataagatcatgggttggccacaccaaccaggtaatggcacaaggataagataattaaca